(19) United States
(12) Patent Application Publication
(10) Pub. No.: US 2025/0229001 A1
(43) Pub. Date: Jul. 17, 2025

(54) ANCHORED CELL CONSTRUCT CULTURE

(71) Applicant: CAROMEATS INC., Kitchener (CA)

(72) Inventor: Alireza SHAHIN-SHAMSABADI, Kitchener (CA)

(21) Appl. No.: 19/057,909

(22) Filed: Feb. 19, 2025

Related U.S. Application Data
(63) Continuation-in-part of application No. 18/677,517, filed on May 29, 2024.
(60) Provisional application No. 63/707,874, filed on Oct. 16, 2024.
(60) Provisional application No. 63/733,255, filed on Dec. 12, 2024.
(60) Provisional application No. 63/504,774, filed on May 29, 2023.
(60) Provisional application No. 63/504,827, filed on May 30, 2023.
(60) Provisional application No. 63/506,240, filed on Jun. 5, 2023.
(60) Provisional application No. 63/510,949, filed on Jun. 29, 2023.
(60) Provisional application No. 63/517,188, filed on Aug. 2, 2023.
(60) Provisional application No. 63/583,488, filed on Sep. 18, 2023.
(60) Provisional application No. 63/591,004, filed on Oct. 17, 2023.

(51) Int. Cl. A61L 27/38; A61L 27/54; C12N 5/00; C12N 5/077

(52) U.S. Cl. CPC A61L 27/3826; A61L 27/54; C12N 5/0062; C12N 5/0652

(57) ABSTRACT

Scaffold-free cell constructs are made from cell sheets. A device has a membrane and anchors located inside of a perimeter or wall of the membrane. The membrane allows cell attachment followed by detachment of cells and secreted ECM as a cell sheet. In a method, cells are grown into a cell sheet. The cell sheet is located around two or more anchors. The cell sheet remodels into a cell construct of a different shape attached to the anchors. When a cell sheet is grown in the device, the anchors form and pass through openings in the cell sheet. Detaching the cell sheet locates the cell sheet around the around the anchors. The cell construct can be used with or without decellularization and while attached to the anchors, for example as in vitro models, or after being removed from the anchors, for example in regenerative medicine or tissue engineering.

### RELATED APPLICATIONS

This application is a continuation-in-part of U.S. application Ser. No. 18/677,517 filed on May 29, 2024, which claims the benefit of U.S. provisional application 63/504,774 filed on May 29, 2023; U.S. provisional application 63/504,827 filed on May 30, 2023; U.S. provisional application 63/506,240 filed on Jun. 5, 2023; U.S. provisional application 63/510,949 filed on Jun. 29, 2023; U.S. provisional application 63/517,188 filed on Aug. 2, 2023; U.S. provisional application 63/583,488 filed on Sep. 18, 2023; and, U.S. provisional application 63/591,004 filed on Oct. 17, 2023. This application also claims the benefit of U.S. provisional application 63/707,874 filed on Oct. 16, 2024 and U.S. provisional application 63/733,255 filed on Dec. 12, 2024. All of the applications mentioned in this paragraph are incorporated herein by reference.

### FIELD

This specification relates to cell culture devices and to methods of growing cell constructs, for example cell sheets or 3D cell constructs; to de-cellularized cell constructs; to cell constructs or de-cellularized cell constructs loaded with one or more therapeutic agents, and to methods of using the cell constructs, de-cellularized cell constructs or loaded cell constructs, for example by way of implanting in a patient or for drug screening; and to methods of in vivo drug, cell, gene or tissue therapy.

### BACKGROUND

US Publication 20040009566 describes a method of growing a cell sheet using a temperature responsive surface coated with poly(N-isopropylacrylamide) (PIPAAm). At 37° C., the surface is suitable for cell attachment. When the temperature is lowered to 20° C., the surface becomes hydrophilic and releases cell layers from the surface as a sheet.

International Publication Number WO 2022/000086 A1, Self-Assembled Cell Sheet Constructs and Methods of Making Thereof, describes a method of making a cell sheet. The method includes plating a plurality of cells on a substantially flat surface. The plurality of cells are grown starting with at least 80% confluency to form cell layers with preserved intercellular linkages and secreted extracellular matrix (ECM). A series of cell culture media having different pH are applied to obtain a substantially planar untethered cell sheet.

### Introduction

Cultured scaffold-free cell sheets have the ability to adhere to each other or other tissues and organs if implanted inside the body. However, cell sheets detached from a substrate and then cultured without an anchor tend to fold and form crumpled sheets or agglomerates over time in an uncontrolled way that is not reproducible. The transformation is not reversible and the agglomerates cannot be restored to their original flat, sheet-like shape. Cells in the agglomerates may have reduced viability and functionality, and may not accurately represent in vivo cells of the same type. However, cell layers that remain attached to a planar substrate in a bioreactor also may not accurately represent in vivo cells of the same type, for example due to unnatural basal-apical morphology of the cells while adhering to the substrate. The tendency of detached cell sheets to form agglomerates limits the potential applications of cell sheets for various uses, such as in vitro modeling and drug discovery. Detached cell sheets cultured without anchors are also fragile and hard to manipulate, for example moving the sheets for an in vivo application is difficult.

This specification describes cell culture devices and methods for making a cell construct. The cell constructs are made from cell sheets and are optionally scaffold-free. The cell constructs may be 3D cell constructs such as fibers or sheets that are thicker or of a different shape than the precursor cell sheet. A device for growing a cell construct may have a membrane and a plurality of anchors. The anchors are located inside of a perimeter of the membrane, which is optionally defined by a wall. The membrane may be treated or formed to first allow cell attachment and then to facilitate detachment of cells and their secreted ECM as a cell sheet from the membrane. In a method of growing a cell construct, cells are grown, optionally in layers, and attach or fuse to each other to form a cell sheet. The detached cell sheet is located around two or more anchors. With continued cell culture (e.g. growth, differentiation and/or remodeling), the cell sheet forms a cell construct of a different shape attached to the anchors. When a cell sheet is grown in the device, the anchors may form and pass through openings in the cell sheet. Detaching the cell sheet from the membrane locates the cell sheet around the anchors, which then allows the cell sheet to form a remodeled construct attached to the anchors. The anchored constructs can be used with or without decellularization and while attached to the anchors, for example as in vitro models, or after being removed from the anchors, for example in in vivo applications such as regenerative medicine or tissue engineering.

In some embodiments, a device for growing cells includes a membrane (i.e. a solid porous or non-porous substrate) with a region suitable for growing a cell sheet. The membrane has a plurality of anchors or is associated with a plurality of anchors. In some embodiments, the anchors are protuberances, optionally in the form of pillars, extending from the membrane surface. The anchors are located inside of a perimeter of the cell-growing region of the membrane. In some embodiments, the membrane is treated or formed to facilitate temporary attachment of cells, and later removal of the cells from the membrane to form a cell sheet. In some embodiments, the device has a wall to contain seed cells on the membrane. In some embodiments, the membrane and anchors, or the membrane, anchors and wall, may be integral with each other, for example by being molded together as one casting. In some embodiments, the device has two anchors. In other embodiments, the device has 3 or more anchors.

In some embodiments, the surface of a membrane is patterned. A line between two anchors may be parallel with, normal to, or oblique to a part of the membrane patterned with parallel grooves.

In some embodiments, a device has an interior wall or divider between two anchors or between two pairs of anchors. In some embodiments, the device has an indentation between two or more anchors.

In some embodiments, a device has electrodes. The electrodes may be in, on or near the anchors, or the anchors may also be the electrodes. In some embodiments, the membrane is flexible. In some embodiments, the device is attached to a mechanical stimulation implement.

In some embodiments, a device has a plenum around the anchors. In some embodiments, a device with a plenum around the anchors is connected to another device having a plenum around the anchors.

In some embodiments a device has at least one of magnetic pillar. In some embodiments, the device has a form, for example a hollow fiber membrane, extending between two or more pillars of the device.

This specification also describes a method of growing a cell construct. Cells are grown on a membrane, i.e. a substrate. The cells attach or fuse to each other to form single- or multi-layer precursor constructs. In some embodiments, the one or more layers have an extra-cellular matrix (ECM) containing components produced by the cells. Optionally, the one or more cell layers have openings, i.e. areas without cells internal to a perimeter of the one or more cell layers. In some embodiments, openings are formed by anchors of the membrane passing through the one or more cell layers. At least a portion of the one or more cell layers are released from the membrane thereby forming a cell sheet. The cell sheet is located around a plurality of anchors and cultured whereby the cell sheet remodels to form a cell construct attached to the anchors. Optionally, the releasing step occurs before the locating step. Alternatively, the releasing step overlaps with the locating step, for example in embodiments wherein the anchors pass through the cell sheet. In some embodiments, cells continue to remodel the ECM and optionally, but not necessarily, will grow (i.e. proliferate) such that the cell sheet reorganizes or remodels around the anchors and forms the cell construct. In some embodiments, the cell construct remodels in contact with a form. The cell construct preferably includes ECM components produced by the cells. In some embodiments, remodeling the ECM may include destruction of parts of an existing ECM and re-producing ECM in the form of the new construct.

The method of growing cell constructs is optionally combined with the device. In this case, cells are grown in layers, including an ECM produced by the cells, on the membrane of the device and optionally on parts of the anchors. The perimeter of the layers surrounds a plurality of anchors of the device. In some embodiments the anchors form and pass through openings in the cell layers. A cell sheet is released from the membrane. In some embodiments, release of the cell sheet is accomplished or aided by scraping at least one or more edges of the cell layers from the membrane. Optionally, the edges of the cell layers are detached from a perimeter of the cell layers towards the anchors, followed be a waiting period, followed by detaching the cell layers from between the anchors. In some examples release of the cell sheet is accomplished or aided by use of an environmentally responsive aspect of the membrane. In some embodiments, detachment of the cells happens spontaneously through a force generated and applied by the cells, for example skeletal muscle or heart muscle cells. Cell culture is continued whereby the detached cell sheet remodels around the anchors to form a cell construct of a different shape, for example a fiber or a sheet of a different thickness or shape.

In some embodiments, the cells and their extra-cellular matrix (ECM) may re-align according to tension in the cell construct between the anchors. After a period of incubation, the cell construct becomes more stable than a detached cell sheet that does not remodel in association with anchors. Optionally, the cell construct may be removed from the anchors.

The cell construct may be used while still attached to the anchors, or after being removed from the anchors. In some embodiments, the cell construct may be used for in vitro modeling of tissues or organs, for example in drug discovery. In some embodiments the cell construct, alone or combined with other cell constructs, may form for example an implantable structure or part of an artificial organ, or be used for a non-medical application such as for food, e.g. to provide lab-grown or cultivated meat. In some embodiments, the construct may be decellularized and used as a scaffold or as a source of ECM materials.

In some embodiments, the method described herein produces a cell construct without dispersing the cells in a three-dimensional scaffold or synthetic matrix material. Instead, a cell sheet is produced and then the cell sheet is cultured in association with a plurality of anchors. The cell sheet thereby remodels into a 3D cellular construct with different form factors compared to the precursor cell sheet. Optionally, no exogenous ECM components, or no exogenous biomaterials of any kind, are used to form the ECM. The initial shape of a cell sheet is influenced by the surface of the membrane, but the final cell construct is not limited to the shape of the initial cell sheet. The cell construct may be a sheet of a different shape or thickness, or another shape such as a fiber or rod. The extra-cellular matrix of the cell construct may be produced substantially or entirely by the cells. For example, while additives such as crosslinkers or crowding agents may be used in the process, the ECM of the cell construct may contain 90% or more, or 95% or more, of materials produced by the cells, or the ECM of the construct may be entirely produced by the cells. The cell construct may be in tension between the anchors, which may result in producing a stable and robust construct, having aligned cells and an aligned ECM.

In some embodiments, a method includes releasing one or more cell layers by one or more of scraping the edges of the one or more layers away from the membrane, growing the layers to a thickness that spontaneously detaches from the membrane, and activating a responsive surface of the membrane.

In some embodiments, a method includes providing electrical or mechanical stimulus to a construct.

In some embodiments, a method includes adding a spheroid or organoid to a construct.

In some embodiments, a method includes decellularizing a construct. Optionally, a decellularized construct may be used as a scaffold, as a source of ECM materials or for implantation.

In some embodiments, a construct is used for in vitro modeling or for implantation.

In some embodiments, two or more constructs are cultured in communication through a common media, directly or through a selective membrane.

In some embodiments, a method includes combining a construct with another construct. In some embodiments, a construct comprises cells of a plurality of types. Cells of different types may occupy different lateral portions of the construct or different radial positions of the construct. In some embodiments, a cell sheet is made with different types of cells, followed by forming the construct. In some embodiments, a first construct is made from a first cell sheet, and a second cell sheet is added to the first construct to form a second construct.

In some embodiments, a method includes monitoring passive contraction of a cell construct. In some embodiments, a method includes monitoring a response of a construct to electrical or mechanical stimulation. In some embodiments, a method includes monitoring electrical signal propagation in a construct.

In some embodiments, a construct remodels against a form. Optionally, the form is cylindrical or tubular. In some embodiments, the construct is tubular or has a curved surface.

This specification also describes a membrane, or a membrane and anchors, having a mix of materials. The materials may include elastomeric materials, such as poly (butylene adipate-co-terephthalate) (PBAT), silicone, and urethane rubber. The membrane, or membrane and anchors, may be made by casting two or more of a PBAT resin, a mixture of PBAT and a silicone such as PDMS, a silicone resin and a urethane rubber, in a mold.

This specification also describes a substrate suitable for cell sheet culture comprising anchors. The substrate may have a responsive surface, for example a temperature responsive surface. Alternatively or additionally, the substrate may have a patterned surface. Alternatively or additionally, the substrate may have a porous surface. In some embodiments, the anchors are molded with the substrate, optionally before the responsive surface is functionalized. In some embodiments, the anchors are made separately and attached to the remainder of the substrate, e.g. to the temperature responsive and/or patterned and/or porous surface.

This specification also describes a method of making a cell construct wherein anchors are provided in the proximity of cell layers or a cell sheet. In some embodiments, the anchors pass through the cell layers or cell sheet. In some embodiments, the cell construct is made without the anchors protruding through the cell layers or cell sheet.

This specification also describes a method of making a cell construct comprising making a first cell construct, decellularizing the first cell construct, and adding new cells to re-cellularize the first construct.

This specification also describes a method of cultivating a cell construct comprising locating the cell construct at an air-liquid interface.

This specification also describes a system or method for monitoring a cell construct comprising a camera. In some embodiments, the camera has lighting and filters suitable for fluorescence imaging. In some embodiments, the camera is integrated with a microscope. The camera may allow for observing deformation of an anchor and/or fluorescence of the construct.

This specification also describes a cell culture system or method wherein a first cell construct hanging between anchors is cultivated in a plenum containing a medium. The first cell construct may be in communication with a second cell construct hanging between anchors cultivated in a plenum containing a medium. The cell constructs may communication through the medium directly or via a selectively permeable membrane.

This specification also describes cell constructs, for example cell constructs as described above which may be optionally decellularized as described above, loaded with one or more therapeutic agents such as drugs, genes or cells; to methods of loading the cell constructs; and to methods of using the loaded cell constructs, for example by way of implanting the loaded cell construct in a patient.

### BRIEF DESCRIPTION OF FIGURES

FIG. 1 shows an isometric view of a mold for a cell culture device.

FIG. 2 shows an isometric view of a cell culture device made from the mold of FIG. 1, the device in this example having a non-porous membrane.

FIG. 3 is a schematic representation of method of growing a cell construct showing, Panel A: growing cell layers on a membrane and detaching edges of the cell layers to form a cell sheet; Panel B: culturing the cell sheet, whereby the cell sheet attaches to anchors on the membrane and forms a cell construct; and, Panel C: culturing the cell construct.

FIG. 4 is a schematic representation of another method of growing a cell construct showing, Panel A: growing cell layers on a membrane and detaching edges of the layers to form a cell sheet; and, Panel B: allowing the cell sheet to attach to anchors on the membrane and form a cell construct, wherein the anchors are configured to facilitate removing the construct.

FIG. 5 is a schematic representation of another method of growing a cell construct showing, Panel A: growing cells in layers on a membrane having more than two, e.g. four, anchors; Panel B: detaching edges of the layers to form a cell sheet; and, Panel C: culturing the cell sheet on the anchors of the membrane.

FIG. 6 is a schematic representation of another method of growing a cell construct showing, Panel A: growing one or more layers of a first cell type on a membrane; Panel B: growing one or more layers of a second cell type on the layers of the first cell type; Panel C: detaching edges of the layers to form a cell sheet; and, Panel D: culturing a cell construct derived from the cell sheet on the anchors.

FIG. 7 is a schematic representation of another method of growing a cell construct showing, Panel A: growing one or more layers of a first cell type on a membrane; Panel B: forming a construct of the first cell type on the anchors; Panel C: growing one or more layers of a second cell type on the membrane; Panel D: detaching edges of the layers of the second cell type; and, Panel E: culturing a construct of the second cell type on the construct of the first cell type wherein a cell sheet of the second cell type is wrapped or remodels around the first cell construct.

FIG. 8 is a schematic representation of another method of growing a cell construct showing, Panel A: growing layers of two cell types on a membrane, each cell type surrounding a different anchor; Panel B: detaching edges of the layers as cell sheets; and, Panel C: culturing a construct of having two laterally spaced cell types.

FIG. 9 is a schematic representation of another method of growing a cell construct showing, Panel A: growing layers of a first cell type and a separate layers of a second cell type with a divider between them; Panel B: detaching edges of the layers to form two cell sheets; and, Panel C: culturing a construct of the first cell type and a separate construct of the second cell type.

FIG. 10 is a schematic representation of another method of growing a cell construct showing, Panel A: growing a cell layers against a spheroid and detaching edges of the layers to form a cell sheet; and, culturing a construct including the cell sheet and the spheroid integrated with each other.

FIG. 11 is a schematic representation of three devices having, Panel A: two anchors normal to a pattern of parallel, optionally micron scale, grooves of the surface of the membrane; Panel B: two anchors oblique to a pattern of parallel, optionally micron scale, grooves of the surface of the membrane; and, Panel C: two anchors parallel to a parallel, optionally micron scale, grooves of the surface of the membrane.

FIG. 12 is a schematic representation of a process of applying mechanical stimulation to a cell construct by way of bending or stretching the device.

FIG. 13 is a schematic representation of a process of monitoring passive contraction of a cell construct.

FIG. 14 is a schematic representation of applying cyclical electrical stimulation to a construct and a process of monitoring response of the construct.

FIG. 15 is drawings of devices having, Panel A: electrodes normal to a line between two anchors; and, Panel B: electrodes parallel to a line between two anchors

FIG. 16 is a schematic representation of a process of monitoring electrical signal propagation in a construct.

FIG. 17 shows a photograph of a cell culture device with a magnetic pillar.

FIG. 18 shows photographs of the cell culture device of FIG. 17 with the magnetic pillar in different positions due to a magnetic force that is alternately removed and applied.

FIG. 19 shows two series of photographs of an anchored cell sheet forming a construct on two anchors.

FIG. 20 shows a series of photographs of an anchored cell sheet forming a construct on four anchors.

FIG. 21 shows, Panel A: a scanning electron microscope (SEM) image of decellularized muscle fiber showing aligned ECM components from a top view; Panel B: an (SEM) image of decellularized muscle fiber showing aligned ECM components from a cross section view; Panel C: a photograph of a cell construct growing on an ECM left after decellularizing a sheet hanging between four anchors; and, Panel D: a photograph showing decellularized ECM and a region of recellularized ECM.

FIG. 22 shows, Panel A: an exploded view of a cell culture device with a plenum; and, Panel B: an assembled view of the cell culture device.

FIG. 23 shows a series of photographs showing steps in the formation of a cell construct against a form, in this example forming a hollow tubular cell construct.

FIG. 24 shows a system including a plurality of the culture devices of FIG. 22 connected to each other.

FIG. 25 shows a system including a plurality of the cell culture devices of FIG. 22 connected indirectly through a permeable or semi-permeable membrane.

FIG. 26 shows a device for growing cells having a porous membrane.

FIG. 27 shows development of a cell sheet engineering platform for the biofabrication of self-assembled cell sheets without external triggers or stimuli. a) Culture devices of varying shapes and sizes are fabricated using 3D printed master molds, optionally finished with acetone vapor to partially remove printing patterns. These culture devices are formed by casting and curing PDMS and Ecoflex™ 00-30 and then carefully peeling them off. b) Multiple layers of cells are cultured on top of each other, stimulated to secrete large quantities of ECM, contrary to most 2D culture systems. Sheets are formed by merely scraping the loosely attached cell-ECM layer off the membrane. c) Flexible Ecoflex™ 00-30 pillars protrude from the PDMS membrane, serving as suitable anchors for the delaminated cell sheets. The relative positioning of these pillars to the membrane patterns can guide cell activity in tissue construct formation, thereby fine-tuning their remodeling activities. d) Although unstable independently, the cell sheets detect the presence of pillars and remodel themselves into 3D tissue constructs. This platform is capable of forming complex in vivo-like constructs, such as anchored fibers or sheets, which can be integrated with other scaffold-free constructs such as spheroids.

FIG. 28 shows tannic acid treatment permanently adjusts the hydrophilicity of PDMS without needing surface activation like plasma treatment. a) Effect of tannic acid solution concentration and duration of treatment on the surface contact angle. b) Hydrophobicity of untreated PDMS and PDMS subjected to a 72-hour treatment with a 50 mg/mL tannic acid solution. c) Changes in the elemental composition of the PDMS surface before and after the tannic acid treatment. d) Effect of tannic acid treatment on cell attachment to the PDMS surface, using TC-treated polystyrene as a comparison.

FIG. 29 shows FDM 3D printing enables creation of patterns on culture device membranes to guide cell alignment and cell sheet formation. a) SEM images of patterned and minimally patterned membranes, highlighting the effects of these patterning techniques on cells. Both patterned and minimally patterned membranes have similar cross-sections. b) Membrane featuring concentric and curved patterns, demonstrating cells' capacity to align according to pattern variations. c) A multi-layer cell construct detaches from the membrane as a self-assembled cell sheet, maintaining cell alignment.

FIG. 30 shows guiding cell sheets to form anchored, scaffold-free constructs for in vitro modeling. a) Uncontrolled shrinkage of standalone cell sheets leading to the formation of dense, spherical constructs. b) Introduction of two pillars guides the formation of anchored fibers, with pillar positioning influencing fiber uniformity. Preservation of sheet-like constructs can be achieved through providing c) 4 or d) 6 pillars by forming anchored sheets.

FIG. 31 shows histological analysis of cell sheet-based constructs allows detailed visualization of microstructure and composition. a) Hematoxylin and Eosin (H&E) stain highlights nuclei in blue to dark-purple and the cytoplasm and other structures in varying shades of pink. b) Movat's Pentachrome stain shows collagen and reticular fibers in yellow, muscle in red, elastin in dark black or brown, mucin in blue, and nuclei in black. c) Masson's Trichrome stain with collagen in blue, nuclei in black, and cytoplasm and muscle fibers in red or pink. d) Picrosirius Red stain used in conjunction with polarized light microscopy, where collagen fibers appear bright yellow or green, dependent on fiber thickness.

FIG. 32 shows integration of different scaffold-free tissue constructs for the formation of more complex, in vivo-like structures. a) CAD models for the spheroid biofabrication culture device and phase contrast image showing individual spheroids after 24 hours of incubation as well as their gradual fusion with the cell layer. b) Consolidation of spheroids and their further integration with the multi-layer 2D culture and eventual transition from 2D to scaffold-free fiber, engulfing the spheroid. c) Bright field and fluorescent images demonstrating the retention of the spheroid in its original position and its integration within the anchored fiber.

FIG. 33 shows Air-Liquid Interface (ALI) culture state and submerged cultures were prepared using decellularized SKMC anchored sheets recellularized with A549 cells. a) Bright field and fluorescent images of cells in ALI and submerged states. b) Images of SKMC hanging sheets pre and post decellularization and re-cellularization and the top and side views of anchored ECM sheets.

FIG. 34 shows ECM plays a crucial role in the structure and mechanical integrity of cell sheets and derived 3D constructs. a) SEM views of decellularized standalone sheets and anchored fibers showing ECM arrangement. b) Tensile strength testing of fibers and their decellularized ECM.

FIG. 35 shows the abundant ECM in SKMC cell sheets was confirmed via proteomics analysis and used as a superior coating for cell adhesion. a) Network analysis of ECM-related proteins identified in SKMC cell sheets. Nodes represent proteins, with physical interactions indicated by edges. Edge thickness reflects the confidence level of the interaction: low (0.150), medium (0.400), high (0.700), and highest (0.900). The proteins are grouped into three clusters via k-means clustering, revealing potential protein complexes within the ECM composition (Nodes: 44, edges: 53, average local clustering coefficient: 0.465, PPI enrichment p-value:<1.0e-16). b) Cell sheet-derived ECM (CSdECM) treated well plates demonstrated improved cell adhesion and proliferation for both SKMCs and A549 cells compared to untreated and tissue culture-treated controls.

FIG. 36 shows a network visualization of broader extracellular space components identified from the SKMC sheets. The network represents proteins identified in the ECM space (n=139 of 2270 proteins from cellular component gene ontology analysis). Node shading represent different protein clusters, and line thickness indicates the strength of data support for each interaction.

FIG. 37 shows the results of tensile stress testing of a 5 cm long fiber and its corresponding decellularized ECM fiber.

FIG. 38 shows the effect of treatment with lignin on PDMS wettability.

FIG. 39 is a series of photographs showing the impact of membrane patterns on the attachment and alignment of primary bovine myoblasts.

FIG. 40 is a series of photographs showing the reuse of cell culture devices following a wash with 2-Propanol and autoclaving.

FIG. 41 shows a series of photographs of steps in the formation of a hepatocyte cell construct.

FIG. 42 shows the size of the 3D tissue constructs can be manipulated by controlling the surface area and dimensions of the culture device. In this instance, a longer muscle fiber was fabricated using SKMCs by extending the length of the culture device from 3 cm to 7 cm, which resulted in a fiber that was 5 cm in length. As the width of the device remained constant, the thickness of the fiber stayed the same.

FIG. 43 shows the formation of anchored cell sheet engineering constructs using other cell sheet engineering platforms is facilitated by attaching Ecoflex™ 00-30 pillars to the lid of temperature-sensitive Nunc® UpCell™ Surface cell culture 12 well plates (Sigma-Aldrich, product number Z688797). These pillars are held in place using magnets. Shown are the bottom views of SKMC sheets released at room temperature both without any pillars and with varying numbers and configurations of pillars. In the absence of pillars, the sheets tend to collapse on themselves, while the presence of pillars helps in maintaining stable, defined shapes for the sheets. A single layer of SKMCs was cultured in each well. Following 4 days of differentiation, the pillars were installed and the sheets were detached. The pillars were designed to maintain a distance of 0.1 mm from the well surface, where the cells were cultivated.

FIG. 44 shows the biofabrication of muscle-specific ECM Fibers and VML surgery protocol. a) Schematic representation of the Anchored Cell Sheet Engineering biofabrication process. Primary myoblasts were cultured in multiple layers on patterned PDMS surfaces. Following differentiation and ECM production, the cell sheets were scraped, anchoring to flexible pillars, facilitating further remodeling and maturation of the muscle fibers. These mature fibers were decellularized to leave behind muscle tissue-specific structured ECM. Proper microstructure of the fibers is shown through distribution of laminin as well as aligned components seen in SEM. Scale bars: 200 μm. b) Surgical procedure for creating VML injury model. The left Tibialis Anterior (TA) muscle was accessed through a lateral fascial incision, and approximately 20% of muscle mass was excised 1 cm from the proximal origin. The defect was treated with either test articles (five acellular ECM fibers) or control articles (SIS), sutured in place, followed by fascial and skin closure. In the sham group, the defect remained untreated. Engineered ECM fibers can be fabricated with any length to match the defect size, but their fiber-like form factor provides greater flexibility in matching defect shapes by allowing multiple fibers to be implanted in different regions of the VML injury to meet local requirements.

FIG. 45 shows an automated image analysis pipeline for histological assessment. a) Workflow schematic showing region of interest (ROI) identification in whole slide images (WSI) using QuPath software. The ROI contour and 500 μm grid overlay were exported as SVG files and processed using a custom Python code to generate masked ROI images and subdivide them into analysis tiles. b) Representative segmentation of Masson's Trichrome (segments: “Nuclei/Cytoplasm” light red, “Fibrosis” blue/green, “Muscle” dark red, “Other” weakly stained) and Movat's Pentachrome (segments: “Nuclei/Elastic Fiber” dark brown/black, “Fibrosis” yellow/light brown, “Muscle/Cytoplasm” dark red, “Other” weakly stained) staining each with four segments. c) Segmentation of H&E (segments: “Nuclei” dark purple, “Cytoplasm/Fibrosis/Muscle”, pink/red, “Other” weakly stained) and IHC (segments: “Nuclei” purple, “Target” brown, “Other” weakly stained) each containing three segments. Area coverage percentage for each segment was calculated per tile. Each complete tile is 500 by 500 μm.

FIG. 46 shows a temporal assessment of VML Recovery Across Treatment Groups. a) Representative histological sections from the defect center (largest cross-sectional area, muscle belly) showing progression of tissue remodeling. Test group exhibited gradual defect size reduction from weeks 2 to 8. Control group showed rapid initial shrinkage between weeks 2 to 4, with only minimal inflamed tissue remaining at week 8. Sham group displayed no regenerating tissue after week 2, with only residual inflammation visible. Scale bars: 2 mm. b) Quantitative analysis of tissue recovery. Left: Active site surface area measurements over time (n=5 per group per timepoint); Center: Muscle weight comparison at different time points, showing 20% reduction in control and sham groups versus native muscle, while test group maintained native-equivalent mass at week 8; Right: in vivo functional assessment of muscles by measuring peak isometric force after percutaneously stimulating peroneal nerve. Peak isometric force at maximum stimulation frequency showed 91-92% recovery in sham and control groups versus 77% in test group compared to contralateral muscles. Data are presented as box plots showing median, interquartile range, and individual data points. Lines connect mean values between consecutive time points for each condition. For Area, Weight, and Force measurements across experimental conditions and time points, one-way ANOVA was performed followed by pairwise Welch's t-tests between conditions within each week. P-value<0.05 was considered significant, n.s. not significant, *<0.05, **<0.01.

FIG. 47 shows histology and IHC images of test, control, and sham groups. Temporal changes in the active treatment sites showed distinctive patterns when different staining types and targets were evaluated. These images were further quantified using a combination of QuPath and a custom Python script. The start in Test, Week 2, H&E shows areas of test article that were not populated by any cell type at the beginning of the process while no such large areas were found in control group. The start in Control, Week 2, H&E shows the fat deposition deep into the implanted SIS subject observed early on in some of the control subjects, indicating severe fibrotic response. Not only were high levels of CD31 expression observed in test group, but also lumen-like structures were formed as early as week 2, shown by the arrows. Sham group showed much lower cell densities that were uniformly distributed, mostly fibrotic and inflammatory in nature across the defect area. Scale bar: 2 mm.

FIG. 48 shows quantified observations from histology images using tile level data. The percentage area of each segment was recorded across different tiles of histological images from all animals in each condition and time point. a) Hematoxylin and Eosin (H&E) staining provided reliable detection of nuclei across all conditions, revealing higher cell numbers in the test and control compared to other groups. b) Masson's Trichrome and c) Movat's Pentachrome staining methods effectively detected fibrotic regions within the active treatment area. The test group exhibited persistent but gradually diminishing fibrotic tissue presence in both staining groups. Tile level information was used for developing the plots. Data are presented as box plots showing median (center line), interquartile range (box), and individual data points. Lines connect mean values between consecutive time points for each condition. A mixed-effects model was used for statistical analysis to account for multiple tile measurements within each animal, followed by pairwise Welch's t-tests between conditions and weeks with Bonferroni correction for multiple comparisons. P-values for all the comparisons are included as heatmaps in FIG. 55.

FIG. 49 shows temporal changes in IHC markers shown a) using tile-level data, and b) through unitless representation of slide-level data with the Target Prevalence Index (TPI). Various markers for vascularization (CD31), immune response (CD68), fibrotic response (FSP1), muscle regeneration (Desmin), and muscle and fibrotic tissue ECM components (Laminin and collagen I) demonstrated distinct temporal patterns across different treatment groups compared to native tissue. While IHC groups effectively detected specific targets, nuclei detection was compromised in certain cases as target staining occasionally masked the nuclei. TPI was calculated by normalizing the area coverage value for each IHC target to the total nuclei area detected in Hematoxylin and Eosin staining, providing a unitless index for more meaningful temporal comparisons between different conditions and native tissue. The test condition maintained elevated presence of endothelial cells (CD31) and demonstrated progressive formation of vascular lumens over time, while the control group exhibited significantly lower vascularization. Immune cell (CD68) presence gradually decreased to near-native levels in the test group, whereas the control group maintained high levels at weeks 2 and 4. Fibrotic cells (FSP1) demonstrated patterns similar to immune cells in both test and control groups. Desmin index remained lower than native tissue but had inched closer to native tissue by week 8. While collagen levels were sustained in test group, its levels significantly increased in control. Laminin levels showed a marked decrease in test group and only modest decrease in control. Slide level information for each animal was generated and used here by calculating mean and standard deviation from all the tiles for that animal. Data are presented as box plots showing median, interquartile range, and individual data points. Lines connect mean values between consecutive time points for each condition. For tile-level and slide-level data a mixed-effect model and a weighted two-sample t-test were used, respectively. P-values for all the comparisons are included as heatmaps in FIG. 56.

FIG. 50 shows differences between control and test groups in recruiting muscle progenitor cells and new myofiber formation shown using Masson's Trichrome and Desmin staining. While quantification at both tile- and slide-level showed more Desmin positive cells in the control group at all time points, a clear transition between native muscle and implanted article was detectable with no proper myofiber formation. At the same time, the test group showed a transition zone where native muscle, the implanted ECM fibers, and both newly formed scattered myofibers (shown by the arrows) and the actively remodeling fibrotic tissue were present. Lack of cohesion between these myofibers could have caused the lower force recorded in the test group. Arrows point to scattered newly formed myofibers in the implant location. Scale bar: 500 μm.

FIG. 51 shows a protein-protein interaction network analysis of extracellular matrix components visualized using K-means clustering (k=3) generated using the STRING database. Line thickness indicates the strength of data support for known associations.

FIG. 52 shows protein-protein interaction network analysis of cell-ECM adhesion proteins visualized using K-means clustering (k=3) generated using the STRING database. Line thickness indicates the strength of data support for known associations.

FIG. 53 shows the detection of three zones in the muscle in QuPath software, actively remodeling treatment site, intact muscle and transition zone separating the two. The transition zone could be muscle tissue being damaged due to inflammatory response to the damage or new partially regenerated muscle. As it couldn't be confidently assigned to either, only the actively remodeling site was assessed in the current study.

FIG. 54 shows a force-frequency relationship of dorsiflexor muscles under different experimental conditions. Native tissue shows highest force production, with peak forces reaching ˜1300 mN at maximal stimulation frequency (150 Hz). Error bars represent standard error of the mean (SEM). n is 6, 6, 5, and 4 for contralateral muscle, test, control, and sham groups, respectively.

FIG. 55 shows a comprehensive statistical analysis for histology image analysis. A mixed-effects model was employed followed by a pairwise t-tests with unequal variance assumption (Welch's t-test) between groups at different timepoint. P-values were adjusted for multiple comparisons using the Bonferroni correction method.

FIG. 56 shows a comprehensive statistical analysis for IHC images using slide- and tile-level values. For tile-level data the same mixed-effects model was employed. Slide level data (mean of all tile values for each slide) were used with a weighted two-sample t-tests with Bonferroni correction implemented to account for standard deviations calculated from tile level data.

### DETAILED DESCRIPTION

The term “cell sheet” has been used in the art to refer to a sheet-like collection of cells with preserved cell-to-cell junctions. In some embodiments, the cell sheets described herein also have an extra-cellular matrix (ECM) produced by the cells. The ECM is initially produced while the cells are attached (e.g. adhered) to a culture surface though it may be modified by the cells later. Depending on context, the term “cell sheet” usually also indicates that the collection of cells has been released from the culture surface that was used during the process of forming the cell sheet. An exogenous three-dimensional (3D) scaffolding (for example a hydrogel) is not provided during formation of a cell sheet. 3D scaffoldings cause cell suspension and/or aggregation while the cells are not attached to a two-dimensional (2D) surface which is a distinct process can result in cell-to-cell junctions and/or cell-to-ECM attachments that include the exogenous scaffolding material.

A cell sheet conventionally contains a monolayer of cells, with multi-layer or 3D cultures produced by stacking detached monolayer cell sheets together. In contrast, in some embodiments described herein, a multi-layer collection of cells is formed prior to releasing the cells from the culture surface. When released, a multi-layer cell sheet is formed without using cell sheet stacking methods. The released cell sheet has a thickness of more than one cell and so may be considered a form of 3D culture although the sheet may still be considered a 2D object. The term “cell construct” is usually used herein to refer to a remodeled form of the initial cell sheet. In some embodiments, the remodeled form is still sheet-like, though it may become thicker than the initial cell sheet. In other embodiments, the remodeled form has a different shape, for example the shape of a fiber. Growing multiple layers of cells on top of each other prior to release from a culture surface mimics the natural organization of many tissues, potentially leading to more physiologically relevant constructs.

The tissue engineering approach described herein does not rely on external scaffolds or on exogenous extra-cellular matrix (ECM) materials (such as collagen, fibrin, Matrigel, fibrinogen, thrombin or mixtures of such materials) to provide a material part of the ECM. In scaffold dependent methods a culture cavity is typically filled with a hydrogel scaffold including exogenous ECM material and cells suspended in the hydrogel. In some of these methods, the cells are placed in the culture cavity in a liquid dispersion and may briefly rest on the bottom of the culture cavity but rise by contraction soon after the liquid materials form a gel. In alternative methods, the cells are dispersed and suspended in a hydrogel bioink before being placed in the culture cavity. In either of these methods, the cells aggregate without being attached to the bottom of the culture cavity. In addition, significant amounts of the exogenous ECM materials are incorporated into the ECM of the resulting tissue.

In contrast, methods described herein are scaffold-free and may be essentially (e.g. 98% or more) or entirely exogenous ECM material free. Many cells will adhere to surfaces with certain levels of hydrophilicity, but in some embodiments treating the culture surface to promote the attachment of certain cells that only adhere to certain ECM materials (such as collagen, vitronectin or laminin) may include coating the surface (with or without hydrophilic treatment) with an ECM material. In some embodiments, coatings of other materials, for example polydopamine or poly-I-lysine, may also be useful in allowing certain cells to adhere to a culture surface. Coating the culture surface with an ECM material typically involves exposing the culture surface to a dilute solution of the ECM material for a period of time and then washing the culture surface before adding the cells. The ECM material is thereby present as a coating (e.g. individual molecules forming a molecular brush-like structure) rather than a layer. The ECM surface coating is usually degraded by enzymes produced by the cells, but a very small amount of the ECM surface coating may remain in the cell sheet. However, this ECM surface coating has no material role in providing the structure or composition of the cell sheet ECM. Instead, the cells produce their own ECM and the cell-produced ECM is the primary material used in cell-to-ECM attachments. In some embodiments, this can simplify the production process and potentially improve the biological relevance of the resulting construct.

In some embodiments, the culture surface is patterned and optionally made of an elastomeric material. The use of a patterned surface may induce ECM production while the cells are attached to the culture surface and the multi-layer collection of cells is forming. The patterned surface also facilitates growing multiple layers of cells on top of each other before detachment, possibly by creating a 2.5D sense for the cells.

In some embodiments, the multi-layer collection of cells can be detached, or detachment can be initiated, by simple scraping. The scraping does not need to be preceded by adding an enzyme such as dispase. Optionally, as discussed above, in some embodiments adhesion of some cell types to the culture surface (e.g. membrane) is improved with a coating that could include an ECM derived material (such as laminin or vitronectin) but this coating is used to enhance cell-to-surface adhesion without leaving a material amount of the coating material in the cell sheet. Detachment by scraping also does not rely on techniques such as surfaces responsive to temperature or other stimuli or ultrasound irradiation. Detachment by scraping also does not involve manipulating magnetically labelled cells, which are typically held against an ultralow-attachment surface or hanging in the middle of a culture system by a magnet and not attached to a surface to begin with. Initiating or completing detachment by simple scraping can be efficient and can be achieved with minimal damage to the collection of cells.

In some embodiments, the hydrophilicity of the culture surface has been modified, for example by a polyphenol surface treatment, to encourage initial cell adhesion (i.e. attachment). The hydrophilicity treatment may be used in combination with treating the culture surface with an ECM component coating, or other coatings, to further enhance attachment of cells to the culture surface as mentioned above. The use of ECM components in this way to promote attachment of certain cells to the culture surface to aid in forming a cell sheet (i.e. to allow the cells to form cell-to-cell junctions and cell-produced ECM while attached to the culture surface) is distinct from the use of ECM components to provide a 3D scaffold in which the cells initially aggregate while suspended in the ECM components.

In some embodiments, the culture surface (alternatively called a membrane) has one or more pillars. The pillars act as anchors for the detached cell sheet and facilitate remodeling or maturation of the cell construct. In some embodiments, there are multiple spaced apart pillars. In some embodiments, the pillars are made of an elastomer. The pillars may resemble pillars used in hydrogel-based methods wherein cells aggregate while not attached to a surface. However, in the method described herein the cells attach to the culture surface and, over time, produce ECM and establish cell to cell junctions while attached to the culture surface and in the absence of a hydrogel or porous scaffolding. While some of the cells might attach to the base of the pillars adjacent the culture surface, it is not hypothesized that the cells otherwise interact with the pillars before the cell sheet is released. For example, it is not hypothesized that the cells orient themselves around the circumference of the pillars under internal tension while still attached to the culture surface. Instead, the cells appear to cover the entire culture surface evenly, and the cell sheet when released is two-dimensional or sheet-like having essentially the size and shape of the entire culture surface less the area occupied by the pillars. In some embodiments, the resulting structure is more cell-dense and/or contains a more physiologically relevant ECM than constructs produced in a hydrogel or other scaffolding or by first making a cellular ring. Optionally, in a method of making a membrane, T-shaped or capped pillars can be molded integrally with the culture surface in a hard mold. The hard mold does not need to be dissolved to release the pillars, such that the mold can be re-used.

In some embodiments, the methods described herein result in increased ECM content, and optionally more physiologically relevant ECM, relative to prior methods.

In some embodiments, three-dimensional biological structures or tissues are created without the use of exogenous 3D structural such as scaffolds. In this approach, cells are the primary building blocks and are manipulated to self-organize and produce their own extracellular matrix (ECM) to form the desired tissue structure. This technique relies on the cells' innate ability to communicate, produced ECM components, and form direct cell-to-cell junctions, ultimately leading to the formation of complex tissues or organs without the need for artificial scaffolding materials. In some embodiments, the method is characterized by an absence of exogenous structural materials such as scaffolds during the initial tissue formation process and/or by reliance on cell-cell junctions and cell-produced ECM for structural integrity. The method provides the potential for more physiologically relevant tissue formation by minimizing foreign materials, particularly ECM materials. In some embodiments the method does not involve various complex methods to promote cell aggregation or cell sheet formation, such as hanging drop cultures, pellet cultures, magnetic particles (either attached to the cells or added to the culture media), or specialized surfaces responsive to for example temperature, electrical stimulation, photo exposure, or ROS diffusion, or a layer or sheet of exogenous ECM or other exogenous material that remains with the cell sheet.

In the context of tissue engineering and biofabrication, a hydrogel is usually a three-dimensional network of hydrophilic polymers that can absorb and retain large amounts of water while maintaining its structure. Hydrogels are commonly used as scaffolds, bioinks, or supportive matrices in creating 3D cultures due to their biocompatibility and structural similarities to natural extracellular matrices. Key characteristics of hydrogels in tissue engineering may include one or more of: a high water content, typically 70-99% by weight; a cross-linked polymer network structure; biocompatibility and often biodegradability; tunable mechanical and chemical properties; ability to encapsulate cells and bioactive molecules; natural (e.g., collagen, alginate) or synthetic (e.g., polyethylene glycol) origin; and use to provide structural support and a conducive environment for cell growth and tissue formation. In many biofabrication techniques, hydrogels serve as a temporary scaffold that supports cell growth and tissue development. However, in a scaffold-free approach as described herein, such exogenous hydrogels are intentionally avoided to allow for more cell-driven tissue formation.

In some embodiments, the initial attachment of the cells to the membrane endures, depending on the cell type, for at least one week, and optionally for about the first 2 to 4 weeks of the process. During this time, the cells produce material amounts of ECM. When the cells are scraped or otherwise detached from the culture surface they form an ECM-rich sheet-like structure that may be already relatively stable compared to cell sheets produced by some other processes. This initial cell sheet is sufficiently robust so that it can migrate to the pillars for further remodeling and maturation into other stable shapes. Essentially all (e.g. 98% more) of the ECM in the resulting cell construct is produced by the cells.

A system and method described herein uses an optionally scaffold-free cell sheet to form, optionally with self-assembly, a 3D cell construct. In one exemplary use, to help generate physiologically pertinent in vitro models the system and method may facilitate control over cellular behavior, notably extracellular matrix (ECM) production and remodeling. In some examples, scaffold-free muscle fibers and lung epithelial cell cultures at the air-liquid interface, among other cell types and applications, are examined as representative examples. In some embodiments, various scaffold-free models, each optionally with their tissue-appropriate form factor, can be fused to construct assembloids reflecting organ-level structure and function. In some embodiments, different tissues are connected using a fluidic system to simulate in vivo systems. In some embodiments, ECM-rich anchored constructs, derived from cell sheets and subsequently decellularized, are used to create ECM-based scaffolds. In some embodiments, this cell sheet-derived ECM (CSdECM) is solubilized for use in 2D models, offering a tissue-relevant scaffold material with a well-defined composition. The systems and methods described herein may alternatively be used in regenerative medicine, for example to create implantable materials.

The system and method employ cell sheets as building blocks and supplies the cell sheets with anchors, enabling in some embodiments the recreation of diverse tissues with their corresponding structures and form factors. Recognizing the anchors' guidance, cells remodel ECM-rich cell sheets into functioning tissue avatars that may accurately mirror in vivo tissue, which is useful for example for in vitro modeling applications or to create implantable materials. In some embodiments, muscle fibers and air-liquid interface systems for lung epithelial cells, both devoid of exogenous membranes or scaffoldings, are generated. Optionally, the ECM component from a construct can be harvested as CSdECM, which is suitable for use as a scaffold for other cell types. The CSdECM can also be solubilized for use as a coating or a hydrogel to replace animal-derived hydrogels, offering a reproducible and controlled alternative. Optionally, individual tissue models can be generated and interlinked to create a more complex representation of human body physiology.

A device and process for growing cell sheets is described in our U.S. application Ser. No. 17/882,693, Silicone-Based Membrane Surface Chemistry and Topography Control for Making Self-Assembled Cell Sheets with Cell Alignment and Positioning, filed on Aug. 8, 2022 and issued as U.S. Pat. No. 11,718,830, and in International Application No. PCT/CA2023/050779, Device and Method for Making Cell Sheets, filed on Jun. 7, 2023, which are incorporated herein by reference. This application describes, for example, making patterned molds for example by way of 3D printing, casting a resin for example of PDMS on the mold, treating the mold for example with a polyphenol to make it hydrophilic, growing a cell sheet on the membrane, and removing the cell sheet from the membrane for example by scraping and/or pulling. In at least some of the examples described herein, these devices and methods are adapted to produce a cell construct. Other devices and methods of growing cell sheets may alternatively be adapted for use with the devices and methods described herein. For example, cell sheets may be grown on membranes having responsive surfaces such as polystyrene grafted with N-isopropylacrylamide (PIPAAm). In other examples, cells sheets are grown on a membrane and detached using an enzyme to preferentially digest parts of the ECM and the cell sheet to membrane junction. In another example, cell sheets may be grown as described in International Publication Number WO 2022/000086 A1, Self-Assembled Cell Sheet Constructs and Methods of Making Thereof, described further above.

As used herein, the term “membrane” refers to a substrate suitable for supporting a cell sheet. Porosity is optional, but in some embodiments the membrane is a bulk material. A membrane made of a bulk material is non-porous to bulk liquid flow, or at least capable of supporting a liquid media over the cells, although a membrane made of a bulk materially may optionally be permeable to oxygen or other gasses. In other embodiments, the membrane is made by a process that produces pores in the membrane or the membrane is made from a mesh or other structure with openings. Openings are preferably less than the size of cells being grown on a membrane such that cells do not pass through the membrane. Membranes may be made of various thermoplastics or thermosets. The membranes may be rigid or elastomeric.

Membranes can be formed, for example, by molding, casting or extrusion. Some suitable materials are biocompatible epoxy resins, acrylic resins, polyurethane (PU) resins, and thermoplastic resins. Epoxy resins, such as those sold under the ARALDITE trademark, are thermosetting polymers that can be used for casting and molding applications. They are often used in conjunction with a hardener component. However, their biocompatibility needs to be thoroughly evaluated, as some epoxy resins can potentially release cytotoxic compounds. Acrylic resins, such as polymethyl methacrylate (PMMA) or poly(2-hydroxyethyl methacrylate) (PHEMA), can be used for casting or extrusion processes. They are transparent and can be biocompatible. PHEMA, for instance, has been used in applications such as contact lenses and is hydrophilic, which helps with cell adhesion. Polyurethanes are versatile materials that can be used for casting, molding, and extrusion. Their properties can range from very soft and flexible to hard and rigid, depending on their formulation. Some PUs have been used in medical and cell culture applications due to their good biocompatibility. Thermoplastic resins such as polystyrene (PS), polycarbonate (PC), and cyclic olefin copolymer (COC) can be used for injection molding or hot embossing. PS is the standard material for cell culture dishes and flasks due to its biocompatibility and cell-adhesive properties. PC and COC, can also be used for cell culture applications after suitable surface treatment or coating.

Elastomeric membranes can include, for example, thermoplastic elastomers based on poly(butylene adipate-co-terephthalate) (PBAT), urethane rubbers formed for example by reacting a polyol with an isocyanate, and silicones. Optionally, commercially available materials from Smooth-On (e.g. Smooth-On PMC 121/30 WET rubber), Polytek, EcoFlex and Reynolds Advanced Materials can be used for cell culture related applications. Elastomeric membranes made of a silicone may include, for example, PDMS. However, elastomeric membranes typically have limitations due to their hydrophobicity, such as inappropriate cell attachment, agglomeration, and detachment, which may be improved with surface treatment as described herein. PDMS is used as an exemplary membrane in many examples herein, but other materials may be used. PBAT is molded with PDMS in many examples herein, but other materials may be used.

Other suitable silicone based membrane materials include silicone urethane resins, silicone acrylate resins and silicone epoxy resins. Silicone urethane resins are a class of materials that combine the flexibility and ease of processing of silicones with the toughness and durability of urethanes. An example of this type of resin is QSil 553, sold by Quantum Silicones. This is a two-part, platinum-catalyzed, liquid silicone urethane that is often used for molding applications. Silicone acrylate resins combine the properties of silicones with those of acrylates. They can be formulated to provide a variety of properties including high flexibility, weatherability, and adhesion to a variety of substrates. An example is BR-5141, sold by Gelest Inc. Silicone epoxy resins combine the properties of silicones and epoxies. They can provide high temperature resistance, good adhesion, and low shrinkage. Examples include the KER 7100 and KER 7200 series sold by Shin-Etsu.

Optionally, a membrane may be coated or treated with an environmentally responsive material, for example a thermally responsive material. Environmentally responsive materials may transition between a hydrophobic or hydrophilic state based on an environmental factor such as temperature, pH or ionic strength. In some examples, environmentally sensitive materials include a hydrogel or polymer brush that expands or contracts in response to a change in an environmental factor. After a desired number of cell layers are grown on the membrane, a change in the environmental factor is used to cause a change in the environmentally responsive material to cause the cell layers to separate from the membrane or to assist in separating cell layers from the membrane. However, in some examples an environmentally responsive material is not used.

In a method described herein, a cell sheet is formed from cells grown on a membrane. Optionally, the membrane is reusable. The membrane may have topographical patterns on at least part of its surface. Depending on the cells being grown, or a target tissue being created, different patterns can be used to influence one or more aspects of cellular behavior such as cell attachment, alignment, differentiation, extracellular matrix (ECM) production and remodeling.

One method for making a membrane involves first making a mold. For example a mold may be 3D printed using a polymer such as polylactic acid (PLA), acrylonitrile butadiene styrene (ABS), or polyvinyl butyral (PVB). In the next step a resin suitable for cell culture, preferably with temporary cell attachment, is cast in or over the mold. Once the resin solidifies, the membrane can be removed from the mold. Topographical features of the mold are mirrored on the membrane. In the case of a 3D printed mold, topographical features created inherently by the 3D printing process are mirrored on the membrane. After 3D printing a mold, extra features can be made on the mold surface, for example by using laser engraving. Different parameters such as intensity of the laser source as well as time of treatment can be used to create different feature shapes and sizes on the surface of the mold. It is also possible to reduce or eliminate some or most of the patterns on the surface of the mold. For example ABS can be treated with acetone vapor while PVB can be treated with 2-propanol vapor. Timing of the treatment as well as the partial pressure of the vapor can be used to determine to what extent as well as what feature sizes (i.e. nano-, micro- or meso-scale features) of the patterns will be removed. The membranes that are created on vapor treated surfaces may be less patterned, or less deeply patterned, than membranes formed on untreated molds. Alternatively, a mold may be machined with topographical features. Alternatively or additionally, the membrane may be machined with topographical features. For example, a PDMS membrane may be laser engraved.

Suitable membrane resins include but are not limited to silicone-based polymers such as polydimethylsiloxane (PDMS) and poly(butylene adipate-co-terephthalate) (PBAT). PBAT is commercially available in formulations with varying stiffness, for example as sold under the trademark Ecoflex™. It is also possible to use a blend consisting of different ratios of PDMS and PBAT, or to cast PDMS into one part of a mold and PBAT into another part of a mold.

Once the resin solidifies, it can be removed from the mold. For some resins, the hydrophilicity of the surface of the membrane is optionally adjusted for cell culture, for example to provide a hydrophilic surface. In some examples, the membrane is treated with a polyphenol such as lignin, or a tannin such as tannic acid, to make the surfaces hydrophilic and suitable for cell attachment. The concentration of a polyphenol solution in water, and duration of contact with the membrane, can be used to create different hydrophilicities suitable for different cell types. After this surface treatment, membranes can be sterilized, for example by washing with 70% ethanol solution, UV treatment, and/or autoclaving before cells are grown on the membranes. After the membrane is sterilized, further treatment with sterile solutions of different ECM components such as vitronectin, laminin, or fibronectin can optionally be performed if cells require specific recognition moieties for attachment.

Once the membranes are prepared, the process of sheet formation starts with seeding the cells, optionally progenitor or stem cells, on the membrane. In some examples, the cells are differentiated by applying a differentiation media. It is also possible to use other cell types. A few days after cells adhere and optionally differentiate on the membrane, more cells are optionally added. The additional cells adhere to the first layer of cells, and optionally fuse with them while differentiating. This may be aided by the presence of patterns on the membrane that give a sense of 3D environment to the cells as compared to the 2D nature of a completely flat surface. The step of adding cells can be performed one or more times, e.g. 1 to 10 times, so that a multilayer cell construct is created. A medium used to grow cells, and optionally to differentiate cells, can also contain components that increase the cells' ECM production, which can improve the integrity of a cell sheet. Different elements can be added to the medium at different time points of the process, depending on the cells' state of differentiation or growth.

Once cells have grown and optionally differentiated properly and have produced enough ECM, the cells can be detached from the membrane to form a sheet that includes both the cells and their ECM. In some examples, a cell sheet detaches spontaneously after reaching a certain thickness. In other examples, a cell sheet is scraped off the membrane. One process of sheet removal involves scraping the edges of the membrane to lift off the sheet and then lifting the whole sheet by grabbing the released edges and slowly pealing it off. If cells were aligned due to the presence of patterns on the membrane, they will maintain their directionality in the detached cell sheet.

A membrane may be substantially flat when considered on a scale larger than any patterning. The membrane may be surrounded by a wall adapted to retain a liquid medium and/or loose cells over the membrane. Alternatively, a membrane without walls may be inserted into a container, for example a dish or tray, to retain liquid and/or cells over the membrane.

In a device and method described herein, a membrane is provided with anchors or otherwise associated with anchors. The anchors may be protuberances extending from the planar surface of the membrane. The anchors support the cell sheet after the cell sheet has detached from the planar surface of the membrane. The anchors inhibit uncontrolled agglomeration of the cell sheet. The anchors may also provide the cell sheet with support useful for maintaining the integrity of the cell sheet as it remodels into a cell construct of a different shape. Various numbers of anchors may be positioned in various locations or in various distributions on top of the membrane.

In some embodiments, the anchors are integral with the membrane. For examples, a mold may be configured to produce a membrane with the anchors in a single casting. In some examples, a mold such as a 3D printed mold is made with anchor shaped cavities. The resin is cast on the mold, thereby producing both the membrane and the anchors.

In some embodiments, after solidifying the membrane is peeled off to form a cell culture device having three components: 1. the membrane used for cell sheet culture, 2. a wall, optionally used for holding the membrane as well as keeping the culture media and loose cells on the membrane, and 3. the anchors, optionally in the shape of pillars. All of these components can be made out of the same resin, different resins, or different ratios of different resins mixed with each other. Different anchors can have different shapes, sizes, and even different chemical and mechanical properties.

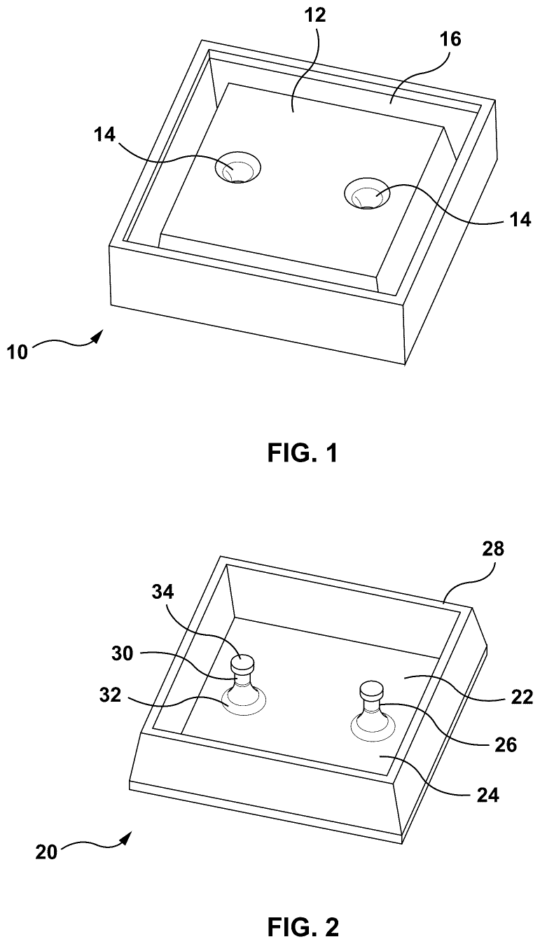

FIG. 1 shows a mold 10. The mold 10 in this embodiment is a single (i.e. unitary) 3D printed solid. The mold 10 has an open top and resin poured into the mold 10 has a free surface. Optionally, a mold 10 may be made in two or more parts. A mold may also be enclosed such that resin is injected into the mold and has no free surface.

FIG. 2 shows a device 20 made by casting a resin in the mold 10. As shown, the device 20 is inverted relative to its orientation while being molded. The mold 10 has a platform 12, which defines an upper surface 22 of a membrane 24 of the device 20. The mold 10 also has two indentations 14 that define anchors 26, which are protuberances extending from the membrane 24. The mold 10 also has a moat 16, which defines a wall 28 of the device 20. The mold 10 and device 20 are drawn to scale. In this example, the device 20 is approximately square and the membrane 24 is about 30 mm by 30 mm wide. Alternatively, the membrane 24 may have another shape, for example round, oval, diamond-shaped or rectangular, with a length of sides or a diameter for example in the range of 10-100 mm. The wall 28 may be, for example, 5-10 mm high. In alternative embodiments, the anchors 26 may be made of another material, for example a ceramic or plastic, and potted into the membrane 24 during casting or added to the membrane 24 after casting. The anchors 26 of a device may be the same as each other or different. In some embodiments, the anchors 26 are rigid while in other embodiments at least one of the anchors 26 is flexible. Variations in the stiffness of the anchors 26 may allow the device 20 to better approximate the in vivo environment of a particular tissue type.

The anchor 26 in this example is in the form of a pillar. The anchor 26 in the embodiment shown has a conical section 32 adjacent the membrane 24, a cylindrical section 30 and a cap 34. The conical section 32 is optional but may help with guiding or lifting a cell sheet away from the membrane 24 such that the cell construct eventually attaches to and/or hangs from the anchors 26. The cell construct typically forms around and/or attaches to the cylindrical section 30. The cap 34 is also optional but inhibits further lifting of the cell construct upwards beyond the cylindrical section 30. Without the cap 34, in some cases the cell construct may bend the anchors 26 or simply remodel or lift itself upwards until the cell construct is released from the anchors 26. The cap 34 may be spherical, or a cylindrical disc larger in diameter than cylindrical section 30. In applications where the cell construct will be removed from the device, the cap 34 may be omitted or smaller than the cap 34 shown.

In the embodiment shown, the mold 10 is made by Fused Deposition Modeling (FDM) 3D printing. FDM printing uses a thermoplastic filament that fuses to adjacent filaments to form a solid structure. The filament inherently produces patterns on the mold 10 may then be mirrored on the upper surface 22 of the membrane 24 or other parts of the device 20. These patterns (e.g., parallel patterns) on the membrane 24 can be used for aligning the cells. Alternatively, other materials, for example thermoplastics or metals, may be used to create a mold 10. Other techniques of creating patterns such as CNC machining or laser engraving may be used to create patterns in a mold 10.

Different sizes or resolutions of patterns can have different effects on cell alignment. In some examples, FDM 3D printers used for printing the mold 10 have nozzle sizes of 0.25 mm, 0.4 mm, or 0.6 mm and the printing speed is 20-100 mm/s. Smaller nozzle sizes and lower printing speeds may also be used and will create smaller features that might perform better in cell alignment for some cell types.

3D printed filaments are typically thermoplastic polymers, such as PETG, ABS, or PVB (polyvinyl butyral). The molds 10 may be optionally treated to alter the patterns. For example, ABS molds may be treated with acetone vapor and PVB molds may be treated with isopropyl alcohol vapor to melt the surfaces of the mold 10. This changes the depth, resolution or smoothness of the patterns on the mold 10 and consequently changes the patterns on the membrane 24. In some embodiments, treatment times with the vapor phase of a solvent of a polymeric filament 3D printed mold 10 ranged from about 0 minutes to about 30 minutes. After the vapor treatment, the mold 10 can be heat treated at temperatures 20-60° C. for 1-12 hours in order for the solvent to evaporate from the surface of the mold 10 and for the polymer to solidify.

Independent of the exact thermoplastic filament material used for 3D printing the molds, three levels of features are created on the surface of the mold 10. Meso scale patterns can have 100-500 μm spacing between adjecent features. These patterns can have some moderate effect on cell alignment. Micron scale patterns, thinner features spaced 5-50 μm apart, are typically most influential in aligning the cells. Nano scale patterns, features smaller than 1 micometer (<1 μm), create surface roughnesses that induce cells to secrete higher amounts of ECM components. These features together create a 3D-like environment for cells growth and function that facilitates cells behavior closer to in vivo conditions compared to completely flat membranes. Using larger sizes of printer nozzles or higher speeds of printing will shift the feature sizes to larger ones in the mentioned ranges.

To produce the device 20, a liquid resin, for example an elastomer solution, may be cast into the mold 10 and solidified. The mold 10 may be heated, for example to a temperatures ranging from 25-90° C. for a time period of 1-24 hours to cure the resin. Curing at higher temperatures will reduce the time required for solidifying the resin. The curing temperature may be chosen based on compatibility of the material used for 3D printing the mold to avoid distorting the features of the mold.

The upper surface 22 of the membrane 24 provides the substrate for growing a cell sheet. The wall 28 retains a suspension of cells in liquid media over the membrane 24. In the embodiment shown, the membrane 24, the anchors 26 and the wall 28 are contiguous or integral, although they are not necessarily the same material. For example, membrane 24 may be made from silicone resin such as PDMS while the wall 28 and anchors 26 are made of PBAT. The wall 28 and the anchors 26, or each individual anchor 26, may be made of different grades of PBAT having different stiffness. The PBAT is poured into the indentations 14 and moat 16 and optionally allowed to gel but not completely set, and the PDMS is then added to cover the PBAT and the platform 12. In one example, anchors 26 are made with Ecoflex 30, a flexible PBAT resin, the wall 28 is made out of Ecoflex 50, a more rigid PBAT resin, and the membrane 24 is made out of PDMS.

EcoFlex includes a family of biodegradable, thermoplastic elastomers based on poly(butylene adipate-co-terephthalate) or PBAT, and also some silicone based resins. The chemical structure of PBAT consists of alternating units of adipate ester and terephthalate ester derived from 1,4-butanediol, adipic acid, and terephthalic acid. PBAT is synthesized via copolymerization of these monomers.

Many elastomeric materials, including silicones and many non-silicone-based membranes, are hydrophobic materials. Hydrophobic materials are generally unsuitable for cell attachment and tend to cause cells to form aggregate clumps rather than flat layers. Membrane fabrication to impart surface topography as described above may improve cell sheet formation. Further, the surface of elastomeric or other membranes are optionally modified by chemical treatment to make them more suitable for cell attachment.

In some examples, elastomeric membranes, for example silicone membranes such as PDMS, are treated with an aqueous solution of a polyphenol. The polyphenol may be plant based, for example tannic acid or lignin. The polyphenol may be applied to the membrane without additional cross-linkers or compounding agents.

In some embodiments, the treatment may include treating the membrane with 10-200 mg/mL of tannic acid, optionally 25-100 mg/mL. The treatment may be done generally at ambient or room temperature. The treatment period may be about 6 hours to 4 days, or 1-3 days. The tannic acid solution may be essentially unbuffered, for example, the aqueous solution may have a pH of 7.5 or less or 7.0 or less. Optionally, the tannic acid solution contains only tannic acid and water. The tannic acid is applied to the membrane without additional cross-linkers or compounding agents, for example no iron is present. Without intending to be limited by theory, dihydroxyphenol or truhydroxyphenol groups of the lignin may be able to adsorb to the surface of PDMS or other elastomers even without surface activation of the membranes. After optional treatment with a caustic solution, as described further below, the membrane may also have functional groups (e.g. silanol groups) available for chemical binding. However, surface activation, for example by plasma treatment, is not required. Further, additional cross-linking of the polyphenol (e.g. with iron (III) or polyethyleneimine, or beyond any crosslinking that may be present in the natural molecule) is not required. In some examples, a cell growth substrate is produced that has essentially only the membrane material and attached polyphenol molecules, wherein the polyphenol molecules are not bound to each other except via the membrane.

In some embodiments, the treatment may include contacting the membrane with a solution of about 0.1-6 mg/mL, or 1-4 mg/mL of lignin. The treatment may be done generally at ambient or room temperature. The contact period may be about 6 hours to 4 days, or 1-3 days. The tannic acid solution may be essentially unbuffered. Optionally, the tannic acid solution contains only tannic acid, water and a water-miscible solvent, for example methanol or another alcohol.

Lower or higher concentrations of polyphenol can be used for this purpose as well, but the timing of the treatment should be adjusted accordingly. In some embodiments, different solutions containing tannic acid and lignin can be used one after the other.

In order to improve the attachment of tannic acid, lignin or another polyphenol to the surface of the membrane, the membrane can be pretreated with a caustic solution, for example an aqueous sodium hydroxide solution. Alternatively, the membrane may be pretreated with an oxygen, atmospheric air, or carbon dioxide plasma. Optionally, the membrane is not pre-treated with either the caustic solution or plasma.

After the polyphenol treatment (e.g. with tannic acid and/or lignin), the membranes may be washed to remove residues. For example, the membranes may be washed with deionized water at least twice to remove the residues. Then, the membranes may be used immediately for cell culture or can be stored at room temperature for a time period of a few weeks or in a refrigerator for a longer time period.

For sterilization purposes, before starting the polyphenol treatment, the membranes can be washed with about 70% ethanol and subjected to UV afterwards or they can be autoclaved. The polyphenol (i.e. tannic acid and/or lignin) solutions can be sterile-filtered using about 0.2 μm syringe filters. The treatment may be done inside a biosafety cabinet to avoid possibility of contamination.

In some examples, PDMS membranes were not pre-treated to activate their surfaces, for example with a caustic solution or plasma. Three concentrations of each of tannic acid (25, 50, and 100 mg/mL) and lignin (1, 2, and 4 mg/mL) aqueous solutions were used with either 24 or 72 hours of treatment. All treatments but for lignin 1 mg/mL for 24 hours produced hydrophilic surfaces. For both tannic acid and lignin, increasing the concentration or duration of treatment decreased contact angle. In cell growth experiments, membranes with contact angles in a range of about 60-87 degrees have been used to grow cell sheets. However, better results are obtained with lower contact angles near 60 than with contact angles near 87. Further, literature suggests that contact angles of 55, or even down to 20, may be useful. Accordingly, the concentration and duration of treatment are optionally chosen to produce a contact angle of 87 degrees or less, 85 degrees or less, 80 degrees or less, 75 degrees or less or 70 degrees or less. The concentration and duration of treatment are optionally chosen to produce a contact angle of 20 degrees or more, 40 degrees or more, 55 degrees or more or 60 degrees or more. The preferred contact angle may also vary with cell type or surface topography of the membrane. Treatment with 50 mg/mL solution of tannic acid for 72 hours has produced high quality cell sheets using a variety of cell types and with smooth or textured PDMS membranes.

As described herein, a “cell sheet” is an optionally scaffold-free or self-assembled sheet-like construct including cells and an ECM that they have secreted, or a portion of such a sheet-like construct. Cell attachment to other cells and to their ECM is preferably preserved in the cell sheet and therefore the cell sheet can be maintained independent of a membrane or surface to adhere to. Since the ECM is preserved in a cell sheet, if multiple sheets are stacked on top of each other, they can bind and fuse to one another. Alternatively or additionally, the cell sheet may be remodeled into a 3D cell construct.

Elastomeric or other membranes, with or without patterns, and optionally treated with a hydrophilic agent such as lignin and/or tannic acid, can be used for cell sheet formation. The membrane provides a surface for cell adherence and cells attach to the membrane at the beginning of the process. Over time, cells proliferate and start adhering to each other and form strong cell-cell junctions. The cells also secrete their own ECM, which they then adhere to.

As cells establish attachments to each other and/or their ECM, their attachment to the membrane weakens. Once the attachment of cells to each other and their ECM becomes stronger than their attachment to the membrane, cells and their ECM may detach spontaneously or can be detached physically, for example by scraping. It is also possible to make a cell sheet detach by scratching the edges of the membrane and shaking it to help cells detach as a sheet by applying shear force. The cell sheets are also strong enough to be grabbed and pulled off of the membrane surface using a tweezer or similar means. Once pulled off from the membrane surface, cells will maintain their alignment for a period of time due to the high density of the cells and high ECM content in the cell sheets that initially prevents them from changing their alignment. For example, the detached cell sheet may be attached to anchors and remodeled to form a 3D cell construct attached to the anchors.

Once the cells cover the surface of the membrane and produce ECM, it is also possible to add more cells that grow on the initial layer of cells in order to create a multilayer construct. This can be performed multiple times to make a thicker construct, but if it is done more than a certain number of times, depending on the cell types and species up to 10 times, the attachment between cells of different layers becomes stronger than attachment of the first layer to the membrane, which can result in spontaneous detachment of the cells.

Optionally, cells can be induced to secrete more ECM components by treating them with components, such as ascorbate family members, or by creating macromolecular crowding by addition of elements such as high molecular weight PEG or carrageenan with proper concentrations to the media. It is also possible to crosslink the ECM secreted by cells prior to addition of more cells to increase stability of the cellular constructs. Lower concentrations of tannic acid or lignin (e.g. less than 0.1 mg/mL tannic acid or less than 10 μg/mL lignin) for short amounts of time in serum-free media to avoid agglomeration of media components can be used for crosslinking ECM components. The timing of treatment can be in a range from about 5 minutes to about 100 minutes depending on the crosslinker type and its concentration prior to addition of the new cells. Multiple cell layers, as well as optionally more ECM content and crosslinking, can result in a sheet with better mechanical integrity that can help with handling the sheets and moving them from one vessel to another after delamination. Additionally, multiple cell types may be used either in each layer or with different cell types on different layers.

In an exemplary method, a membrane is placed in a dish or a device 20 is used. On Day 1, growth media is added to a depth of about 3-5 mm over the membrane. Enough cells are added to over the membrane to about 60-90% or 75-85% confluence. The cells are incubated, for example at 37° C. or the physiological temperature of the cell animal, for 2 days. On Day 3, the cells will have grown to form a completely confluent first (single cell) layer. The growth media is refreshed, i.e. removed (e.g. by aspiration) and replaced with fresh media. In the case of stem cells, the fresh media may be a differentiation media. For stem cells, differentiation and fusing to adjacent cells typically happen together. For other cell types that adhere (rather than fuse) to each other, the fresh media may be more growth media.

On Day 4, cells (typically the same or a similar number of cells as were added on Day 1) are added on the first layer of cells. On Day 5, the media is refreshed, with either new growth media or new differentiation media for stem cells. On Day 6, a second layer has formed and new cells (typically the same or a similar number of cells as were added on Day 1) are added on the second layer of cells. On Day 7 the media is refreshed, with either new growth media or new differentiation media for stem cells. On Day 8, a third layer has formed and new cells (typically the same or a similar number of cells as were added on Day 1) are added on the third layer of cells. On Day 9 the media is refreshed, with either new growth media or new differentiation media for stem cells. In this example, the cell sheet will have four layers. Optionally, more or less, i.e. 2 or 3 or 5 or more, layers may be produced. On Day 11, and every two days thereafter, the media is refreshed again. The cells continue to produce ECM and the cell sheet becomes more robust. On Day 18, an edge of the cell layers may be scraped from the membrane to form a cell sheet at least above the edges of the membrane. Optionally, the layers may be completely removed (e.g. scraped and/or pulled) from the membrane to produce a detached cell sheet. This example describes a typical process but the schedule of cell and media addition or various other steps may be varied.

The description of growing cell sheets above is optionally a first phase in the process of growing a cell construct. In an example using the device 20, after one or more cell sheet layers have formed on the membrane 24, the membrane 24 is scraped to detach the cell sheets. Optionally, edges of the membrane 24 are scraped to detach a cell sheet around the periphery of the membrane 24. The cells are incubated, with further media exchanges are required, and cells may continue lifting off the membrane 24 on their own, or cells still attached to the membrane 24 may become easier to be removed. Portions of a detached cell sheet may also begin wrapping around the anchors 26 and pull attached cells from the membrane 24. Once fully detached from the upper surface 22 of the membrane 24 and attached only to anchors 26, the cells may continue to remodel into a new construct. Depending on the properties of the cells and the sheet that they have formed, as well as the shape and size of the anchors 26, the cells can form different shapes of constructs around the anchors 26. For example, a 2-anchor system may allow a cell sheet to roll up and form a fiber-like construct between the anchors 26 or on both sides of the anchors 26. A 4- or 6-anchor system can be used in order to prevent the cell sheet from rolling and to maintaining a sheet-like form, although a cell sheet suspended between anchors 26 may be thicker than the initial cell sheet and is considered to be a 3D construct. With further incubation, the cells in the hanging constructs will continue to remodel their ECM and realign themselves and therefore the mechanical and structural properties of the construct will change, usually shown by increasing mechanical properties. Unlike stand alone (i.e. unanchored) cell sheets that may show rapid and uncontrolled agglomeration if left without anchors, the anchored constructs show a more controlled remodeling and slower restructuring pace. After a period of time, for example 48 hours, the cell constructs may be stable even if removed from the anchors 26. In some examples, the constructs are substantially or completely free of exogenous scaffold and/or other exogenous biomaterials. The constructs can be used for different applications, including drug discovery and in vitro modeling of different physiological and pathological conditions or for implantation. The constructs can be formed in different sizes and shapes for different applications.

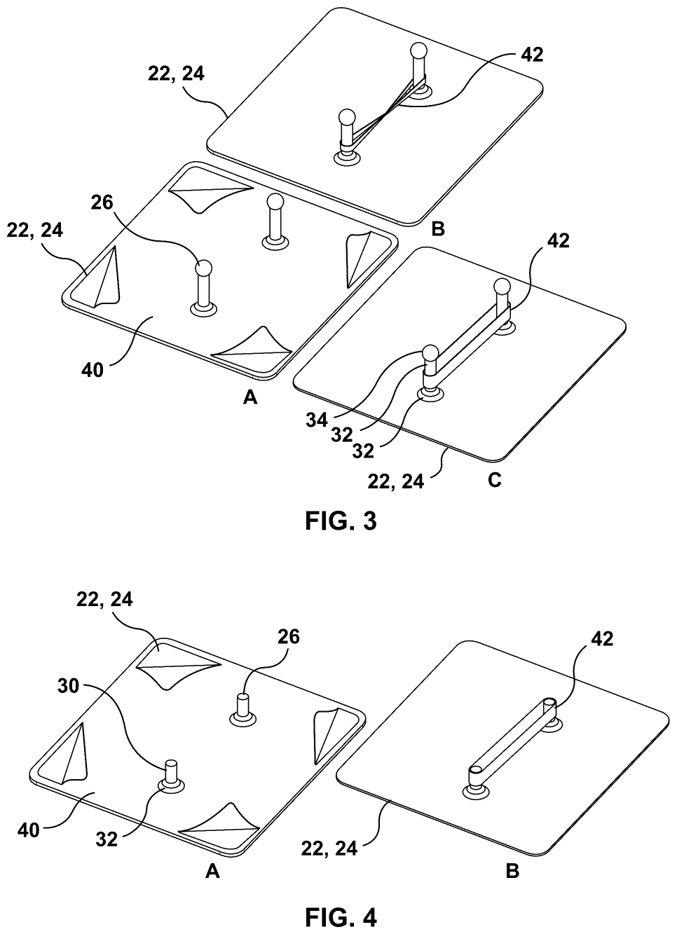

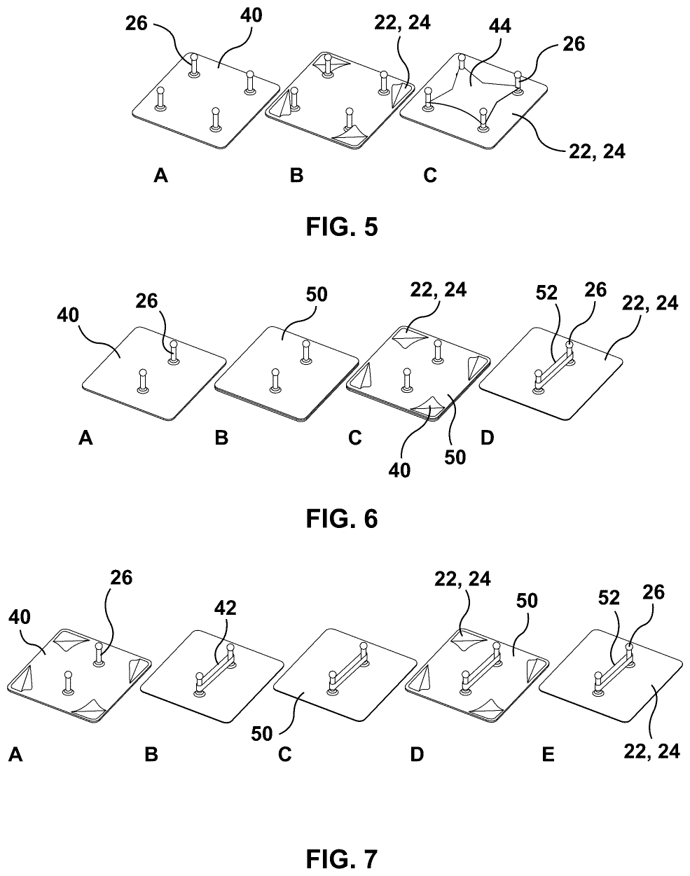

Referring to FIG. 3, one or more cell layers 40 are grown on an upper surface 22 of membrane 24 with two anchors 26. The cell layers 40 may also grow on some parts of the anchors 26 but at least some parts of the anchors 26 create and protrude through openings in the layers 40. Optionally, at least the edges of the layers 40 are scraped to detach them from the membrane 24 (Panel A), and by one method or another the layers 40 detach from the upper surface 22 of the membrane 24 and form a cell sheet. The cells re-attached themselves to the cylindrical section 30 of anchors 26 and form a construct 42 (Panel B), in this embodiment a fiber, suspended from the anchors 26. With continued cultivation, the construct 42 may increase or decrease in size (Panel C). In some embodiments, mechanical properties of the construct 42 may improve. FIG. 4 shows a similar process but the anchors 26 do not have caps 34. The construct 42 may extend over the top of the anchors 26. This construct 42 is easier to remove from the anchors 26 than the construct 42 in FIG. 3. Referring to FIG. 5, one or more cell layers 40 are grown on an upper surface 22 of membrane 24 with more than two anchors 26, in this embodiment four anchors 26 (Panel A). The layers 40 are detached from the upper surface 22 of the membrane 24 (Panel B). The cells re-connect themselves to the cylindrical section 30 of anchors 26 and form another construct, in this embodiment a remodeled or 3D cell sheet 44 (Panel C), suspended from the anchors 26.

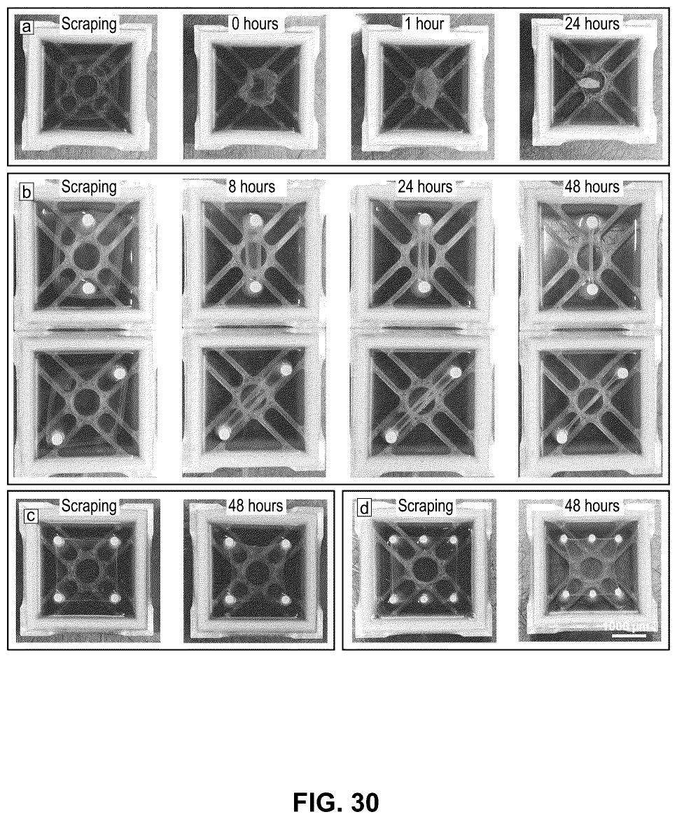

When cells are detached from the surface that they have been adhered to, either as single cells or as cell sheets, they will begin remodeling their cytoskeleton and change their structure and morphology due to loss of their anchorage to the surface. It is shown for example in the top line of FIG. 30 that if no other anchor is found, cell sheets will continue shrinking until they turn into an agglomerate. However, when there is another anchor available, for example in the form of a pillar, the cell sheet will hang on to the anchor and apply a force to it. This force lifts the sheet and causes remodeling, for example into a fiber if two anchors are used or into another sheet if three or more anchors are used. The presence of a conical base 32 helps with guiding the shrinking force in lifting the construct (e.g. sheet or fiber) from the membrane 24 such that it can hang in the middle (vertically) of the device 20 above the membrane 24. Eventually the only attachment of a construct 42 is to the anchors 26.

Once the constructs 42 detach from the membrane 24 and hang between the anchors 26, cells start producing more ECM and remodeling the construct 42 in order to increase its integrity and mechanical properties. The longer the cells remain hanging, the better the integrity of the construct 42 becomes.

If the constructs 42 are released from the anchors 26 shortly after separation from the membrane 26, usually sooner than 48 hours after separation, they may continue to shrink and crumple but not as fast as cell sheets that never hung on the anchors 26. If the constructs 42 are kept on the anchors 26 for a sufficient time, typically at least 48 hours or more, even after taking the constructs 42 off of the anchors 26, they will retain their morphology with minimal further shrinking.

The precise behavior and the time required to form a stable construct 42 may vary with cell type. For example, if fibers made out of muscle cells, they can apply more force, as muscle cells do, and can shorten the fiber significantly even if they have been hanging on the anchors 26 for a long time. Longer time on the anchors 26 however will result in more ECM production and remodeling which makes the fibers stronger so that detached muscle fibers can resist this contractile force for a period of time.

Referring to FIGS. 6-10, constructs with multiple cell types, optionally with controlled and predefined positioning, can be formed. In one method, different cell types can be grown separately on top of each other. As shown in FIG. 6, one or more layers 40 are grown with a first cell type. One or more second layers 50 having a second cell type are grown on top of layers 40. After the layers 40, 50 are detached, a multi-layer cell sheet is formed and wraps around the anchors 26 with one cell type on the inside of the construct and another cell type on the outside to produce a concentrically separated multi-cell type construct 52. As shown in FIG. 7, it is also possible to grow one or more layers 40 of the first cell type and form their corresponding sheet around the anchors to form a construct 42. One or more second layers 50 of a second cell type are then grown on the membrane 24. A cell sheet of the second cell type is later detached and wrapped around the construct 42 that is already hanging between the anchors 26. This also results in a concentrically separated multi-cell type construct 52.

An exemplary use for such multi-cell type constructs 52 could be an in vitro model for the blood-brain barrier with neuronal cells on the inside and the endothelial cell on the outside of the formed construct.

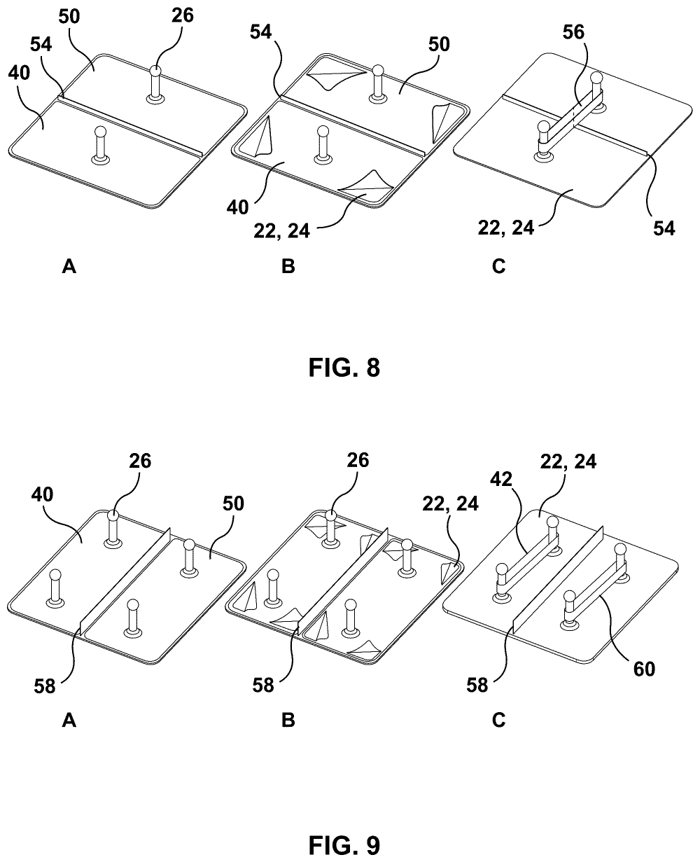

Referring to FIGS. 8 and 9, it is also possible to form constructs with different cell types in different locations. As shown in FIG. 8, a divider 54, optionally molded into membrane 24, separates two anchors 26 from each other. Two different cell types are grown on each side of the divider 54. The divider 54 may be, for example, 1-1.5 mm high. In the first step of the process, cells are added to each side of the divider 54 in low volumes of media such that media from one side of the divider 54 does not over flow to the other side. After cells adhere to the surface 22 of the membrane 24, more media is added to cover the whole surface of the membrane 24. At this point, cells will first cover the membrane 24 and have the ability to slowly grow over the divider 54. Once cells from either side reach the cells from the other side, cells start forming cell-cell junctions with each other, or fuse with each other in the case of muscle cells. This will prevent the cells from moving further to the other side but will create a complete coverage of the surface of the membrane 24. Cells growing on top of the divider 54 form a continuous growth layer. At this point, optional scraping, sheet formation, and anchoring can happen similar to before to form laterally separated multiple cell type constructs 56. These constructs 56 can be used for applications such as neuromuscular junction where neuronal cells and skeletal or heart muscle cells form two different sides of the multi-cell construct 56. In other embodiments, there are two or more dividers 54 and a multi-cell construct 56 has two or more junctions between different cell types.

In some applications different tissues have indirect contact with each other through paracrine effect. In this case cells in each tissue secrete different signaling molecules that reach the other tissue through a shared medium. To model this type of system, as shown in FIG. 9, two sets of anchors 26 are separated by a wall 58. The wall 58 may be a separate part potted into the membrane 24 or integrally molded into the membrane 24. The wall 58 may be made out of a resin not suitable for cell attachment. Different cell types will be grown on each side of the wall 58. A first construct 42 with a first cell type and a second construct 60 with a second cell type may be indirectly in communication with each other through shared medium on top of the membrane 24. Optionally, the first construct 42 and the second construct 60 are selectively put in communication with each by raising the surface of the medium above the top of the wall 58 or separated from each other by lowering the surface of the medium below the top of the wall 58. In another option, the wall 58 has an opening such that a first construct 42 and a second construct 60 are created and always in communication with each other through the opening. In another option, the wall 58 includes a selectively permeable membrane. The selectively permeable membrane prevents or inhibits some components of the medium or cell products from being transferred across the wall 58. In some embodiments, a device has two or more walls 58 and three or more sets of anchors 26 separated by the walls 58. In some embodiments, the same construct 42 is grown in two or more compartments, each compartment defined at least in part by a wall 58.

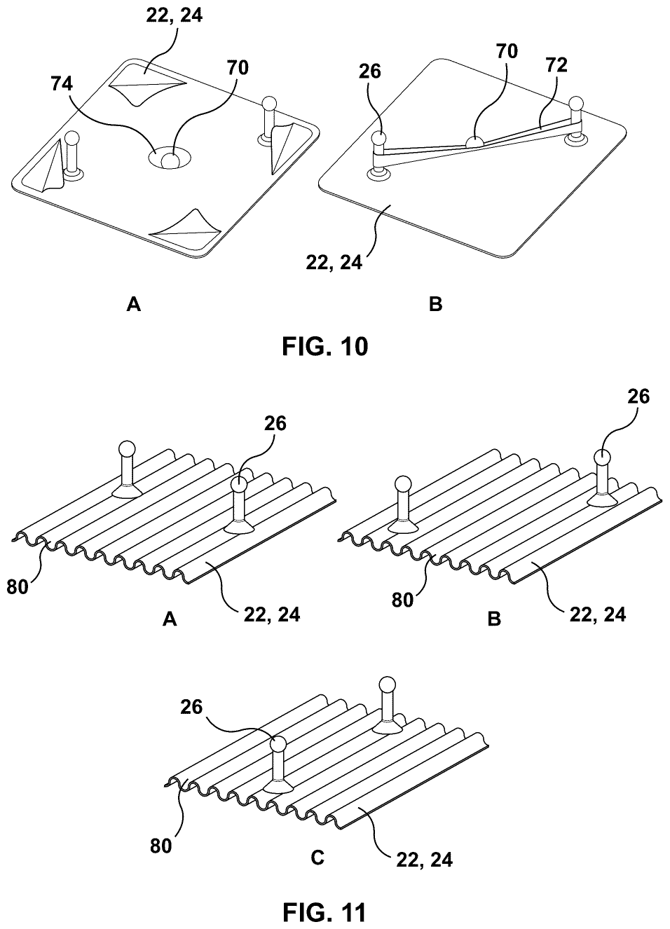

It is also possible to integrate different 2D and 3D constructs, either scaffold-based or scaffold-free, with the sheets grown on the membrane 24. In one example shown in FIG. 10, a cellular spheroid 70 formed by a technique such as hanging drop or another technique is integrated with a cell sheet remodeled into a fiber. For this purpose, a small depression or indentation 74 is made on the surface 22 of the membrane 24 in a desired place. After layers of cells are grown on the membrane 24, one or more spheroids 70, with similar or different cell types as to those grown on the membrane 24, can be positioned in the depression 74. The depression 74 will keep the spheroid 70 in place until it integrates with the cell layers. Optionally, at this point another cell layer can be grown on top of the previous layers to also cover the spheroid 70. The sheet formation and detachment process can continue similar to before and eventually compound construct 72, in this embodiment including a fiber engulfing a spheroid, can be formed between the anchors 26. Optionally, organoids may be used instead of spheroids 70.

One exemplary form of such a compound construct 72 includes neuronal cells for the spheroid 70 and muscle cells for the fiber. Neuronal cell outgrowth can be guided along the fiber and towards the direction of the anchors 26.

In some embodiments, cells are added on top of a hanging sheet construct 44 in order to create a multi-layer and multi cell type construct.

Referring to FIG. 11, the direction of patterns or grooves 80 on the membrane 24 relative to the position of the anchors 26 can define how cells are positioned in the final constructs that are formed around the anchors 26. Positioning of the anchors 26 as well as the 3D mold printing patterns for the grooves 80 can be selected to provide the configurations shown in FIG. 11 or other configurations.

Cell sheets contain high amounts of ECM. The anchored constructs however further increase ECM content and maturity by allowing guided remodeling of the construct and its components. Optionally, it is possible to decellularize such constructs using detergents such as Triton X-100 and Tween-20 and use the remaining ECM constructs as scaffolding for other applications such as regenerative medicine and in vitro modeling. These constructs can be used either as cell-free scaffolds or recellularized constructs for in vivo applications.

A construct may be decellularized while still attached to the anchors 26. Constructs such as sheets and fibers hold on to the anchors 26 by wrapping themselves around the anchors 26. These constructs are made out of cells and their ECM and these two components are entangled with each other, so both ECM and cells are wrapped around the anchors 26. Decellularization is optionally performed by replacing culture medium with detergents. The detergent solution will remove the cells from the constructs and then only ECM components will remain, while still hanging between the anchors 26.

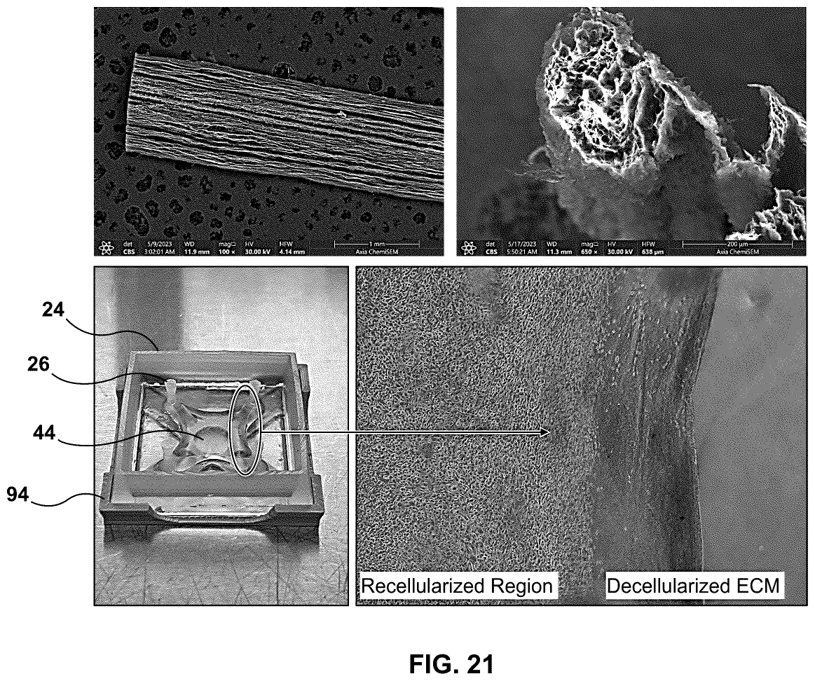

FIG. 21 shows scanning electron microscopy (SEM) images of decellularized muscle fibers that shows aligned ECM components from top and cross-sectional view.

Optionally, a construct such as a sheet form construct 44, may be decellularized for applications such as skin regeneration. Optionally, a decellularized construct 42, for example a sheet form construct 44, is recellularized, for example with skin or lung cells. In some embodiments, an Air-Liquid Interface (ALI) culture system is created by only submerging the bottom of a construct with a medium while exposing the top side to air (FIG. 21, Panels C and D). This may create more in vivo like conditions for some cells, for example skin cells or lung cells, that are exposed to air and a liquid medium in vivo.

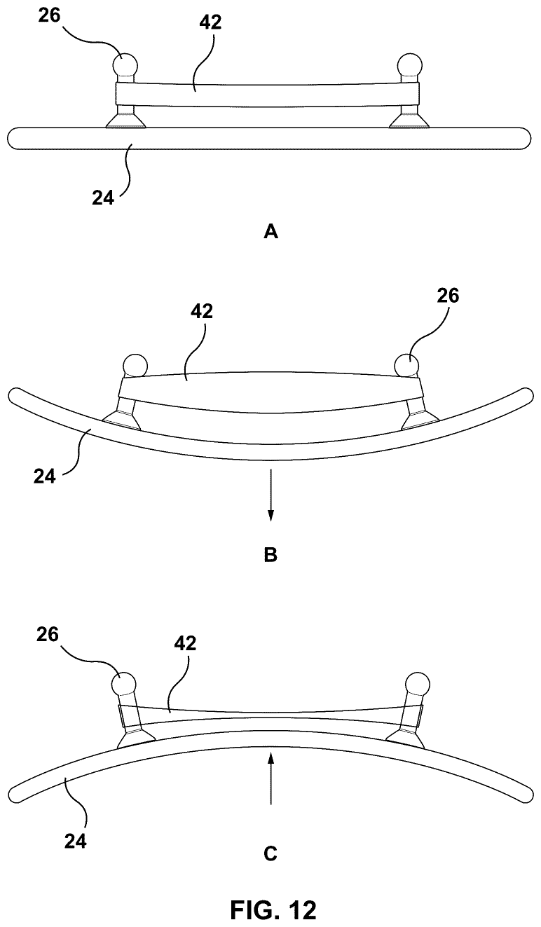

Referring to FIG. 12, once constructs 42 are formed around the anchors 26, the formed constructs 42 can be actuated by applying mechanical deformation to the membrane 24 that will be transferred to the constructs 42. For example, the construct 42 may be stretched, compressed or twisted. One example is through application of air pressure or vacuum to the bottom membrane 24. The membrane 24 can also be stretched or compressed. This could be used for creating a dynamic environment for different cell types that are exposed to such stimuli inside the body including but not limited to skeletal and heart muscle tissues, bone, ligament, and tendon tissues as well as endothelial and epithelial cells. Alternatively, a membrane 24 may be maintained in a deformed position.

Optionally, mechanical stimulation can be provided by way of a magnetic pillar 26. A magnetic pillar 26 can be made with a composite of a resin and magnetic nano- or micro-particles. By controlling the weight ratio of the magnetic particles to the resin, the mechanical properties of the pillar and its magnetic behavior can be adjusted to create a pillar that is flexible enough to be bent by an external magnetic force. This can be used for implementing mechanical stimulation or testing mechanical properties of the formed cell constructs both in anchored fiber and sheet formats. In order to reduce any magnetic shielding effect of the resin wall, a side of the cell culture device 20 can be made with a thinner wall 28. The magnet may be permanent magnet that is moved relative to the magnetic pillar. Alternatively, the magnet may be an electromagnet. By applying different electrical signals, for example altering the polarity or strength of the electromagnetic field, different modes of mechanical stimulation can be applied to the cell construct.

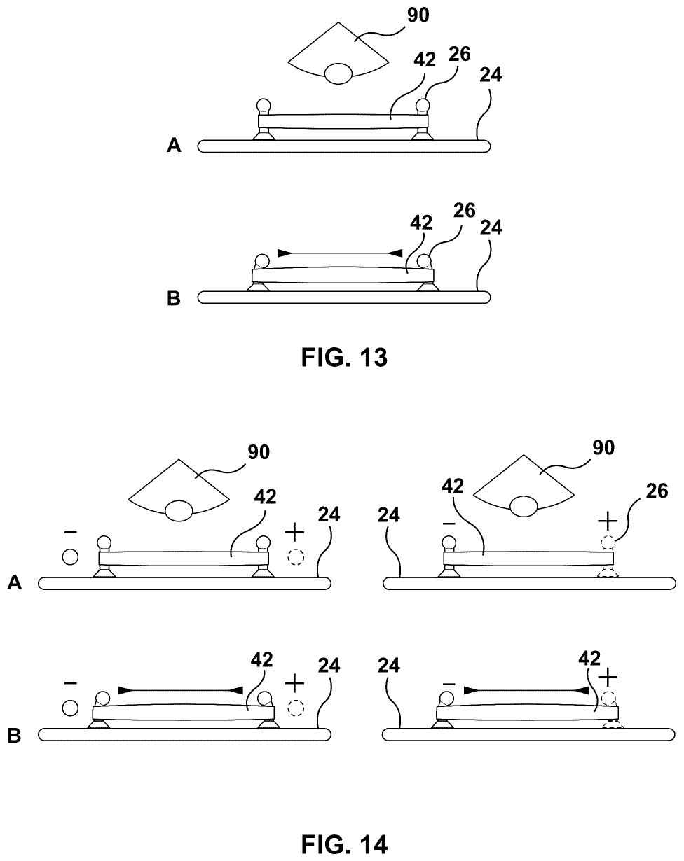

Cell activity can also cause deformation of the anchors 26. By measuring the amount of deformation, and since the mechanical properties of the anchors 26 are known or the relationship between force and deformation can be experimentally obtained, the force generated by the constructs can be measured using a camera 90 to observe anchor 26 deformation as in FIG. 13. Alternatively, another form of distance measuring device, or a strain gage or other sort of force measuring device, may be used. Shape, size, and mechanical properties of the anchors 26 can be controlled to create different amounts of deformation in response to cellular force or to resist the force that is generated by the cells. It is also possible to have one thinner and/or more flexible anchor 26 while the other anchor 26 is thicker and/or made out of a stiffer material. This could result in most of the deformation occurring on the more flexible anchor 26, which may increase the accuracy of the measurements or simplify the measurements since only one anchor 26 is tracked. Measurement of the force generated by the cells can be used for example as a marker for cell construct 42 maturation, to determine the response to an external stimulus, or to test integrity of the cell construct 42 under the effect of different drugs.

Electrical stimulation can also be used for guiding cellular behavior or inducing certain functions for different cell types such as neuronal, skeletal and heart muscle, and bone cells. For example, electrical stimulation may encourage differentiations of stem cells or mesenchymal cells. The force generated by the cells in response to electrical stimulation can optionally be measured through measuring the deformation of the anchors, for example through imaging with a camera 90 as shown in FIG. 14. Applications of such system includes testing integrity of the cell construct under the effect of different drugs. As also indicated in FIG. 14, the electrical stimulation may also be applied and removed at different frequencies. A low frequency may stimulate twitch contraction while a high frequency may stimulate tetanic contraction. The amount of deformation observed may vary with the stiffness of the pillars 26 and the frequency of the stimulation.

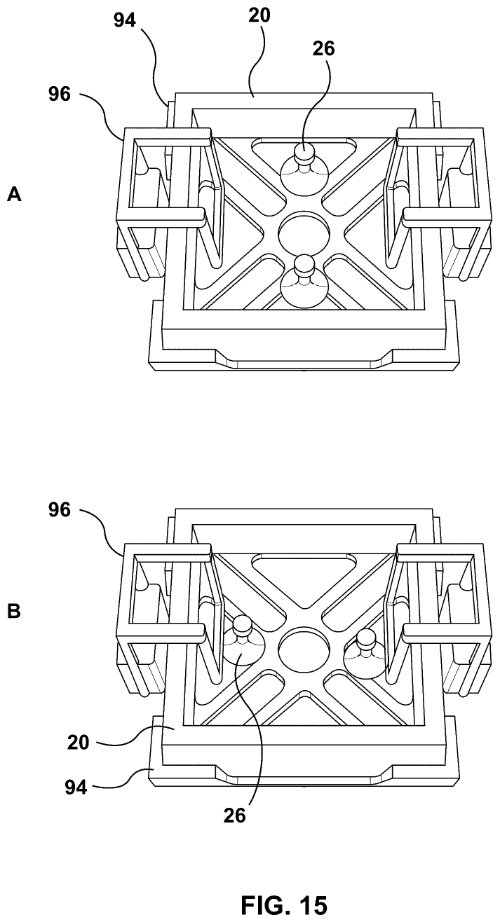

In order to deliver the electrical stimulation to the cells, electrodes 96 can be added to the device 20, either before or after the constructs are formed around the anchors 26, as in FIG. 15. FIG. 15 also shows different ways that electrodes 96 can be positioned compared to arrangements of the anchors 26. Alternatively, the anchors 26 themselves can be used as electrodes for example by coating the anchors 26 with a conductive material or by using a conductive material to construct the anchors 26. Optionally, device may combine a conductive anchor 26 with an external electrode 96.

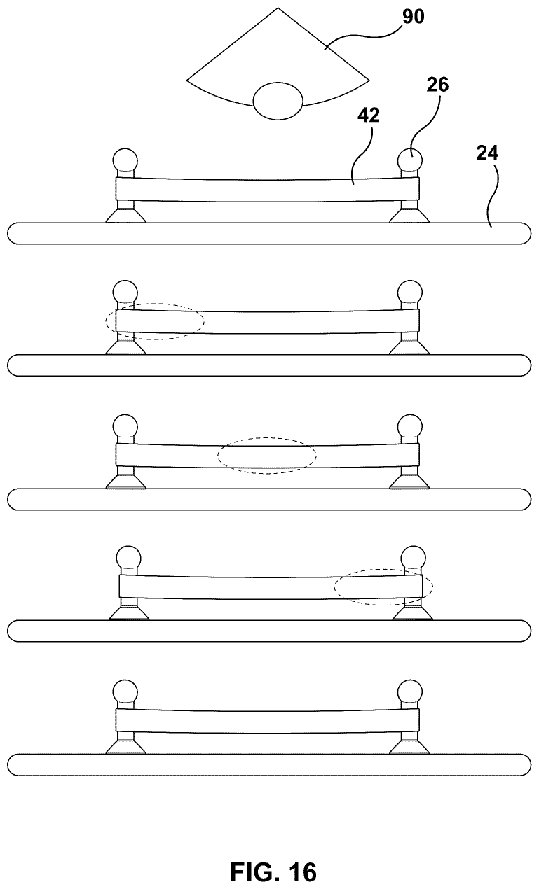

In some embodiments, a vision system 90, preferably capable of high frame rate imaging, is coupled with UV sources and fluorescent light excitation and emission filters. By making cells fluorescent, for example through the use of calcium sensitive dye Fluo-4 AM, electrical activity of cells as well as electrical signal propagation can be measured and quantified (see FIG. 16).

In some embodiments, a construct around the anchors is removed from the system and used directly for implantation or assembled with other sheets or similarly formed tissues and then implanted. Optionally, the construct may be decellularized before implantation. Optionally, a construct may be decellularized and re-cellularized with a different cell type before implantation.

In some embodiments a construct around the pillars is removed from the system and used for cultivated meat applications for human or animal consumption. The construct is made with appropriate cells from relevant animals.

In some embodiments, anchors 26 are fabricated separately and attached to a membrane 24 or other surface that can be used for making cell sheets. Other surfaces may include, for example, responsive (e.g. pH or temperature responsive) surfaces.

To assist in maintaining the growth of a cell construct, a sealed chamber may be formed around a device 20 or around the anchors 26 of a device 20. The sealed chamber may be connected to one or more other devices 20, to cell supporting equipment, to sensors and/or to a control system.

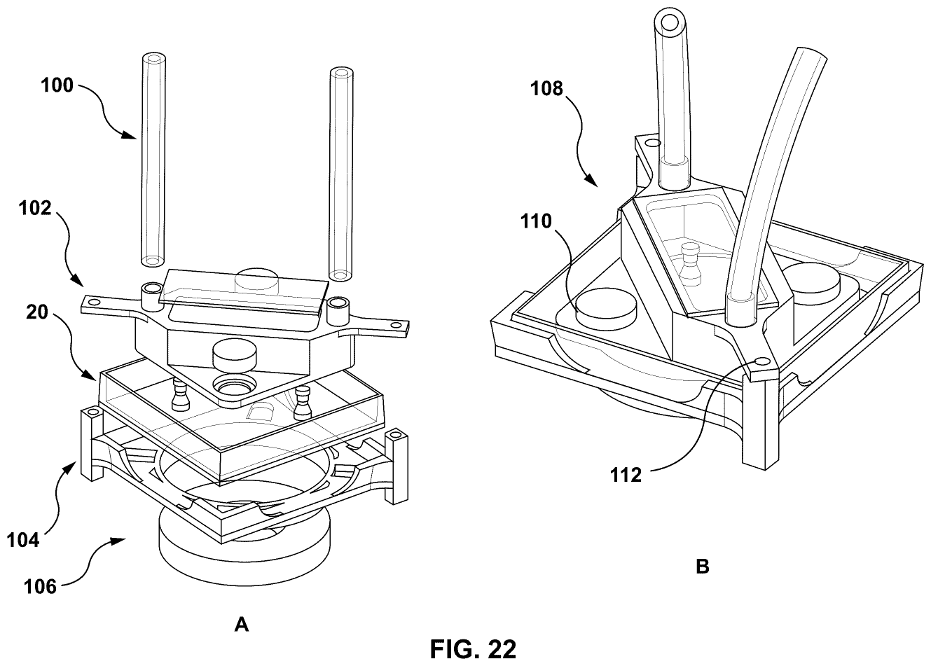

Referring to FIG. 22, a sealed chamber is provided by a system of components (“add-ons”) that may be added onto a device 20. In the examples shown, plastic components were 3D printed with polyethylene terephthalate glycol (PETG) filaments. The device 20 is held in a base 104. The base 104 is configured to hold a bottom magnet 106. A cover 102 provides a chamber covering the anchors 26. The cover 102 is sealed to the upper surface 22 of the device 20. In the example shown, the cover 102 is inserted into the membrane 24 (i.e. between the walls 28 of the membrane 24) and held in place by top magnets 110 and screws 112. The screws 112 connect the cover 102 to the base 104. The top magnets 110 pull the cover 102 towards the bottom magnet 106 to further seal the cover 102 to the membrane 24 to minimize or avoid leaking. Optionally, the bottom magnet 106 is annular with a hole in the center to allow monitoring or microscopy. Similarly, the top side of the cover 102 may have a glass plate 108 to permit monitoring or microscopy. Optionally, the cover 102 or membrane 24 may be adapted to support one or more sensors. Tubing 100 can be used to connect the cover 102 to other equipment, for example a supply of media the cover 102 of another device 20. A pump, for example a peristaltic pump, may be connected to tubing 100 to provide a flow of media.

The size and shape of the cover 102 can be changed to properly fit the tissue or construct that is hanging between the anchors 26. For example, a four-anchor system that supports a hanging sheet would have a larger cover 102 than the one shown in FIG. 22. Optionally sensors, for example oxygen or glucose sensors, may be added to the cover 102 or associated with the cover 102. Optionally, sensors may be held in place by way of one or more of the top magnets 110.

Models representing tissue-tissue interfaces, or methods of growing constructs in communication with other tissues, are enabled by connecting multiple cell culture devices 20 together. Interconnected multi-tissue systems can effectively replicate complex in vivo conditions.

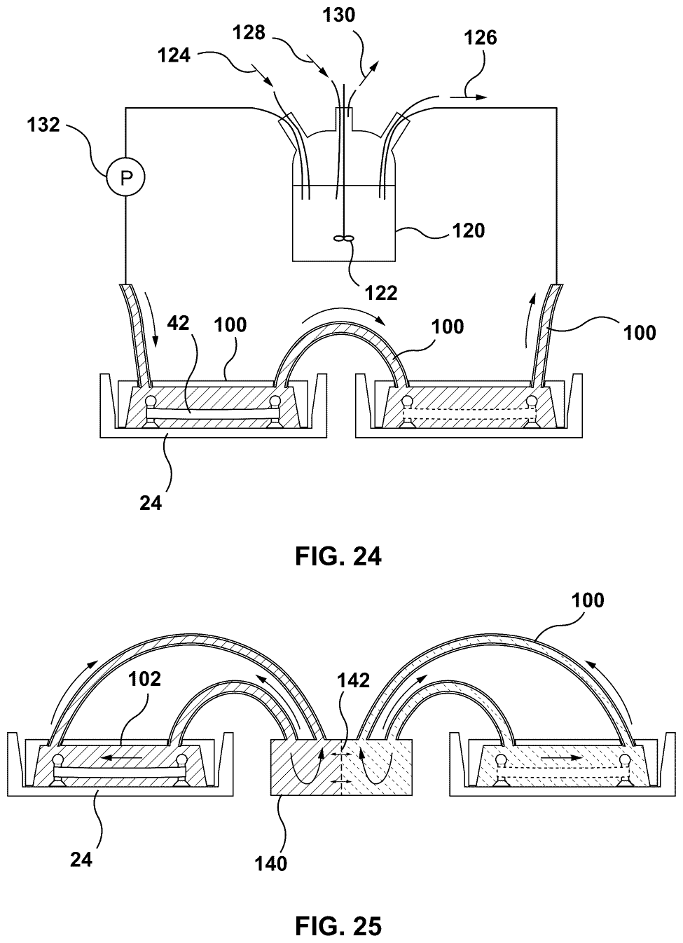

Referring to FIGS. 24 and 25, a physiologically relevant in vitro modeling system may include multiple tissues or constructs and facilitate their cross-talk. In order to recreate this level of complexity, individual models formed using the anchored cell constructs may be connected with each other. In the example of FIG. 24, multiple tissues are connected together directly. In the example of FIG. 25, multiple tissues are connected together indirectly via a permeable membrane. The permeable membrane has a selected molecular weight cut-off and selectively permits molecular transfer between the multiple tissues.

Multiple tissues or constructs, each with their own anchors 26 and support system (e.g. cover 102), can be connected to each other to create a more in vivo like system that connects these constructs to each other, optionally creating an organ-like or body-like system. The constructs can be connected directly to each other, for example where the connected constructs share the same media as in FIG. 24. In the example shown, two covers 102 are connected to each other with tubing 100. The covers 102 are further connected to a spinner flask 120 which operates as a media reservoir. A mixer 122 homogenizes material in the spinner flask 120. New media 124 may be added to the spinner flask and used media 126 may be removed. One or more input gasses 128 may be added to the media in the spinner flask 120, for example by bubbling or by way of a gas transfer membrane. Exhaust gasses 130 may be vented from the spinner flask 120. A pump 132 provides a flow of media from the spinner flask 120 to the first cover 102 to the second cover 102 and back to the spinner flask 120.

Alternatively, multiple tissues or constructs, each with their own anchors 26 and support system (e.g. cover 102), can be connected indirectly, for example where different constructs require different media types and/or mixing is not desirable. As shown in FIG. 25, this can be achieved by circulating the media from different tissues to a chamber 140 with multiple compartments that are separated with each other by a permeable membrane 142 with defined pore sizes and molecular transfer cut-offs. This will allow transfer of certain size molecules, for example different signaling factors or growth factors, between the conditioned media of different constructs to recreate the paracrine effect and the indirect cross-talk between the tissues.

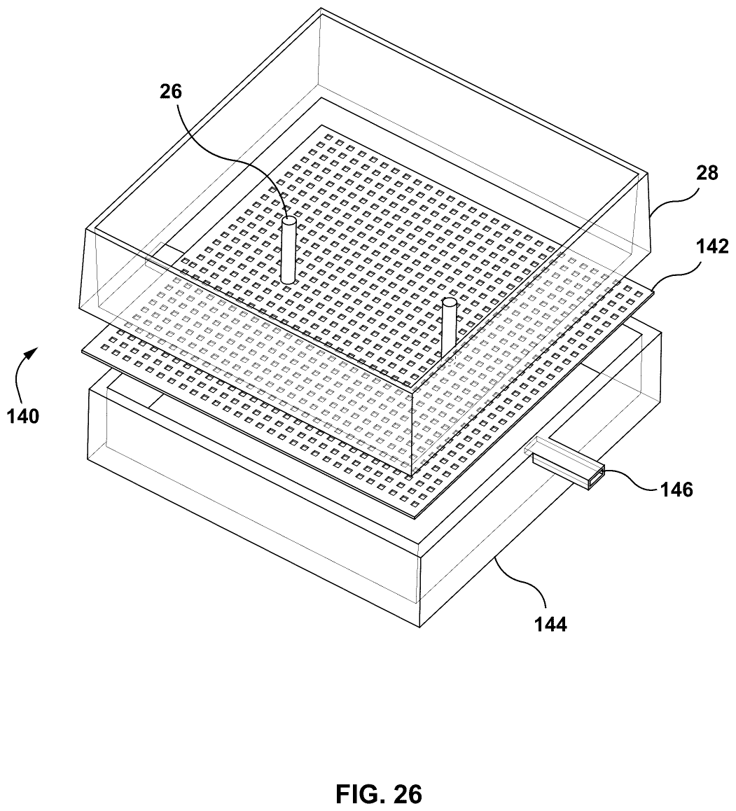

FIG. 26 shows a porous device 140. The porous device 20 has walls 28 and pillars 26 like the device 20 of FIG. 2. However, the bottom of the solid membrane 24 shown in FIG. 2 is replaced with a porous floor 142. The porous floor 142 may be made of, or contain, a semi-permeable membrane or a porous mesh. The porous floor 142 has openings that are small enough to prevent cells from moving from one side of the porous floor 142 to the other. The porous floor 142 can be made from PDMS or other polymers suitable for cell adhesion. Treatment with tannic acid or other methods, for example treatment with ECM, can be performed to make the porous floor 142 more favorable towards cells. Optionally, a cover 102 as shown in FIG. 22 can be placed on top of the porous floor 142. The cover 102 can be modified to seal against the walls 28.

One cell type can be grown on the top side of the porous floor 142 and eventually scraped to form anchored structures. A layer, or optionally multiple layers, of another cell type can be grown on the bottom side of the porous floor 142. This layer can completely cover the porous surface at the bottom side of the porous floor 142. Endothelial or epithelial cells can be used for this layer. A well 144 is placed under the porous floor 142 to contain fluids against the bottom of the porous floor 142. The well 142 optionally has one or more ports 146. The ports 146 may be used, for example, for adding fresh media to a layer of cells growing on the bottom of the porous floor 142, or for connecting multiple culture devices 20, 140 together.

The porous floor 142 can have a responsive surface, for example a temperature sensitive responsive surface, on one side or on both sides. If the porous floor 142 is responsive on both sides it will release the cell layers grown on both sides.

Cell culture can be started with the cell layer on the bottom side of the porous floor 142. The porous device 140 can be flipped over into an inverted orientation to allow the cells to adhere to the bottom floor 142. The porous device 140 is then flipped over again to add more cells on the top side of the porous floor 142.

The methods and devices described herein may be used to produce a multi-organ bioreactor or a self-sustained biological system. In some examples, a bioreactor or system is produced analogous to a bioreactor or system described in U.S. 63/583,488, Integrated Multi-Organ Microphysiological Systems, Dynamic Three Dimensional Organ-in-a-Chamber, filed on Sep. 18, 2023, which is incorporated by reference herein.

In some examples, devices for example as shown in FIG. 24, 25 or 26 may be used to grow multiple cell types in communication with each other. Serum and animal derived components such as fetal bovine serum (FBS) are currently one of the major elements in the complete culture media that is being used for the proliferation and differentiation of mammalian, bovine, porcine, avian, piscine, crustacean, and ovine cells. These components are costly, have batch to batch variability, and can introduce pathogens into the system. For applications such as regenerative medicine, tissue engineering, and cultivated meat, chemically defined and animal component-free media formulations are needed. Currently, recombinant versions of different animal or human growth factor and other components are being used for these purposes but they are expensive. In addition to that, the fermentation capacity for mass producing all of these elements does not exist, and, in some cases, recombinant production of certain elements is not possible.

The devices described herein can be used to produce these required factors or other elements of a cell culture medium. Avatars of different tissues of interest can be grown in one or more parts of a system containing the device. These avatars can be maintained for long culture times, and, in this time, they can be maintained by using small volumes of recombinantly produced growth factors. In return, they produce higher volumes of different growth factors and secrete them for collection or transfer to other parts of the device. Examples of tissues that can be cultured are pancreatic tissue to produce insulin and hepatic tissue to produce albumin and hepatocyte growth factor (HGF) among other elements. By including the proper tissues and organs, a proper formulation of media can be prepared that can be used for different applications.

Different cell types including pluripotent stem cells and iPSCs, as well as adult stem cells, can be used to recreate functioning and mature tissue avatars. For regenerative medicine, human cells may be used, for example cells from the patient or allogenic cells. For cultivated meat, different species' cells can be used to create physiologically relevant tissue avatars. For example, bovine and porcine stem cells can be used to produce a proper growth factor (GF) mix for growing beef and pork respectively.

Optionally, a filtration system can be implemented to remove undesired components of the cellular metabolism, such as lactic acid and ammonia, from this mix before using it for other applications with other cell types. Optionally, the filtration system may be as described in U.S. Ser. No. 17/952,355 filed on Sep. 26, 2022 and/or PCT/CA2023/051264 filed on Sep. 25, 2023, which are incorporated herein by reference.

By increasing the size of each device or using multiple devices, the elements produced in the process can be extrapolated to large scale production without relying on an exogenous recombinant production processes. A device or part of a device producing one or more media elements can either be connected to another device or part of a device where cell proliferation or differentiation is occurring, or a standalone system can be created wherein liquid can be transferred between this media element production system and any standalone bioreactor.

In some embodiments, innate and adaptive immune cells are incorporated in one or more parts of a device for GF and cytokine production. Species differences can be considered for preparing a proper mixture of growth and signaling factors.

In some embodiments, a device is used as a first part of an open-loop system where GFs produced in the device are collected and transferred to a second container where cells are grown and differentiated for other regenerative medicine or cultivated meat applications. It is also possible to create a closed-loop system where the conditioned media from a second container is fed to a device as a feedback loop. Tissues and organs in the device will understand the concentration of different elements and will adjust their behavior accordingly. For example, pancreatic cells can act as sensors for glucose in this closed-loop system and adjust their insulin production levels. Similarly, hepatocytes can act as albumin sensors.

In some embodiments, a different environment is created for some cells in the device as compared to another part of a device used for growing the cells. For example, by lowering the temperature or creating a hypoxic environment, production of certain GFs can be induced while other cell types proliferation can be optimized in their own vessels. Different devices containing different tissues and organs with different distribution patterns and environmental conditions can be used in parallel, independent from each other, or in series, and their output can be delivered to the final growth vessel. Feedback systems can be also established between the devices themselves.

In some embodiments, a cell sheet is produced as an intermediary or precursor product as part of a method of producing a 3D cell construct. The devices and methods described herein may also be used to produce cell sheets (e.g. cell sheets analogous to the cell sheets produced as intermediary products) as final products.

In some embodiments, a cell culture device is made of multiple materials, for example multiple elastomeric materials. In some examples, PBAT and a silicone resin such as PDMS are combined. PBAT and PDMS do not typically bind to each other. However, integral PDMS and PBAT castings can be made by transitioning from PBAT to PDMS through an intermediate material made from a mixture of PBAT and PDMS. In some examples, the device includes a membrane made of a non-elastomeric material and anchors made of an elastomeric material.

Anchors may be added to other substrates suitable for cell culture and cell sheet formation. For example, anchors may be added to a membrane with an environmentally responsive (e.g. pH or temperature responsive) surfaces. In some embodiments, anchors are adhered to the cell growing surface of a membrane. In other embodiments, a membrane is molded onto or around anchors. In other embodiments, anchors are threaded or press fit into openings in the membrane. In other embodiments, anchors are inserted through holes passing through the membrane.

Optionally, anchors may be placed near cell layers, touching cell layers, or partially embedded in cell layers, without protruding entirely through cell layers. The anchors may be put in place before, during or after growing the cell layers. For example, anchors may be lowered from above cell layers to be very near the surface of the cell layers, or to touch the top of the cell layers without unduly pressing the cell layers. This can be done, for example, at any point between when the first layer of cells is grown on a substrate to when removal of the cell layers from the substrate begins. Alternatively, anchors may be extended from above a substrate to near the surface of a substrate, for example to be spaced from the substrate by a distance less than, equal to, or slightly greater than the expected thickness of the cell layers, before beginning cell culture. In some cases, it may be possible to detach a cell sheet and drape it over a set of pillars that were not previously associated with the cell sheet. However, while these methods may have some advantages in some applications, they are not expected to be as reliable in the formation of remodeledled cell constructs as a method wherein the cell layers are grown around anchors already extending from the surface of the substrate (whether by being integral with, attached to or simply touching the surface of the substrate) such that the anchors form and extend through openings in the cell layers.

Optionally, a form may be provided between two or more anchors. A cell sheet that detaches from the membrane of a cell culture device encounters the form as the cell sheet becomes located on the anchors. The cell sheet remodels in contact with the form. The form may thereby influence the shape or one or more surfaces of a cell construct. The form may be, for example, in the shape of a rod, a tube, or the form may have a flat surface. The cell construct may remodel against one side of the form or the cell construct may surround the form. In some examples, the form is cylindrical or tubular. Depending on factors such as the thickness and height of the pillars, a cell sheet can surround the form to create a tubular construct or a cell sheet can cover only the bottom of the form to create a partially tubular construct. Such constructs can be used in applications such as blood vessel formation or as vascular grafts.

### Example 1: Formation of Hanging Muscle Fibers Using Rabbit Myoblast Cell Sheets as Building Block Using Patterned PDMS Membranes Made Ecoflex 00-30 Pillars

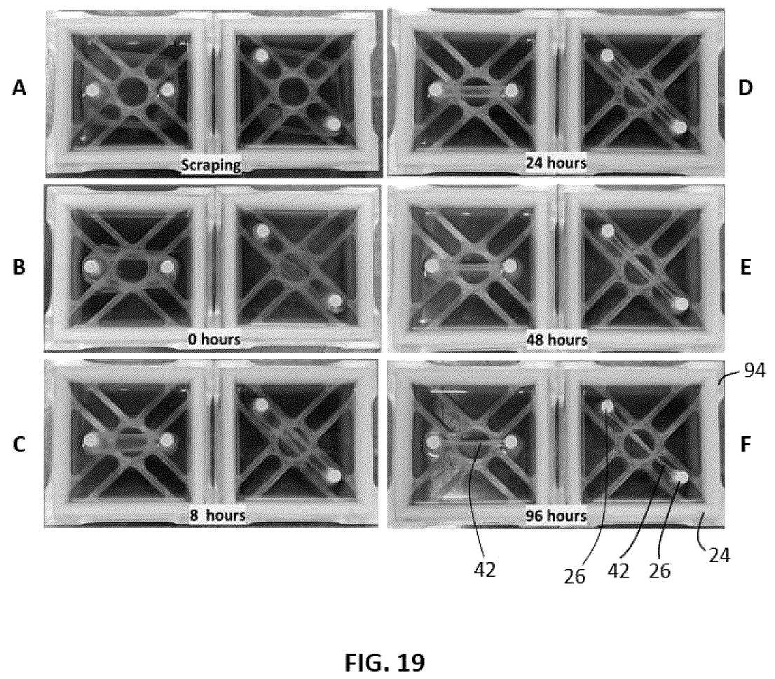

In this experiment, rabbit myoblasts are differentiated to skeletal muscle cells and used to form a cell sheet with aligned cells. These cell sheets are induced to form a fiber hanging between two pillars as shown in FIG. 19 by way of the following steps:
1. A master mold is 3D printed using Original Prusa i3 MK3S+ and ABS filament. Nozzle size is 0.4 mm and printing speed is 60 mm/s.
2. Ecoflex 00-30 (a 1:1 volume ratio of parts A and B are mixed) is used to fill the pillar cavity in the mold up to the conical base. After 20 minutes that Ecoflex is half cured, a 1:1 volume ratio mixes of Exoflex and PDMS SYLGARD 184 (PDMS solution is made with a 10:1 ratio of the base and curing agent) is used to fill the conical base. After 2 more hours that this mix is partially cured, the rest of the mold is covered with PDMS and is allowed to completely cure at 60° C. for 4 hours. Eventually the whole device is pulled off the mold with care not to tear the pillars from the base membrane.
3. After sterilizing the device with 70% ethanol, it is treated with 3 mL of sterile aqueous solution of tannic acid with a concentration of 50 mg/mL for 72 hours, after which it is washed three times with deionized water to eliminate any traces of tannic acid.
4. Rabbit myoblasts (Sigma-Aldrich, RB150-05) were grown up to 80% confluence in 10 cm petri dishes in their growth medium (Sigma-Aldrich, RB151-500). They were dissociated using trypsin and 5×10⁵ of them were added to the membrane in the same growth medium. This is considered day 1 of the sheet formation process and the number of cells will result in an >80% confluency once cells adhere to the membrane. Cells also cover the conical base of the pillars as well but not the vertical shaft of the pillars.
5. On day 3 of the experiment, medium is switched to the differentiation medium of the cells (Sigma-Aldrich, 151 D-250) plus enough L-ascorbate-2-phosphate to achieve a concentration of 100 μg/mL. This media is refreshed every two days till the end of the experiment.
6. On days 4, 6, and 8, the same number of cells are added on top of the membrane directly to the differentiation medium. The newly added cells adhere on top of the previous layers of cells and differentiate to skeletal muscle cells.
7. On day 18, the cells and their ECM are scraped off using a cell scraper (Sigma-Aldrich, CLS3010-100EA) to the base of the pillars. At this point, since cells have lost their anchorage on the membrane, they start to remodel their cytoskeletons and show shrinkage of the partially scraped sheets. In some trials, cells between the pillars detach spontaneously from the membrane after a period of time. In other trials, after a waiting period of about 1-3 hours, cells are scraped from the membrane between the pillars. Eventually, after about 24-48 hours, the cells form a fiber like structures while hanging between the pillars and using them as anchors. The cells slowly move the fiber up towards the middle of the pillar shaft.

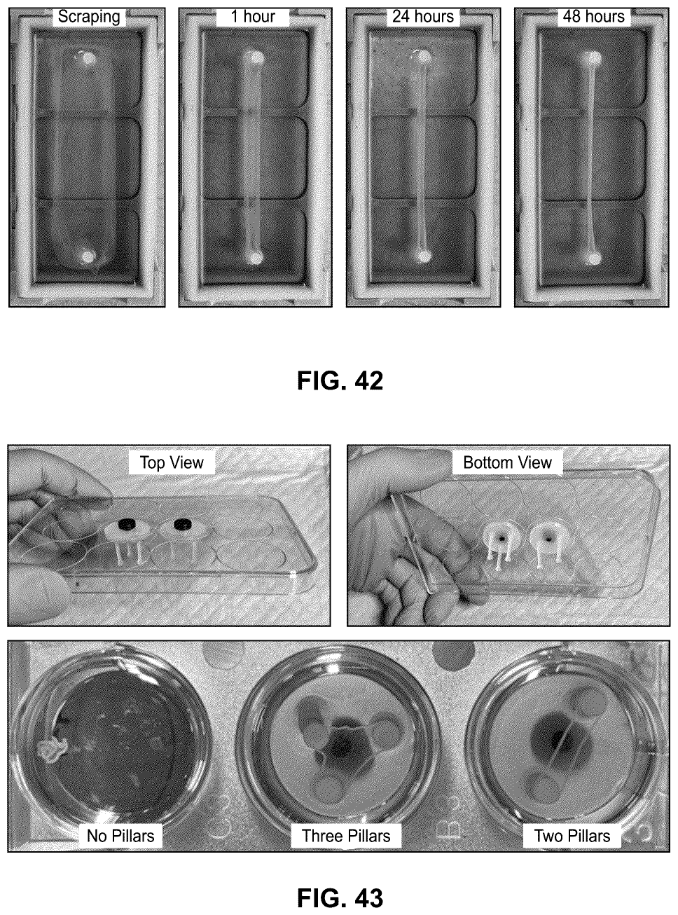

The membrane in FIG. 19 is approximately 30 mm by 30 mm in plan view. In a similar example, a longer muscle fiber (shown in FIG. 42) is produced on another membrane that is longer than the membrane used in FIG. 19. The size of the 3D tissue constructs can be manipulated by controlling the surface area and dimensions of the culture device. In this instance, a longer muscle fiber was fabricated using rabbit skeletal muscle cells (SKMCs) by extending the length of the culture device from 3 cm to 7 cm, which resulted in a muscle fiber that was 5 cm in length. As the width of the membrane device remained constant, the thickness of the muscle fiber stayed the same.

### Example 2: Formation of Hanging Sheets Using Rabbit Myoblasts on a 4-Pillar System

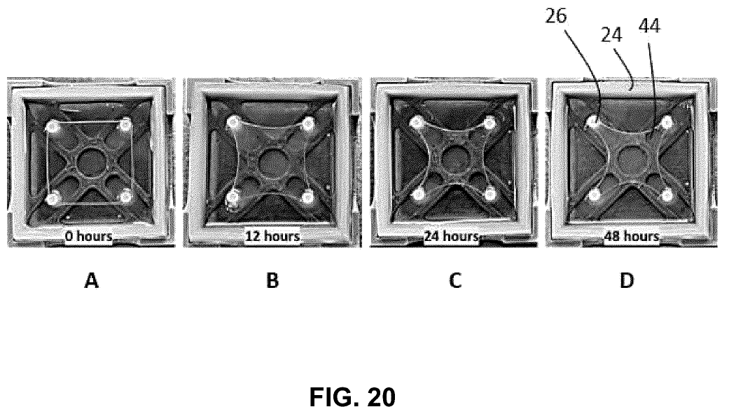

In this experiment, rabbit myoblasts are differentiated to skeletal muscle cells and used to form a cell sheet with aligned cells. These cell sheets are induced to form a hanging sheet between four pillars as shown in FIG. 20 by way of the following steps:
1. A mold is 3D printed similar to the mold in example 1 but instead of two pillars, four pillars are included close to each vertex of the square membrane. These pillars are about 3 mm away from each edge. The membrane as a whole is about 30 mm by 30 mm in plan view.
2. Steps 2-6 of example 1 are repeated here.
3. On day 18, the cells and their ECM are scraped off using a cell scraper (Sigma-Aldrich, CLS3010-100EA) to the base of the pillars from the walls of the membrane.
4. The partially scraped sheet shows some shrinkage after 6-8 hours and forms a stronger construct that exerts contractile force on the rest of the cells that are still attached to the membrane. The attachment of the rest of the cells to the membrane is weakened due to this contractile force. At this point, instead of using a scraper a pipette tip is used to perform the rest of the scraping process. The rest of the cells are induced to completely lift off of the membrane by using a pipette tip and pushing it in between the sheets and the membrane with a 30-45 angle. Force is applied gently to slowly detach the sheet from the membrane and not tear it apart.
5. After the whole sheet is lifted off of the membrane, cells continue remolding and shrink the sheet over the next 24-48 hours. This slowly moves the sheet up towards the middle of the pillar shaft.

### Example 3: Decellularizing Hanging Sheets and Using them as an ECM for Growing Other Cell Types

In this experiment, a sheet hanging between 4 pillars, similar to the sheet made in example 2, is decellularized and its ECM is used to grow other cell types as shown in FIG. 21 by way of the following steps:
1. 48 hours after the hanging sheets are formed using rabbit myoblasts differentiated to skeletal muscle cells, the hanging sheets are decellularized. First media is removed and sheets are washed in PBS to remove any residual media. Then a 0.2% v/v solution of Triton X-100 in deionized water is added. The sheets are incubated with the detergent solution at 4° C. for 48 hours. Eventually sheets are washed with PBS, 4 times, to remove the detergent molecules and then with 70% ethanol to sterilize them and then again with sterile PBS to remove the ethanol.
2. At this point, only the ECM components of the sheet remains. New cells, in this case a lung A549 cell line, are added in a small volume of the media on top of the decellularized sheet. A low volume of media is used to make sure the cells are not spilling over to the bottom of the membrane. This allows cells to adhere to the ECM and not to the surface of the membrane.
3. After incubating these cells at room temperature for 30 minutes, they form strong enough bonds with the ECM. At this point more media is added to cover the bottom of the membrane and submerge the cells that are on top of the ECM that is hanging between the pillars and away from the bottom of the membrane.
4. With continued incubation, cells continue to proliferate and perform their natural functions on the decellularized sheet.

### Example 4: Connecting Multiple Tissues to Each Other Using the Modular System

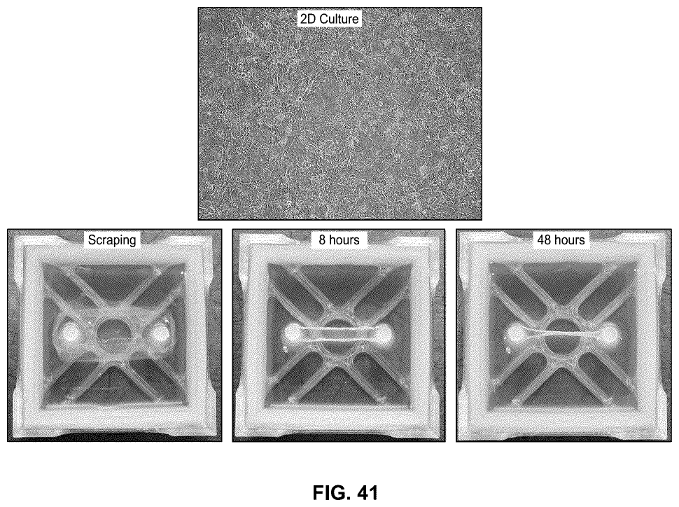

In this experiment, 3D cell constructs are formed for both skeletal muscle and another cell type, in this example hepatocytes. Each of these constructs are made on their own pillar system and they are then connected to each other by way of a system similar to the system of FIG. 24. The two constructs create their cross-talk, similar to in vivo conditions. The experiment includes the following steps:
1. Muscle fibers are formed in a manner similar to Example 1.
2. For making a hepatocyte 3D construct, human induced pluripotent cells (iPSCs) were differentiated into hepatocytes on minimally patterned membranes. The hepatocyte monolayer culture is then used to form a hepatocyte cell sheet which is subsequently transformed into a 3D anchored tissue construct. Human IPSCs from Stem Cell Technologies (iPSCdirect™ Healthy Control Human iPSC Line, Stem cell Technologies Catalog #SCTi003-A) were used. They were grown in their growth media (mTeSR plus, Stem cell Technologies Catalog #100-0276) until 70% confluent.
3. These cells were then passaged as single cells using dissociation agent ReLeSR™ (Stem cell Technologies Catalog #100-0483). These single cells were transferred to a minimally patterned membrane (i.e. a membrane made on a 3D printed mold that was solvent vapor treated to reduce depth and roughness of the membrane patterns) to achieve 90% confluency the next day. The cells were passaged in their growth medium supplemented with 10 μM Y-27632 (Stem cell Technologies Catalog #72302).
4. Cells were differentiated to hepatocytes beginning on day 2 using the kit STEMdiff™ Hepatocyte Kit (Stem cell Technologies Catalog #100-0520). Differentiation took 20 days and requires stepwise treatment with 4 different media types according to the protocol provided by the vendor.
5. On day 21 cells had proper hepatocyte morphologies (as shown in FIG. 41) and were ready to be scraped to form a 3D hepatocyte construct. The scraping procedure was similar to what was performed for muscle fibers and a 3D hepatocyte construct was formed after 48 hours (as shown in FIG. 41).
6. At this point, add on modules as shown in FIG. 22 for each of the 3D constructs were inserted into the membranes to create a chamber around each construct. A system was configured as in FIG. 24 to create a flow of media between the two constructs by connecting them together through sterile silicone tubing and using a peristaltic pump.

### Example 5: Preparation of Fiber Graft Implant

Culture devices were created using a combination of Ecoflex™ 00-30 and polydimethylsiloxane (PDMS) resins cast on 3D-printed master molds. Various master mold designs with different pillar configurations were developed, each with a 30×30 mm base and 10 mm high walls. The pillars, 8 mm in height, featured a conical base and cylindrical head with a 1.5 mm shaft diameter. CAD models of the molds were converted to G-codes using PrusaSlicer 2.4.2 and printed with acrylonitrile butadiene styrene (ABS) filament on an Original Prusa i3 MK3S+3D printer. After preparing the master molds, the pillar heads and shafts were filled with Ecoflex™ 00-30 resin, created by mixing equal volumes of parts A and B and degassing. A syringe and gauge 20 blunt needles were used for injection. A 30-minute partial setting period at room temperature was allowed before filling the pillar's conical part with a 1:1 volume mix of Ecoflex™ and PDMS. A 10:1 volume ratio of Sylgard 184 and its curing agent was maintained for the PDMS mix used in the device's walls. PDMS was also used to cover the master mold's base. After 24 hours, all components of the culture devices were fully cured and fused, and the devices were removed from the molds. Pillars were arranged either diagonally, with centers 9 mm from the device corners, or in a line forming a perpendicular bisector of the membrane wall, with centers positioned 6 mm from the device edges. The PDMS surfaces of the culture devices were treated with a 50% aqueous solution of tannic acid (TA) (Sigma-Aldrich, 403040) for 72 hours to optimize hydrophilicity for cell attachment.

After treating the culture devices with TA solution, they were washed three times with deionized water and autoclaved in sterilization bags. Each device was placed on a 3D printed support to create a gap between the device and the petri dish, improving oxygen transfer. The supports were 3D printed using polyethylene terephthalate glycol (PETG) and sterilized with 70% ethanol. On the first day of the cell sheet engineering process, culture was initiated with 5×105 skeletal muscle cells (SKMCs) in 3 mL of growth medium to reach 80% confluency by the next day. On the third day, when cells were nearly 100% confluent, the medium was switched to differentiation medium. This differentiation medium was refreshed every other day until day 18, with additional 5×10⁵ SKMCs introduced to each device on days 4, 7, and 10. By day 18, the multi-layered cell and extracellular matrix (ECM) growth on the membrane were ready to be delaminated as a sheet. A sterile scraper with a 16 mm blade was gently used to scrape the cell layer from the device edges, detaching the cells from the walls and bottom membrane. This procedure was performed for all four edges of the device. A simple shaking action was then sufficient to detach the entire cell sheet from the membrane. When using culture devices with pillars, cell sheets were created by partially scraping the cell layer from the edges to the bases of the pillars, followed by a 6-hour incubation period. A 1 mL pipette tip was then used to detach the remaining cell layer from the membrane, forming the hanging fibers between pillars.

After 4 days that the hanging fibers were formed, they were decellularized using a detergent solution containing 0.2% Triton X-100 and 0.2% Tween-20 in PBS. The medium was discarded, and the fibers were washed twice with sterile PBS to remove any residues. The detergent solution was added, and the fibers were incubated at 4° C. for 48 hours, with a refresh at the midpoint. The decellularized fibers were washed twice with sterile PBS, soaked in 70% ethanol for 5 minutes at room temperature for sterilization, and then washed with PBS again to remove ethanol residues.

The decellularized fibers may be used as fiber graft implants to assist with the repair of volumetric muscle loss in humans or other animals. Grafts may be added to an area of exposed muscle. Grafts are measured and cut to match the muscle deficit. The grafts may be were sutured directly onto the surrounding muscle tissue, for example using one of three potential suture types: suture 9-0, prolene suture 6-0, or monofilament absorbable sutures Polyglecaprone 25. The skin and fascia may be subsequently closed.

The culture device and methods described herein may be used to produce other structures, with or without cells, for implantation in vivo for regenerative medicine or tissue engineering applications. Optionally, the cells may be derived from a patient who will receive the graft or implant. For example, the acellular graft described above may be alternatively used for tendon or ligament repair.

### Example 6. Materials and Methods

Shahin-Shamsabadi A. et al., Anchored Cell Sheet Engineering: A Novel Scaffold-Free Platform for in vitro Modeling (Adv. Funct. Mater. 13/2024), including its supporting information (including supplemental figures, tables and videos) is incorporated herein by reference.

### 6.1 Culture Device Fabrication

Culture devices comprising multiple materials were manufactured using Ecoflex™ 00-30 and Polydimethylsiloxane (PDMS) resins casted on 3D-printed master molds. CAD models of various master mold designs, incorporating different numbers and distributions of pillars, were conceived. The designs encompassed a 30×30 mm base for cell culture surface area and walls with a height of 10 mm. Pillars, standing at 8 mm high, had a conical base and a cylindrical head, with a shaft diameter of 1.5 mm. Master molds were 3D printed using acrylonitrile butadiene styrene (ABS) filament on an Original Prusa i3 MK3S+3D printer equipped with a 0.4 mm nozzle.

Following 3D printing, half of the master molds underwent acetone vapor treatment for 30 minutes at room temperature, intended to mitigate 3D printing patterns on the part's surface. Each 3D printed part was placed in a 5×5×5 cm plastic box with a fitted lid. A Kimtech wipe, saturated with 2 mL of acetone, was attached to the lid of the box, ensuring the wipe was fully soaked but without any dripping. After a 30-minute treatment, the master molds were removed from the boxes and left in room condition for 24 hours, allowing the absorbed acetone to evaporate.

Upon preparation of the master molds, resin casting step was executed. For pillar molds, pillar heads and shafts were filled with Ecoflex™ 00-30 resin. The resin was prepared by mixing equal volumes of parts A and B and degassing using a vacuum desiccator. The blend was then injected into the pillars with a syringe and gauge 20 blunt needles (McMasterCarr). The resin was allowed a 30-minute partial setting period at room temperature before the pillar's conical part was filled with a 1:1 volume mix of Ecoflex™ and PDMS. The PDMS mix maintained a 10:1 volume ratio of Sylgard 184 and its curing agent (Dow corning). The same blend was utilized for the device's walls. After an additional 6-hour room temperature incubation, a PDMS-only mix was used to cover the master mold's base. After 24 hours, all parts of the culture devices were cured and fused, and the culture device was peeled off the master mold. For fabricating culture devices without pillars, the process was similarly conducted except for using the Ecoflex™-PDMS blend solely for the walls. The membranes produced from the original 3D printed master molds were referred to as “Patterned Membranes”, whereas those made with acetone-treated master molds were termed “minimally patterned membranes”. For the 2-pillar system, the pillars were arranged in one of two ways: either diagonally, with the pillar centers positioned 9 mm from the device corners, or in line, thereby forming a perpendicular bisector of the membrane wall, with centers situated 6 mm from the device edges.

### 6.2. Treatment of Devices for Cell Culture

The PDMS surface of the culture devices was treated with an aqueous solution of tannic acid (Sigma-Aldrich product #403040) to modulate its hydrophilicity for optimal cell attachment. Surfaces underwent treatment with 25, 50, or 100 mg/mL tannic acid solutions in deionized water, for durations of 24 or 72 hours. Contact angle measurements were performed by imaging 10 μL droplets on each membrane, utilizing a portable digital microscope with 50-1000× magnification and analyzing the images using the Contact Angle Plugin in ImageJ. In each instance, four PDMS membrane pieces were tested and average±mean values were reported.

Scanning Electron Microscopy (SEM) was conducted using the Axia ChemiSEM in High Vacuum (HiVac) mode and ETD detector (secondary electron mode) with a 3.0 nm spot size at 30 kV for both patterned and minimally patterned PDMS membranes to image surface patterns of the membranes. The impact of tannic acid treatment on PDMS surface composition was analyzed by Energy-dispersive X-ray spectroscopy (EDS).

### 6.3. Cell Culture Process

Primary rabbit skeletal muscle (SKMC) myoblasts (Sigma-Aldrich, product #RB150-05) alongside the lung A549 epithelial cell line (ATCC product #CCL-185) were used in this study. SKMC were grown in growth medium (Sigma-Aldrich, product #RB151-500) until they reached 70% confluency, prior to passaging and further utilization. Their differentiation was induced using differentiation medium (Sigma-Aldrich, product #151D-250) supplemented with 100 μg/mL 2-Phospho-L-ascorbic acid (Sigma-Aldrich, product #49752). A549 cells were cultivated in Ham's F-12K (Kaighn's) Medium (Gibco™, product #21127022) containing 10% Fetal Bovine Serum (FBS) (Gibco™, qualified, Canada, product #12483020).

### 6.4. Cell Sheet Engineering

For cell sheet engineering applications, culture devices treated with 50 mg/mL tannic acid (TA) solution for 72 hours were used. After TA treatment, the culture devices were washed three times with deionized water and subsequently autoclaved in sterilization bags. Each device was then secured atop a 3D printed support to create a gap between the device bottom and the petri dish in which they were housed, thereby improving oxygen transfer from beneath. The supports were printed with polyethylene terephthalate glycol (PETG) and sterilized with 70% ethanol.

On the first day of cell sheet engineering process, culture was initiated with 5×10⁵ SKMCs in 3 mL of growth medium to achieve 80% confluency by the next day. On the third day, with cells at 100% confluency, medium was switched to differentiation medium. This differentiation medium was refreshed every other day until day 18, while additional 5×10⁵ SKMCs were introduced to each device on days 4, 7, and 10. By day 18, the multi-layered cell and ECM growth on the membrane were ready to be delaminated from the membrane as a sheet. To achieve this, a sterile scraper with a 16 mm blade (Sigma-Aldrich, product number #C6106) was employed to gently scrape the cell layer from the device edges, detaching the cells from the walls and bottom membrane of the culture device. This procedure was carried out for the four edges of the device. At this point, a simple shaking action was sufficient to detach the entire loosely attached cell sheet from the membrane.

### 6.5. Anchored Cell Sheet Engineering

The cell culture process was carried out similarly to the previous section when utilizing culture devices with pillars. The cell sheets were created by first partially scraping the cell layer from the edges of the culture device, all the way to the bases of the pillars, followed by a 6-hour incubation period. Subsequently, a 1 mL pipette tip was utilized to detach the remaining cell layer still affixed to the membrane, thereby forming either a fiber suspended between two pillars or a flat sheet hanging between four or six pillars. These hanging cell sheet-based constructs were maintained in culture with differentiation medium, allowing the cells to further remodel their micro-structure.

6.6. Integration of 3D Spheroids with Sheets

The fabrication of master molds for spheroid creation was performed similar to before, with the design incorporating half spheres 5 mm in diameter, positioned on the mold's surface. This arrangement ensured the formation of half-sphere like indentations in the membrane after casting PDMS. Like before, the master mold underwent treatment with acetone vapor to smooth surface features. Following the detachment of the PDMS culture devices from the master mold, these were washed with 70% ethanol and allowed to dry within a biological safety cabinet. Subsequently, they were treated with a 0.1% wt/V solution of Pluronic F127 (Sigma-Aldrich, product number #P2443) in deionized water, followed by multiple washes with sterile PBS to remove any residues.

SKMCs, grown to 70% confluency in their growth medium within a 10 cm petri dish, were treated with fresh growth medium containing a 1:1000 dilution of Nile red (Sigma-Aldrich, product number #19123) stock solution. This stock solution was prepared by dissolving 10 mg of the Nile red powder in 1 mL of acetone. Following a one-hour treatment period, the Nile red-containing medium was discarded, and the cells were washed with PBS before dissociation and addition to the spheroid fabrication culture device. Each device, which contained 13 spots for spheroid formation, was supplemented with a total of 3×10⁶ cells. After a 24-hour incubation period, the spheroids were ready.

The master mold for the fabrication of sheet-spheroid integration culture devices was designed similar to previous iterations, with pillars positioned diagonally and a half-sphere 3 mm in diameter located in the device's center. Sheet formation was conducted similar to the previously outlined steps using SKMCs but with more cells (7.5×10⁵) added on the first day, with one additional step. On the second day, multiple pre-fabricated spheroids were transferred using a sterile 1000 μL pipette tip with a large orifice from their culture device to the indentation in the center of the sheet formation culture device. At this point, the first layer of SKMCs had already reached confluence. An additional 24 hours of incubation was necessary for the spheroids to integrate with the cell layer before adding subsequent layers of cells. The scraping and sheet formation process was completed on the 12th day.

### 6.7. Air Liquid Interface Culture System

The hanging cell sheets in the 4-pillar system were formed, and after 48 hours were decellularized using a detergent solution containing 0.2% V/V of Triton X-100 (Sigma-Aldrich, product number #X100) and 0.2% Tween-20 (Sigma-Aldrich, product number #P9416) in PBS. Initially, the medium was discarded, and the sheets were washed twice with sterile PBS to wash away any medium residues. The detergent solution was then added, and the sheets were incubated at 4° C. for 48 hours with the detergent solution, refreshed at the midpoint of the process. The decellularized sheets were washed twice with sterile PBS to remove the detergent, soaked in 70% ethanol for 5 min at room temperature for sterilization, and then washed with PBS again to remove ethanol residues.

Following this, the hanging ECM sheets were recellularized with A549 cells. Briefly, A549 cells were dissociated using trypsin and then resuspended in their growth medium to achieve a density of 2×10⁶ cells/mL. 0.5 mL of this cell solution was slowly added on top of each hanging ECM sheet to prevent any spill over to the bottom of the culture device. After a 30-minute incubation period at room temperature, which allowed the cells to adhere to the ECM sheet, 3 mL of fresh growth medium was gently added to fully submerge the sheet and cells. These were then transferred to the incubator. After 24 hours, the cells achieved confluency on the ECM sheets. At this point, one group of cells was maintained in a submerged condition, while the second group was transitioned to an air-liquid interface state by lowering the medium volume from 3.5 mL to 2 mL, so that the hanging ECM sheet containing cells floated on the medium, with the top surface containing the cells exposed to air. The medium was refreshed for all samples every other day for an additional 10 days before culture was terminated.

For the next step, cells were washed with PBS and then fixed with a 10% formalin solution (Sigma-Aldrich, product number #47608) in PBS with for 5 minutes. After washing with PBS to remove fixative residues, staining was performed using DAPI (Sigma-Aldrich, product number #D9542) and Phalloidin-Atto 488 (Sigma-Aldrich, product number #49409) following the appropriate protocols. Fluorescent microscopy was performed using a BioTek Lionheart Automated microscope with suitable filters.

### 6.8. Cell Sheet-Derived ECM (CSdECM) as Coating

Cell sheets were formed using the previously described protocol and washed with PBS immediately after scraping and sheet formation. Each sheet was decellularized, and the ECM components were transferred to a microtube containing 0.5 mL of a 3M solution of Urea (Sigma-Aldrich, product number #U5378) in PBS containing 0.1M acetic acid (Sigma-Aldrich, product number #A6283). Sheets were incubated in this solution for 48 hours at 4° C., and solubilization was facilitated by sonication, using 2 cycles of 4 W power for 5 seconds each. The solution was then centrifuged at 15,000 rpm for 15 minutes to precipitate undissolved ECM components. To eliminate DNA residues, 10 μL of DNase I solution and 5 μL of Ca/Mg2+ to activate it were added and, after 10 minutes incubation at room temperature, 5 uL of EDTA solution from the kit (ThermoScientific, product number #EN0521) was added to quench the reaction. The solution was then dialyzed against deionized water at 4° C. for 72 hours, refreshing the deionized water daily. A Pur-A-Lyzer™ Midi Dialysis Kit (Sigma-Aldrich, product number #PURD10005) with a 1 KDa cutoff was used. The solution was then sterile-filtered using a 0.2 μm syringe filter and stored at −20° C. for further use.

Non-treated 6-well plates (Costar, product number #3736) were treated with CSdECM solution. 2 mL of PBS containing a 1:50 dilution of the dialyzed CSdECM solution was added to each well, and incubation was performed at 4° C. overnight. The next day, wells were washed with PBS and 10⁵ of either SKMC or A549 cells were added to each well in their respective growth media. Similar numbers of cells were added to wells of the same well plate or tissue culture treated ones without CSdECM treatment as controls. Imaging was performed using an inverted EVOS cell imaging system with 10× magnifications in phase contrast mode on days 2 and 4 to evaluate cells' attachment, morphology, and growth over time.

### 6.9. Structural Evaluation of Tissue Constructs

Different tissue constructs, such as cell sheets immediately post-scraping, hanging sheets 48 hours post-scraping, and hanging fibers 6 days post-scraping, were fixed in 10% formalin. Following stepwise dehydration in 25, 50, and 75% ethanol in deionized water, samples were embedded in paraffin. 5 μm thick sections were cut from the blocks onto Fisherbrand™ Superfrost™ Plus microscope slides and staining was performed using hematoxylin and eosin (H&E), Movat Pentachrome, Masson's Trichrome, and Picrosirius Red. The slides were scanned with a Leica Aperio AT2 slide scanner at 40× magnification, and the Picrosirius Red stained slides were scanned using the polarizing Abrio imaging system. SEM was carried out as previously described, utilizing Axia ChemiSEM in High Vacuum (HiVac) mode and ETD detector (secondary electron mode) with a 3.0 nm spot size at 30 kV. Both decellularized sheets and anchored fibers were imaged. Decellularization was performed similarly to before and after thoroughly washing the samples with PBS, they were freeze-dried to prepare them for SEM.

The mechanical properties of the anchored fibers, long (5 cm) fibers as well as their decellularized version, and short (2 cm) fibers, formed in the culture devices with diagonally positioned pillars, were evaluated using the UniVert mechanical testing equipment from CellScale. Briefly, fibers were removed from the pillars and mounted on plastic sheets using liquid glue. A metallic rod was pushed through the opening in the fiber previously filled with the pillars. The rods were also glued to prevent the fibers from slipping under tension. The plastic sheet was then mounted on the UniVert and was cut before starting the tensile test. The plastic sheet was utilized to avoid any damage to the fiber while it was being mounted. A 10N loadcell was used and fibers were stretched at a rate equivalent to 50% of their initial length per minute. Fibers were submerged in water during the test to avoid drying. The linear slope of the Force-Displacement graphs was used to compare different samples. Three samples were tested under each condition and the results were reported as average±mean.

### 6.10. Proteomics Analysis of Cell Sheets

Sheets made with SKMC were prepared for proteomic analysis by lysing in lysis buffer containing 0.5 M Tris pH 8, 2% SDS, 1% NP-40, 1% Triton X-100, 5 mM EDTA, 50 mM NaCl, 10 mM Tris(2-carboxyethyl)phosphine, and 40 mM Chloroacetamide. The lysis process was facilitated by sonication, using 2 cycles of 4 W power for 15 seconds each. Post sonication, the lysate samples were heated at 95° C. for 20 minutes for protein denaturation. The proteins in the lysate were precipitated using a chloroform/methanol solution. The precipitated proteins were then suspended in 50 mM Triethylammonium bicarbonate and was digested with trypsin (Pierce, 4 μg per sample, 1:50-1:100, at 37° C. overnight). Post digestion, the samples were desalted using a Millipore C18 ZipTip and lyophilized using a speedvac. The desalted samples were resuspended in a solution of 2% acetonitrile and 0.1% formic acid for LC-MS/MS analysis. The peptide samples were analyzed using a ThermoScientific Exploris 480 mass spectrometer coupled with an EASY-nLC 1000 nano-LC system. The samples were loaded onto a 75 μm×50 cm PepMap RSLC EASY-Spray column filled with 2 μM C18 beads (ThermoFisher, San Jose, CA), operated at a pressure of 900 Bar and 60° C. Chromatography was performed using buffer A (0.1% formic acid v/v) and buffer B (80% acetonitrile, 0.1% formic acid v/v). Mass spectrometry data was processed using Proteome Discoverer version 2.5.0.400, with search parameters set to a parent mass error tolerance of 50 ppm, fragment mass error tolerance of 0.02 Da, max missed cleavages of 3. Fixed and variable modifications considered were carbamidomethylation: C (+57.02), oxidation: M (+15.99), deamidation: N, Q (+0.98), and acetylation: peptide N-term (+42.01). The data was searched against UniProt Bos taurus.

### Example 7: Results Related to Example 6

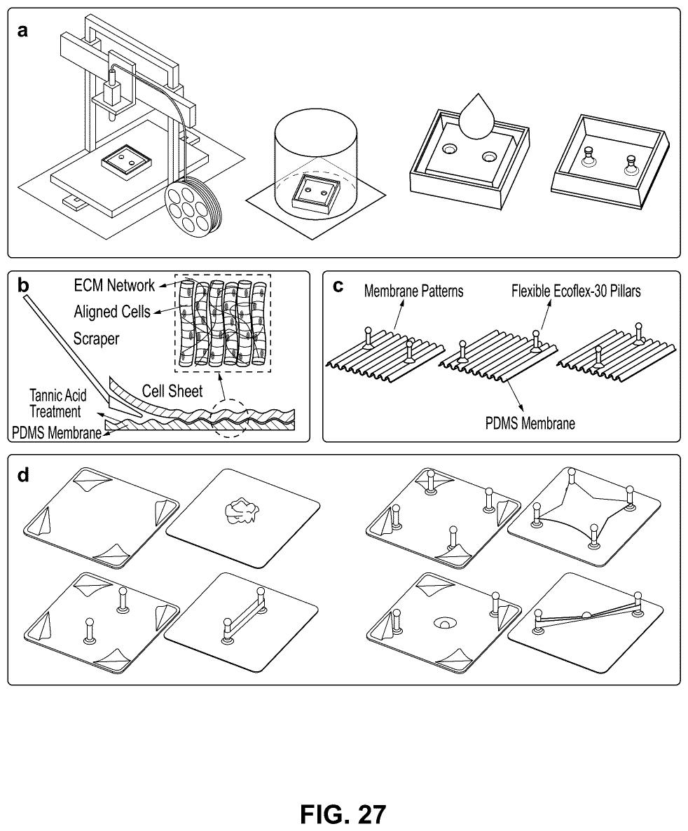

FIG. 27 shows an overview of aspects of a modeling platform based on cell sheet engineering, or a system for growing cell constructs. The method can use scaffold-free construction, in vivo-like ECM composition, and maintenance of cell-cell and cell-ECM junctions. In some embodiments, these features can be utilized for biofabricating 3D in vitro models, offering high physiological relevance for applications like drug discovery. The platform offers significant control over micro and macro structures, cell distribution and alignment, tissue construct form, and ECM production and arrangement. Moreover, it provides flexibility in model size and meets high-throughput requirements.

The culture devices for this novel cell sheet formation process employed PDMS as the base membrane for cell attachment, Ecoflex™ 00-30 pillars as anchors for 3D models, and a blend of PDMS and Ecoflex™ 00-30 for the culture device walls that contain the medium. First, 3D printed master molds with the inverse design of the culture device using ABS filament on a commercially available fused deposition modeling (FDM) printers were prepared. The 3D printed master molds had a surface texture composed of parallel lines with shallow grooves, which were replicated on the PDMS membrane of the culture device. These patterns served to induce desired features in the cell culture system, such as cell alignment and possible induced ECM production. Where these patterns were not needed, the surface finish of the master molds were treated with acetone vapor to remove undesired patterns. Devices with a full set of patterns were dubbed “Patterned” membranes. A 30-minute treatment with acetone vapor removed the parallel features, but maintained the grooves, resulting in “Minimally patterned” membranes that retained only 2.5D features created by the grooves.

Standalone cell sheets can be created using culture devices without pillars on the membrane. These are similar to cell sheets fabricated using other methods. However, unless stacked or integrated in vivo with target tissues, such cell sheets tend to collapse and form sphere-like aggregates due to uniform force distribution. The instability of these cell sheets, caused by loss of cell anchorage, was resolved by adding pillars protruding from the membrane. Cavities with the desired pillar shape were included in the master molds for culture devices. These cavities were filled with Ecoflex™ 00_30™ up to the pillar base, and the base was filled with a 1:1 volume mix of Ecoflex™ 00_30™ and PDMS. Then, the membrane region was cast with PDMS. These pillars gave new anchors to the cell sheets, allowing them to remodel into various 3D structures, dependent on the number and positioning of pillars. For instance, a 2-pillar system guided cells to remodel the sheets into hanging fiber-like constructs. In contrast, 4- or 6-pillar systems created stable hanging cell sheets. This system also allowed integration with other self-assembled, scaffold-free tissue constructs. such as multicellular spheroids, to form more complex structures. Moreover, by altering the positioning of pillars relative to the membrane patterns, cells could be guided to remodel the cell sheets and form anchored 3D constructs after detachment from the membrane.

The PDMS surface was rendered suitable for cell attachment through treatment with tannic acid. Tannic acid, a polyphenol, alters the hydrophilicity of PDMS permanently. The incorporation of shallow grooves on the membrane's surface, coupled with a 2.5D culture environment and chemical signaling through the addition of 2-Phospho-L-ascorbic acid to the medium, stimulated significant ECM production by cells. This environment facilitated the growth of multiple cell layers atop one another. As a result, these multi-layered cellular constructs established robust cell-cell and cell-ECM connections, which gradually weakened their attachment to the PDMS membrane surface. When they reached this stage, a simple scraping action could delaminate the cells and their ECM, yielding a self-assembled cell sheet.

### 7.1. Culture Device Fabrication and Cell Culture Treatment

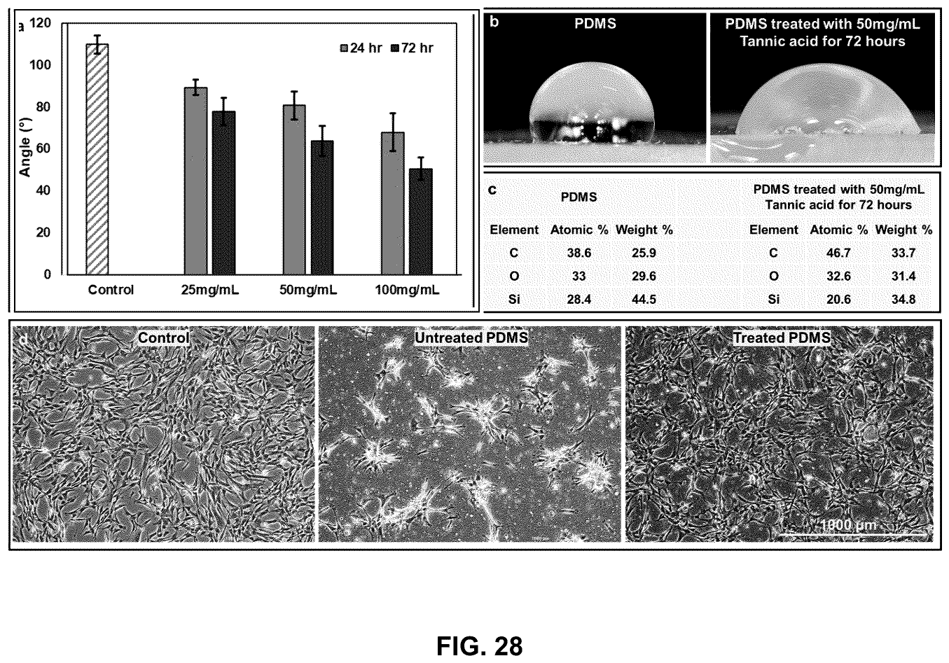

The culture devices' bottom membrane, used for cell attachment and growth, was fabricated using PDMS. Its wettability or hydrophilicity was adjusted with tannic acid treatment. The effects of both tannic acid solution concentration and treatment duration were investigated by treating PDMS with 25, 50, or 100 mg/mL tannic acid aqueous solution for either 24 or 72 hours. Contact angle measurements revealed the effects of different treatment modes on the PDMS surface's wettability. The treatment with a 50 mg/mL solution for 72 hours reduced the contact angle of untreated PDMS from 109.77±4.38 to 63.8±7.25° (FIGS. 28a and 28b). This condition was selected as the ideal treatment condition for subsequent experiment stages, and its effect remained stable even after autoclaving the culture devices (Data not shown).

Energy Dispersive Spectroscopy (EDS) was performed to investigate the treatment's effect on the surface's chemical composition, examining the elemental composition of both untreated PDMS and PDMS treated under ideal conditions. In both instances, the detector identified only oxygen, carbon, and silicone, but the percentage of detected silicone was notably lower in the treatment group, whereas the carbon percentage had increased (FIG. 28c).

The treatment's impact on cell attachment was evaluated by culturing SKMC on both untreated and tannic acid-treated PDMS, with tissue culture-treated 6-well plates serving as controls. While treated PDMS and the control group demonstrated appropriate cell attachment with no significant cell death, cells on untreated PDMS showed weak and insufficient attachment and spreading. They formed loosely attached clumps that detached from the membrane over time (FIG. 28d).

### 7.2. Cell Sheet Engineering

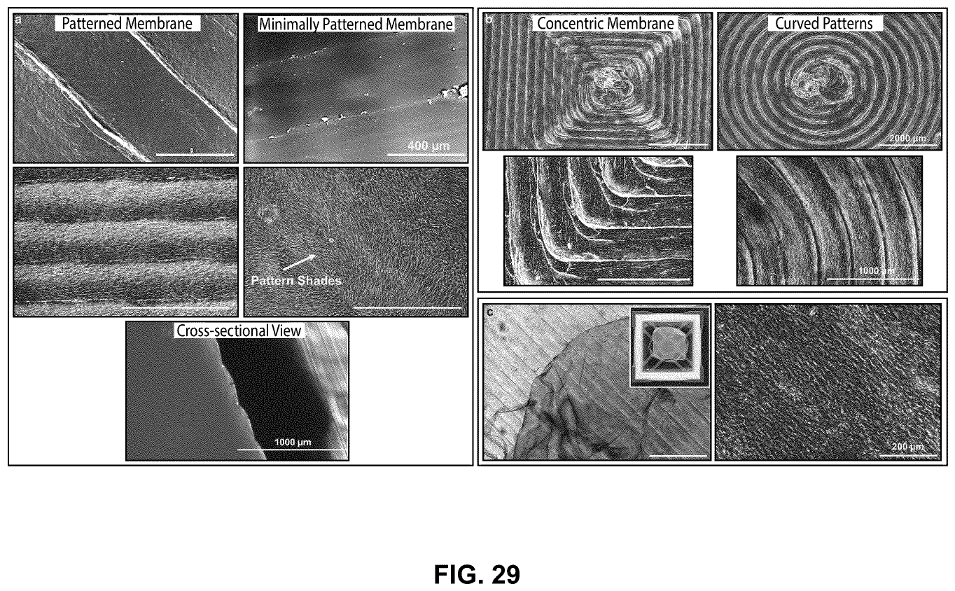

The culture devices were cast and fabricated using 3D printed master molds. A blend of PDMS and Ecoflex™ 00-30 in a 1:1 ratio was utilized for the device's walls, while pure PDMS was employed for the bottom membrane. FDM 3D printing created patterns on the master molds' surface, which were replicated on the PDMS surface and aided in cell alignment. Surface finishing was carried out by treating the ABS master molds with acetone vapor to fade or completely erase the patterns, resulting in minimally patterned membranes. SEM images (FIG. 29a) show patterned and minimally patterned PDMS membranes, along with their effects on cell alignment. The 3D printing process typically uses a monotonic fill pattern for the surface, but other patterns, such as concentric or curved ones, can also be utilized (FIG. 29b). These patterns, often deemed artifacts of the 3D printing process, can effectively guide cell alignment in cells sensitive to environmental signals, like skeletal muscle cells.

In order to create self-assembled cell sheets without stimuli-responsive culture surfaces or triggers for cell layer detachment, multiple cell layers were grown on top of each other. Cells were induced to produce more ECM via the inclusion of 2-Phospho-L-ascorbic acid. SKMCs were cultured to reach >90% confluency by day 2. The medium was changed to a differentiation medium on day 3 and refreshed every other day. Additional cells were added on days 4, 7, and 10, with culture continuing until day 18. As cells established stronger cell-cell and cell-ECM junctions, their membrane attachment weakened. By day 18, scraping and detaching the ECM-rich layer from the culture device edges was sufficient to delaminate the entire layer as a self-assembled cell sheet (FIG. 29c), which was durable enough for manual handling. Following detachment, cells could sustain their alignment. It is noteworthy that cell sheets could be formed any time after day 12, but they were the strongest on day 18, beyond which spontaneous detachment might occur.

7.3. 3D Construct Formation with Cell Sheets as Building Blocks

Cell sheets, while offering preserved cell-cell and cell-ECM connections, often tend to form sphere-like constructs due to uniform force distribution after losing their anchors (FIG. 30a). FIG. 30, panel a, shows a cell sheet that was detached from a membrane without protuberances. Cell incubation continued for 48 hours. Over this time, the cell sheet folded and formed an agglomerate.

To guide cellular behavior, including remodeling, a range of Ecoflex™ 00-30 pillars were designed to protrude the PDMS membrane in various patterns. Pillars were fused at the base using a 1:1 mix of PDMS and Ecoflex™ 00-30. Once cell layers were ready to detach as a sheet, a scraper was used to scrape the layer to the shaft of the pillars. Subsequently, a 1000 μL pipette tip gently detached the area between the pillars. Providing only two pillars guided the 2D sheets to transition into 3D anchored fibers (FIG. 30b). Depending on the positioning of these pillars (diagonally or at the center of the culture device edges), fibers of varying thickness were formed. The creation of 4- and 6-pillar systems resulted in anchored, hanging sheets. Unlike standalone sheets, the anchored sheets showed only minor consolidation after delamination, maintaining their size even over long-term cultures (FIGS. 30c and 30d). More anchors in different directions further reduced sheet shrinkage in certain directions, such as the reduced y-axis shrinkage in the 6-pillar system compared to the 4-pillar system.

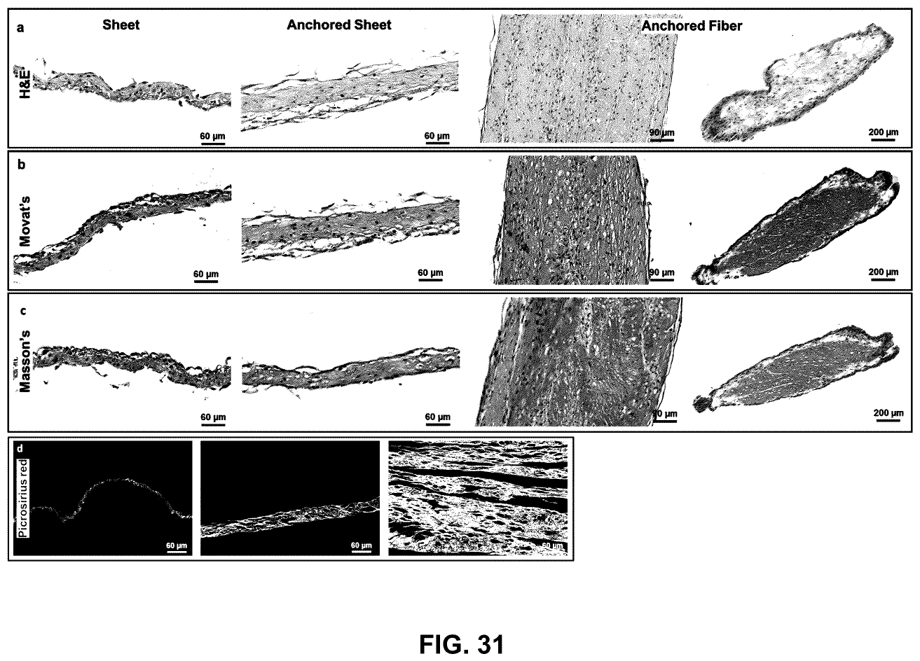

To better understand the composition and cell distribution of these constructs, histology staining was conducted using Hematoxylin and Eosin (H&E), Movat's Pentachrome, Masson's Trichrome, and Picrosirius Red. Staining was performed for standalone sheets immediately post-delamination, anchored sheets after 2 days, and anchored fibers after 4 days (FIG. 31). Despite only 2 additional days in culture, anchored sheets showed significantly increased ECM content. Similarly, anchored fibers also showed a significant presence of ECM components, such as collagen and elastin.

7.4. Integration of 3D Spheroids with Sheets

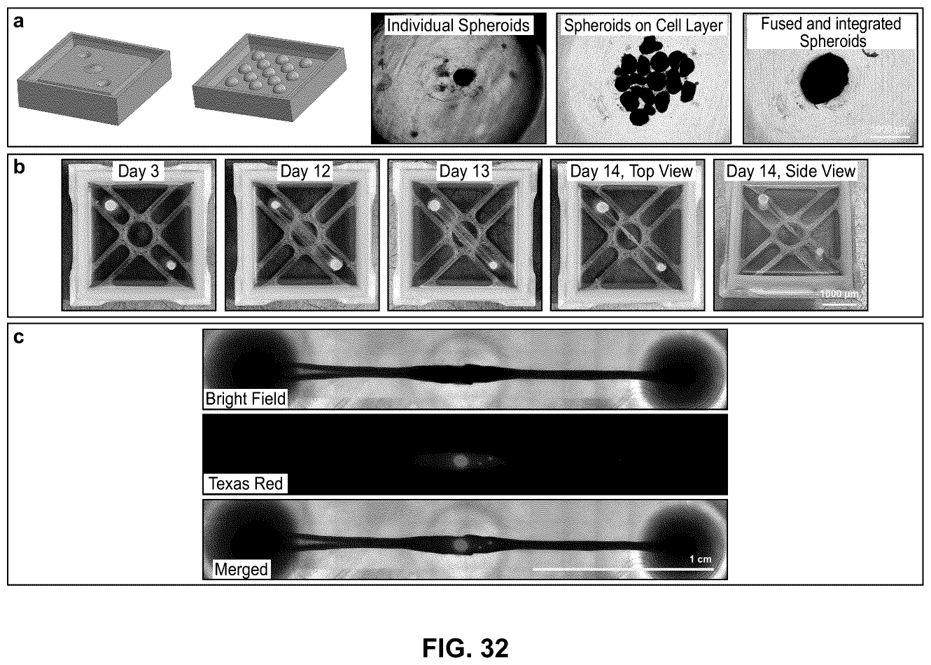

To recreate more complex in vivo structures, integration of tissues with varying form factors is essential. This versatility was shown by enabling the integration of scaffold-free spheroids with 2D cell cultures, which eventually transition to a 3D anchored fiber incorporating the spheroids. Spheroids were generated using a PDMS culture device with semi-sphere indentations acting as wells (FIG. 32a). To increase cell repellency, the device was treated with Pluronic F127. Post 24 hours of adding 3×10⁶ SKMCs stained with Nile red, spheroids formed and were transferred to a tannic acid-treated culture device with a confluent monolayer of cells on day 2 (FIG. 32b). A small semi-sphere well was included in this design to hold the spheroids while they integrated with the cell monolayer. Over 2 days, smaller spheroids consolidated into a larger one and fused with the 2D cell layer. The culture process mimicked previous steps, including the addition of more cells and treatment with differentiation medium containing 2-Phospho-L-ascorbic acid for enhanced ECM production. By day 12, cell sheet formation began, and by day 14, the muscle fiber formed with the spheroid engulfed in it. Fluorescent microscopy was used to visualize the position of the Nile red-stained spheroid within the new fiber (FIG. 32c).

### 7.5. Air Liquid Interface Culture System

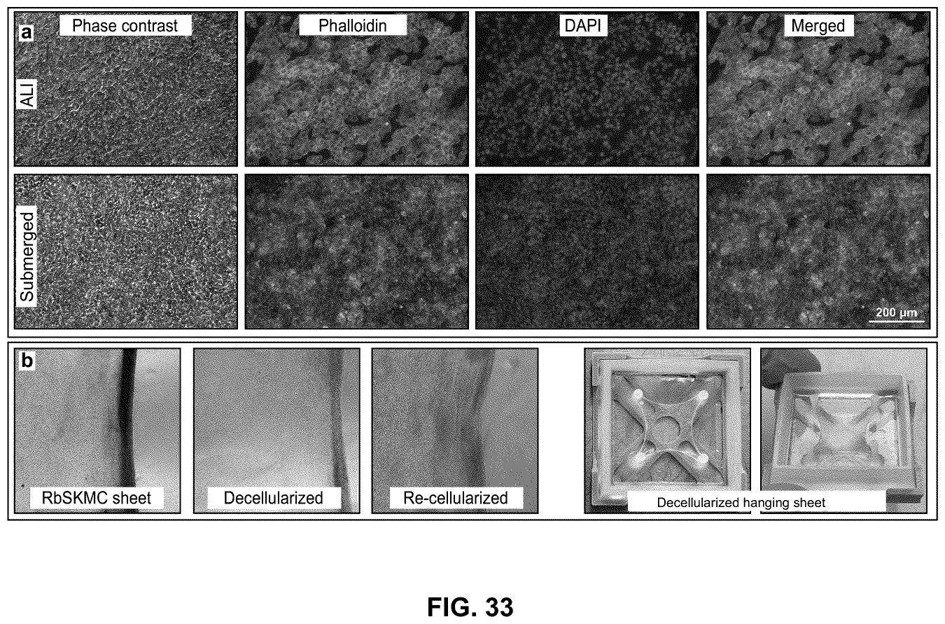

in vitro culture conditions often immerse cells in media, while some in vivo situations expose cells to gaseous environments, establishing baso-apical morphologies. To mimic this, hanging sheets were formed in a 4-pillar system using SKMC cells. These were then decellularized for 48 hours at 4° C. with a detergent solution, followed by sterilization and extensive washing to remove any detergent or ethanol residues. Next, the sheets were recellularized with a large number of A549 cells. After 30 minutes of incubation, the cells demonstrated proper attachment to the ECM sheet. Within another 24 hours in the incubator, they formed a uniform, confluent layer. At this stage, we split the cultures into two groups. One maintained the submerged condition, while the other was transitioned to an air-liquid interface (ALI) state by reducing the medium volume, enabling the ECM sheet and cells to float. After ten days, Phalloidin and DAPI staining revealed distinct morphologies in the two conditions. ALI-conditioned cells exhibited larger morphologies and better mimicked in vivo-like epithelial layers, whereas the submerged cells were smaller and flatter (FIG. 33).

### 7.6. Decellularized Cell-Sheet Constructs as Scaffolding

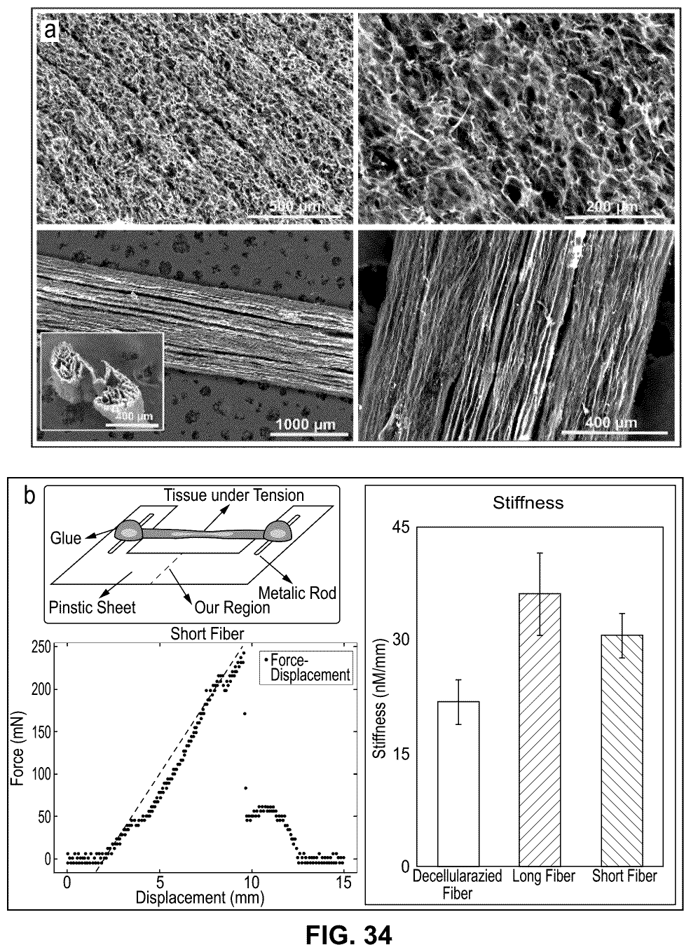

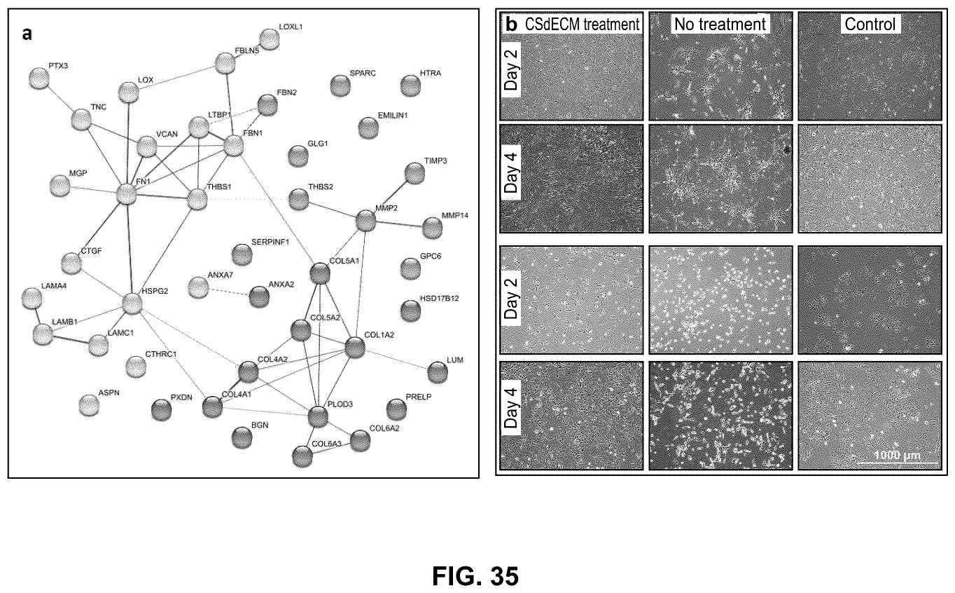

The potential of decellularized cell sheets and anchored fibers as sources of rich extracellular matrix (ECM) scaffolds was assessed using SEM, revealing abundant ECM content (FIG. 34a). Decellularized sheets displayed some ECM alignment, while anchored fibers demonstrated significantly higher alignment, indicative of cell-mediated remodeling guided by pillar directionality. Decellularized anchored fibers revealed dense, ECM-rich constructs in cross-section.

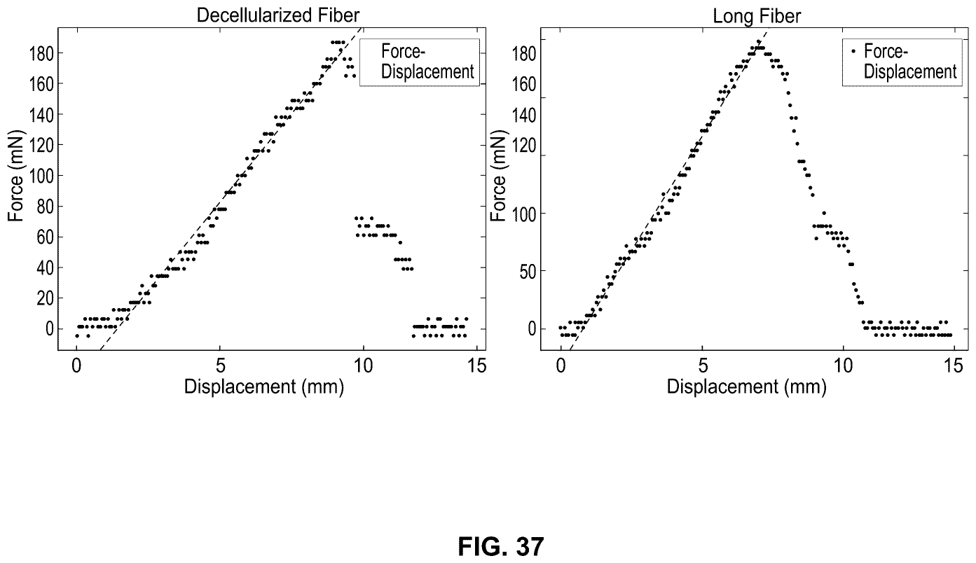

Mechanical testing was performed on uniform 5 cm long anchored fibers and their decellularized versions as well as nonuniform 2 cm long fiber formed using diagonally placed pillars. It demonstrated that ECM contributes significantly to the mechanical strength of fibers, with stiffness decreasing post-decellularization. Interestingly, non-uniform fibers displayed reduced stiffness compared to their uniform counterparts (FIG. 34b). This suggests that fiber form factors may influence their mechanical properties. Behavior of short non-uniform fibers under tensions is shown as an example here and FIG. 37 shows behavior of uniform long fiber and its decellularized version.

### 7.7. Cell Sheet-Derived ECM (CSdECM) as Coating

The proteomic analysis of the cell sheets produced a comprehensive profile of the ECM proteins, with 1382 proteins identified in total. Of these, ECM components were analyzed more deeply, as their abundance signifies the successful enrichment of ECM in the cell sheets and potential utility in other applications. Using the STRING-db for network analysis, a subset of 44 ECM-related proteins out of the identified 344 cellular components was selected. This subset was evaluated for physical interactions and clustered into three distinct groups, demonstrating the richness and diversity of the ECM composition in our cell sheets (FIG. 35a). The physical interactions between these ECM components were further substantiated by varying confidence levels, indicated by the thickness of the edges between proteins. The resulting network provides strong evidence for the existence of protein complexes within the ECM composition. Detailed description of detected proteins is included in Table 1.

Cell sheets, with their abundant extracellular matrix (ECM), were utilized to improve in vitro cultures by converting them into a CSdECM solution. Firstly, cell sheets from SKMCs were produced and decellularized. These decellularized sheets were solubilized using a high ionic strength solution containing 3M Urea and 0.1M acetic acid in PBS. This solution was further dialyzed and treated with DNase to remove any DNA remnants. The CSdECM solution was then applied to non-treated 6-well plates, which were subsequently used for the culture of SKMCs and A549 lung cells. Control groups included wells without CSdECM treatment and conventional tissue-culture treated wells. Phase contrast microscopy revealed that CSdECM enhanced both cell attachment and growth. On CSdECM treated surfaces, both SKMCs and A549 cells exhibited faster proliferation and more pronounced cell morphologies when compared to the control groups. SKMCs also showed differentiated morphologies faster.

### 7.8 Tensile Strength

FIG. 37 shows the results of tensile strength testing of uniform 5 cm long fiber and its corresponding decellularized ECM fiber.

7.9 Effect of Treatment with Lignin

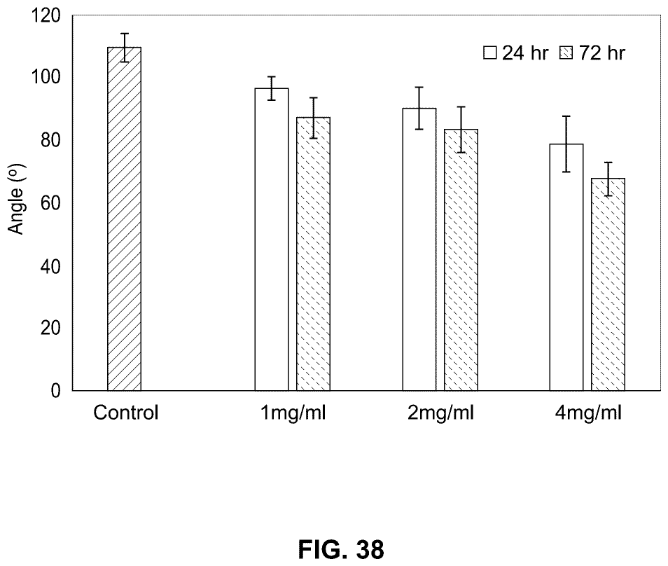

FIG. 38 shows the effect of treatment with lignin, a natural polyphenol, on PDMS wettability. PDMS membranes were exposed to aqueous solutions of lignin (obtained from Sigma-Aldrich, Catalog #471003) for durations of either 24 or 48 hours, after which contact angle measurements were performed.

### 7.10 Impact of Membrane Patterns

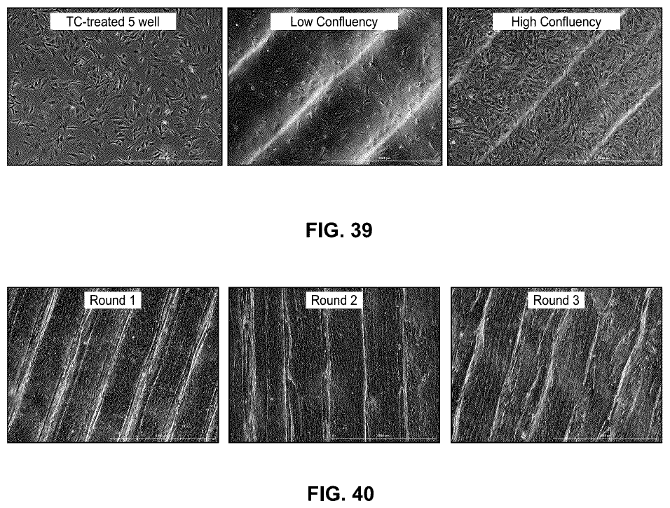

FIG. 39 show the impact of membrane patterns on the attachment and alignment of primary bovine myoblasts. Interestingly, the morphology and alignment of bovine myoblasts were not influenced by the membrane patterns, in contrast to the SKMCs. However, these cells still exhibited appropriate attachment to the membrane surface. Bovine myoblasts were grown in DMEM (Sigma Aldrich, Catalog #D5796) supplemented with 10% FBS (Sigma Aldrich, Catalog #F4135). The cells were grown to confluence and subsequently dissociated using trypsin (Gibco, Catalog #R001100).

### 7.11 Reuse of Culture Devices

FIG. 40 shows the reuse of culture devices following wash with 2-Propanol and autoclaving. Despite the rigorous cleaning process involving washes with 2-Propanol and subsequent autoclaving, no additional tannic acid treatments are necessary beyond the initial application. This durability, coupled with straightforward maintenance, makes these culture devices not only cost-effective, but also user-friendly for scientific applications.

### 7.12 Use of Commercial Cell Sheet Engineering Substrate

FIG. 43 shows the formation of anchored cell sheet engineering constructs using other cell sheet engineering platforms facilitated by attaching Ecoflex™ 00-30 pillars to the lid of temperature-sensitive Nunc® UpCell™ Surface cell culture 12 well plates (Sigma-Aldrich, product number Z688797). These pillars are held in place using magnets. Shown are the bottom views of SKMC sheets released at room temperature both without any pillars and with varying numbers and configurations of pillars. In the absence of pillars, the sheets tend to collapse on themselves, while the presence of pillars helps in maintaining stable, defined shapes for the sheets. A single layer of SKMCs was cultured in each well. Following 4 days of differentiation, the pillars were installed and the sheets were detached. The pillars were designed to maintain a distance of 0.1 mm from the well surface, where the cells were cultivated.

### 7.13 Network Visualization

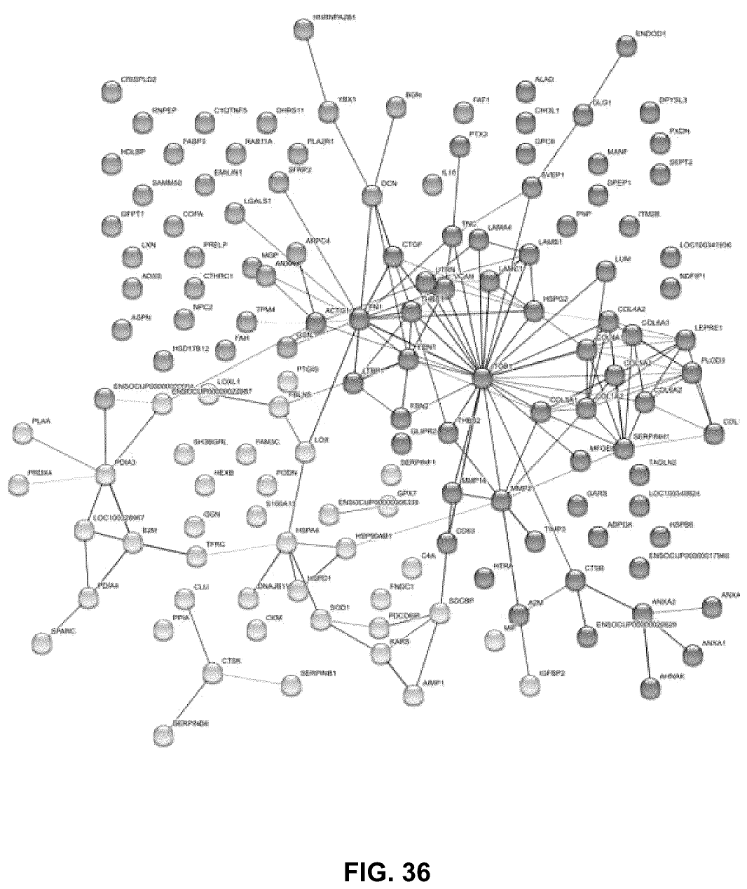

FIG. 36 shows a network visualization of broader extracellular space components identified from the SKM sheets. The network represents proteins identified in the ECM space (n=139 of 2270 proteins from cellular component gene ontology analysis). Node colors represent different protein clusters, and line thickness indicates the strength of data support for each interaction.

TABLE 1
Full names and brief descriptions of
the proteins represented in FIG. 36.

| | |
|---|---|
| SERPINF1 | Pigment epithelium-derived factor; Serpin family F |
| | member 1; Serpin peptidase inhibitor, clade F |
| | (alpha-2 antiplasmin, pigment epithelium derived |
| | factor), member 1 (418 aa) |
| COL6A2 | Collagen, type VI, alpha 2 (981 aa) |
| LOXL1 | Lysyl oxidase-like 1 (542 aa) |
| VCAN | Versican core protein; Versican (3361 aa) |
| CTGF | Connective tissue growth factor (349 aa) |
| LOX | Protein-lysine 6-oxidase; Lysyl oxidase (405 aa) |
| GLG1 | Golgi apparatus protein 1; Golgi glycoprotein 1 |
| | (1225 aa) |
| THBS1 | Thrombospondin 1 (1171 aa) |
| FBN1 | Fibrillin 1 (2821 aa) |
| ANXA2 | Annexin A2 (346 aa) |
| LUM | Lumican (338 aa) |
| MMP2 | Matrix metalloproteinase-2 (gelatinase a) (662 aa) |
| EMILIN1 | Elastin microfibril interfacer 1 (1012 aa) |
| COL6A3 | Uncharacterized protein; Collagen, type VI, alpha 3 |
| | (1684 aa) |
| FBN2 | Fibrillin 2/3; Fibrillin 2 (2912 aa) |
| TIMP3 | Timp metallopeptidase inhibitor 3 (211 aa) |
| CTHRC1 | Collagen triple helix repeat containing 1 (239 aa) |
| LTBP1 | Latent transforming growth factor beta binding |
| | protein 1 (1520 aa) |
| LAMA4 | Laminin subunit alpha 4; Laminin, alpha 4 (1817 aa) |
| PRELP | Proline/arginine-rich end leucine-rich repeat protein |
| | (380 aa) |
| FN1 | Fibronectin 1 (2427 aa) |
| PTX3 | Pentraxin 3, long (381 aa) |
| MMP14 | Oryctolagus cuniculus matrix metallopeptidase 14 |
| | (membrane-inserted) (mmp14), mrna. (582 aa) |
| SPARC | SPARC (305 aa) |
| COL1A2 | Collagen alpha-2(I) chain; Type I collagen is a |
| | member of group I collagen (fibrillar forming |
| | collagen) (1364 aa) |
| ANXA7 | Annexin a7/11; Annexin A7 (487 aa) |
| LAMC1 | Laminin subunit gamma-1; Uncharacterized protein |
| | (983 aa) |
| FBLN5 | Fibulin 5 (448 aa) |
| COL4A1 | Collagen, type IV, alpha 1; Belongs to the type IV |
| | collagen family (1667 aa) |
| COL4A2 | Collagen, type IV, alpha 2; Belongs to the type IV |
| | collagen family (1700 aa) |
| HTRA | HtrA serine peptidase 1 (357 aa) |
| GPC6 | Uncharacterized protein; Cell surface proteoglycan |
| | that bears heparan sulfate (182 aa) |
| LAMB1 | Laminin subunit beta 1; Laminin, beta 1 (1782 aa) |
| COL5A2 | Collagen type v/xi/xxiv/xxvii, alpha; Collagen, |
| | type V, alpha 2 (1502 aa) |
| MGP | Matrix Gla protein; Associates with the organic |
| | matrix of bone and cartilage. Thought to act as |
| | an inhibitor of bone formation (103 aa) |
| HSD17B12 | 17beta-estradiol 17-dehydrogenase/very-long-chain |
| | 3-oxoacyl-CoA reductase; Hydroxysteroid (17-beta) |
| | dehydrogenase 12 (283 aa) |
| HSPG2 | Basement membrane-specific heparan sulfate |
| | proteoglycan core protein; Heparan sulfate |
| | proteoglycan 2 (4392 aa) |
| COL5A1 | Uncharacterized protein; Collagen, type V, alpha 1 |
| | (590 aa) |
| PLOD3 | Procollagen-lysine, 2-oxoglutarate 5-dioxygenase 3; |
| | Procollagen-lysine, 2-oxoglutarate 5-dioxygenase 3 |
| | (581 aa) |
| TNC | Tenascin C (2384 aa) |
| PXDN | Peroxidase; Peroxidasin homolog (Drosophila) |
| | (1411 aa) |
| THBS2 | Thrombospondin 2 (1125 aa) |
| ASPN | Asporin (373 aa) |
| BGN | Biglycan (394 aa) |

### 7.14 Discussion

The devices described herein use a trigger-free cell sheet engineering technique, relying on cells' capacity to form robust cell-cell junctions and interactions with their ECM network, weakening their adhesion forces to the culture surface. A straightforward scraping action can then detach the cells and their ECM as a cohesive, robust sheet (FIG. 29). Typically, cell adhesion to any surface is facilitated through integrin adhesion mechanisms, capable of regulating cytoskeletal structure via transmembrane components. Both passive and cell-mediated regulation of cell-substrate interaction can lead to the detachment of the cell layer as a sheet, without the necessity for external stimuli. In order to influence and weaken the cells' attachment to the culture substrate as well as improving the integrity of the cell sheet layer, in this work, multiple cell layers were sequentially grown on top of each other over a predetermined timeframe.

PDMS, chosen as the resin for fabricating the culture devices in some embodiments, has properties including biocompatibility, optical transparency, flexibility, durability, and ease of fabrication. However, its inherent hydrophobic nature and lack of recognizable functional groups render it unsuitable for cell attachment. As described herein, polyphenols, such as tannic acid (FIG. 28) and lignin (FIG. 38), can directly influence the PDMS surface contact angle, thereby facilitating cell adhesion. Tannic acid proved more effective in reducing the PDMS's contact angle. Among the various tested conditions, treatment with a 50 mg/mL aqueous solution of tannic acid for 72 hours achieved a contact angle of 63.8±7.25°, chosen as an exemplary condition (FIGS. 28a and 28b) which is within optimal range of 50-80° for cell attachment. Adsorption of tannic acid on the PDMS membrane surface was evident by the decrease in the total amount of silicon detected and the increase in carbon, a key component of tannic acid, as determined by EDS (FIG. 28c). The tannic acid treatment alone proved sufficient for proper cell attachment and spreading on the PDMS membrane compared to the tissue culture polystyrene controls (FIG. 28d).

The fabrication of culture devices used for casting PDMS involved preparing master molds using 3D printing. Surface patterns, often perceived as artifacts of the 3D printing process, were shown to have the capacity to align cells (FIG. 29). Open-source 3D printing was used for efficient cell alignment (FIG. 29a). The surface roughness intrinsic to 3D printing processes, mirrored on the PDMS surface, might have also facilitated cell attachment. While cell alignment is beneficial for several applications, such as skeletal muscle, heart, vascular smooth muscle cells, and neuronal tissues, it may not be necessary for others. To eliminate patterns on 3D printed master molds, printed parts (using ABS filaments) underwent treatment with gaseous acetone under controlled conditions, largely eliminating patterns and resulting in minimally-patterned finishes (FIG. 29a). Regardless of 3D printing patterns or acetone vapor finishing, the cross-section of all PDMS membranes incorporated shallow grooves, enabling growth of multiple cell layers on top of each other, and creating a pseudo-3D (2.5D) culture environment. When cells not sensitive to external alignment signals, such as fibroblasts, were used on patterned membranes, their morphology was not affected (FIG. 39). The directionality of the patterns could be manipulated by altering the 3D printing direction, which was interpreted by the cells (FIG. 29b). This cell directionality was maintained post-delamination of the sheets from the membranes (FIG. 29c). It was demonstrated that culture devices could be reused post-sheet detachment following washing and autoclaving steps (FIG. 30) without negatively impacting cell behavior or causing membrane turbidity that could interfere with microscopy. This reusability potentially reduces the overall cost of modeling by requiring fewer devices.

Without adequate anchors, constructs can't resist active cellular forces, leading to agglomeration of 2D sheets, which limits their use for in vitro modeling (FIG. 30a). This limitation can be mitigated by providing cell sheets with anchors to resist cellular forces and facilitate remodeling into desired 3D constructs. This was achieved in some embodiments by adding biocompatible pillars made from the flexible Ecoflex™ 00-30, a poly(1,4-butylene terephthalate) (PBAT)-based elastic biocompatible resin, to the transparent PDMS culture surface (FIG. 30b-d). The pillars' presence influenced how cell sheets remodeled to form hanging fibers or sheets (FIG. 30b-d). The pillar count and placement, controlled cell resistance thereby directing remodeling. Tissue constructs in FIG. 30 were formed using SKMCs, demonstrating their capability to exert contractile forces and contract sheets. Other cell types showed similar behavior, such as human-induced pluripotent cells (iPSCs) differentiated into hepatocytes (FIG. 41). Depending on the initial surface area, tissue constructs of various sizes can be biofabricated (FIG. 42). This pillar anchoring strategy can be adapted for any cell sheet engineering technique, as illustrated with temperature-sensitive poly(N-isopropylacrylamide) coated well plates (FIG. 43). This modification could enhance scaffold-free in vitro model biofabrication using any cell sheet engineering technique.

Histological analysis revealed significantly higher cell densities and ECM components in hanging sheets and fibers compared to standalone sheets immediately post-delamination (FIG. 31). The abundance of ECM and its remodeling within these constructs were further corroborated by SEM images of decellularized sheets and anchored fibers (FIG. 34a). Cell and ECM alignment was not only observed on patterned membranes (FIG. 29) but also was significantly enhanced in anchored fibers after 2D sheets transitioned into 3D constructs. The fibers exhibited robust integrity. A comparison of the mechanical properties of these fibers and their decellularized ECM revealed that the decellularized fibers, comprised solely of ECM, still maintained substantial mechanical strength (FIG. 34b and FIG. 37.

In some embodiments, constructs can be integrated into more representative models, referred to as assembloids. As shown in FIG. 32, the integration of 3D spheroids, formed by the self-assembly of fluorescently stained SKMCs, was achieved. These 3D constructs could successfully integrate with the 2D culture of cells on the PDMS surface, and eventually become part of the hanging sheet formed. Potential applications of such constructs include the integration of central nervous system models with peripheral nervous system models.

Decellularized cell sheet-derived components offer versatile applications, including serving as substrates for other cell types. For instance, decellularized anchored sheets were used to establish an air-liquid interface (ALI) state for lung epithelial cells (FIG. 33). After forming anchored sheets with SKMCs and decellularizing them, lung A549 cells were cultivated on these ECM membranes. After just 10 days in culture, these cells displayed different morphologies in submerged and ALI conditions, indicating the potential for recreating complex tissue structures.

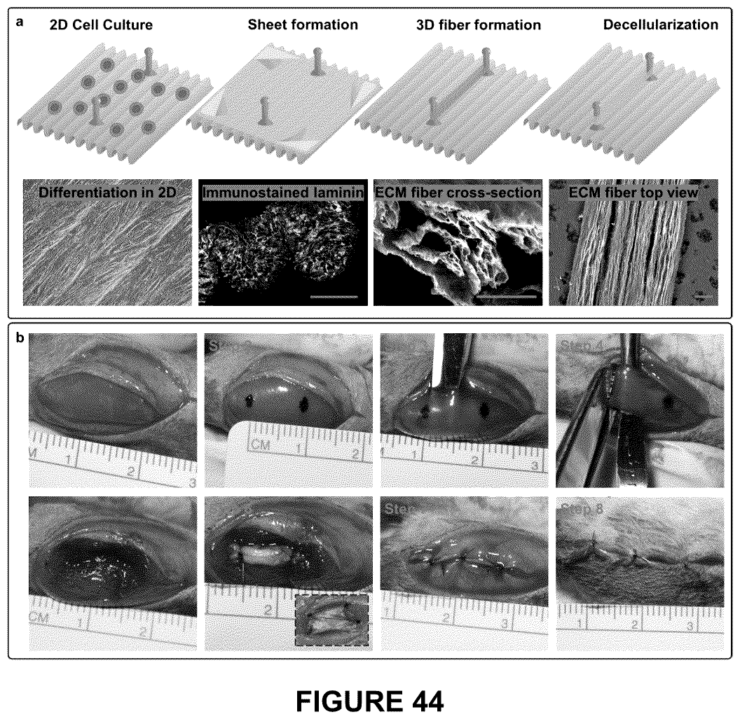

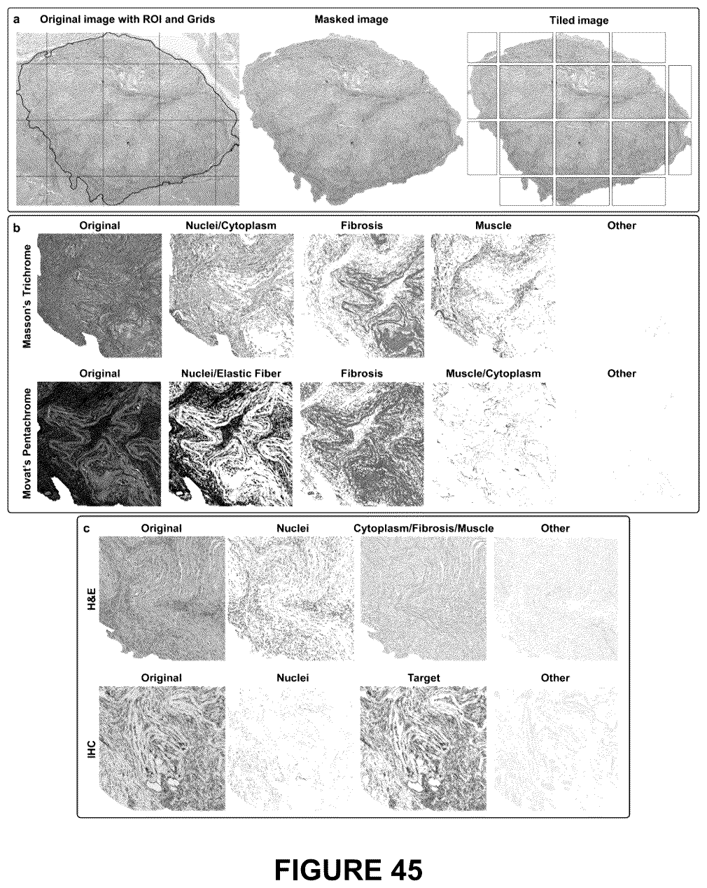

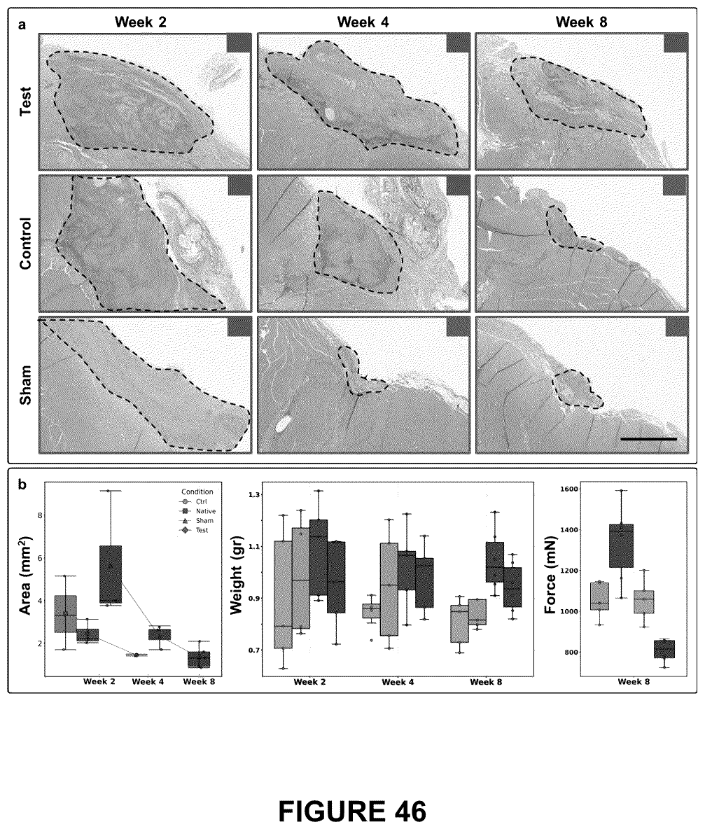

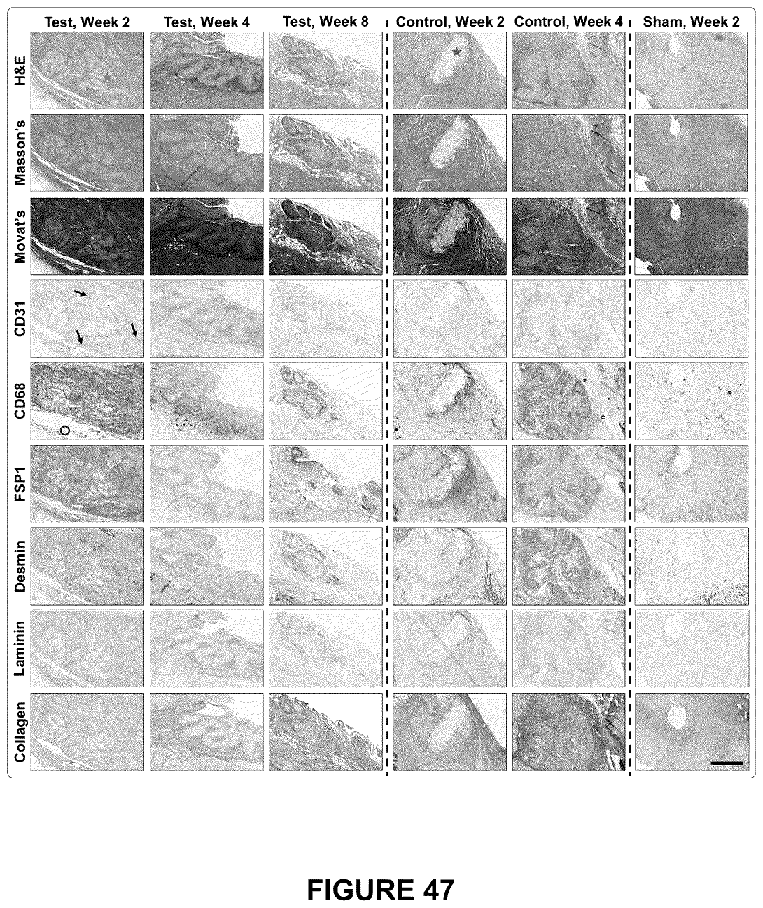

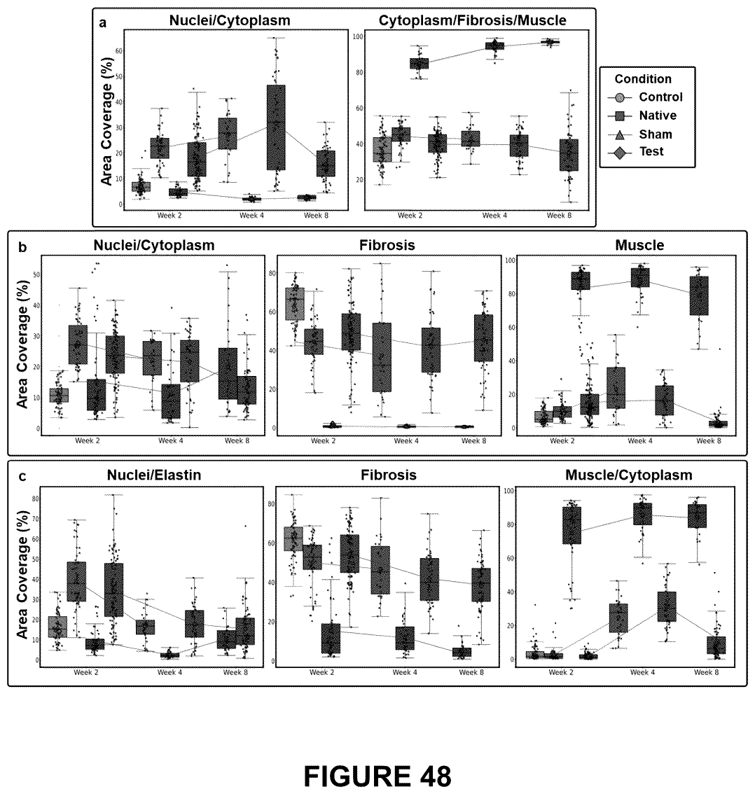

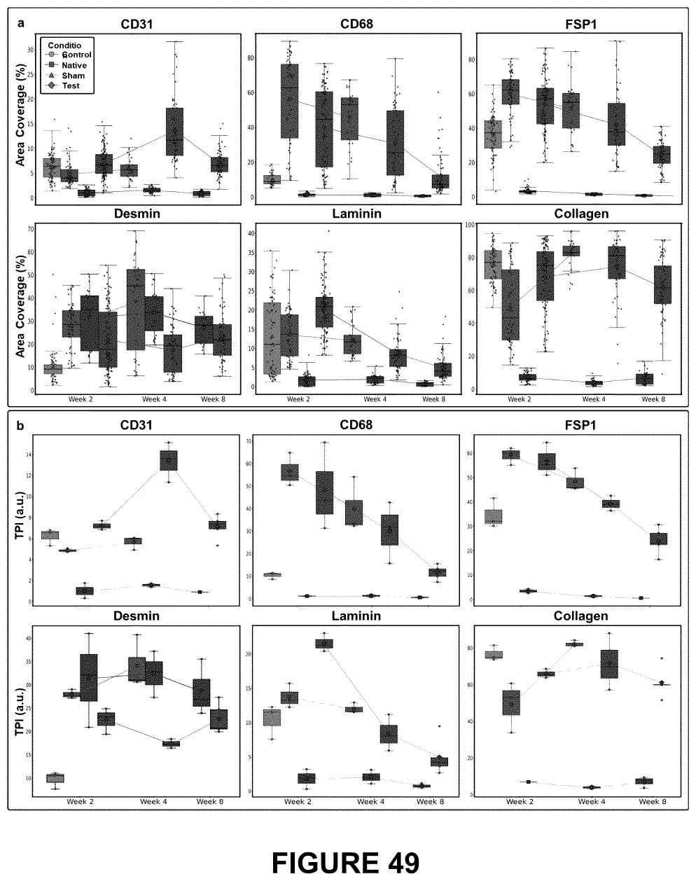

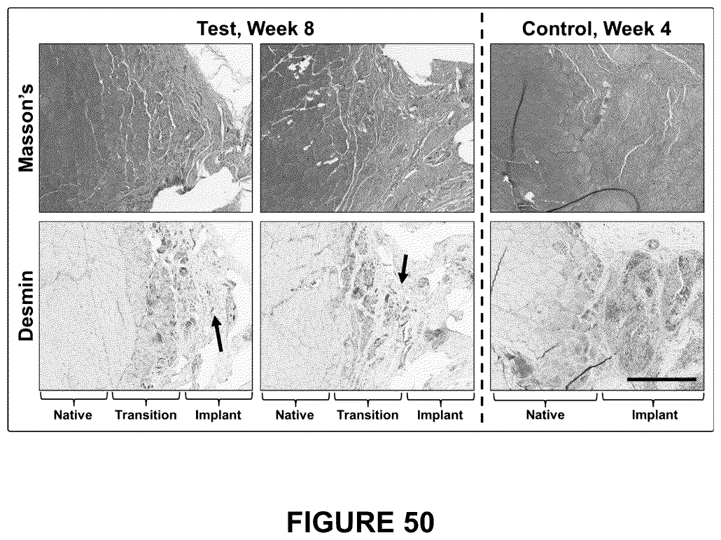

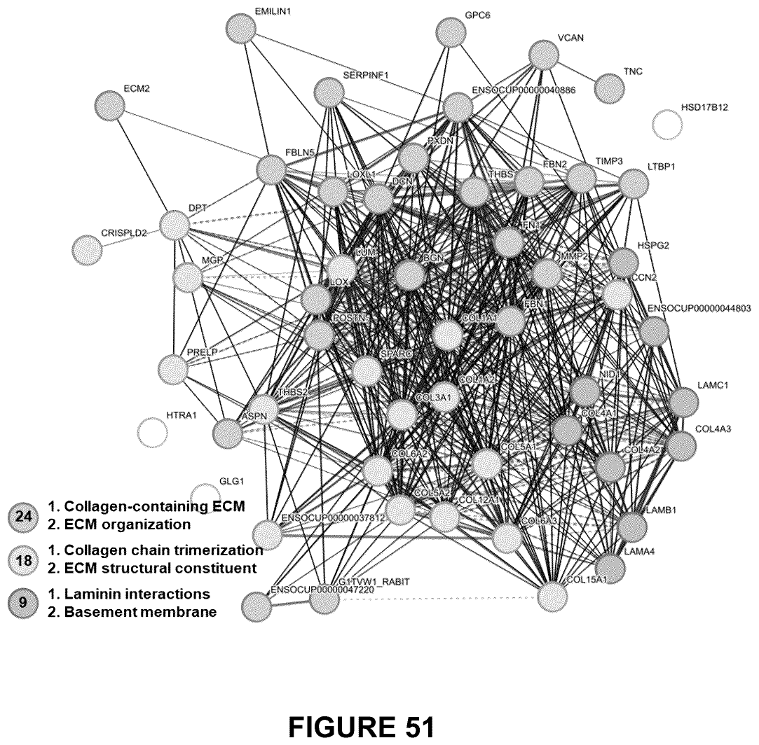

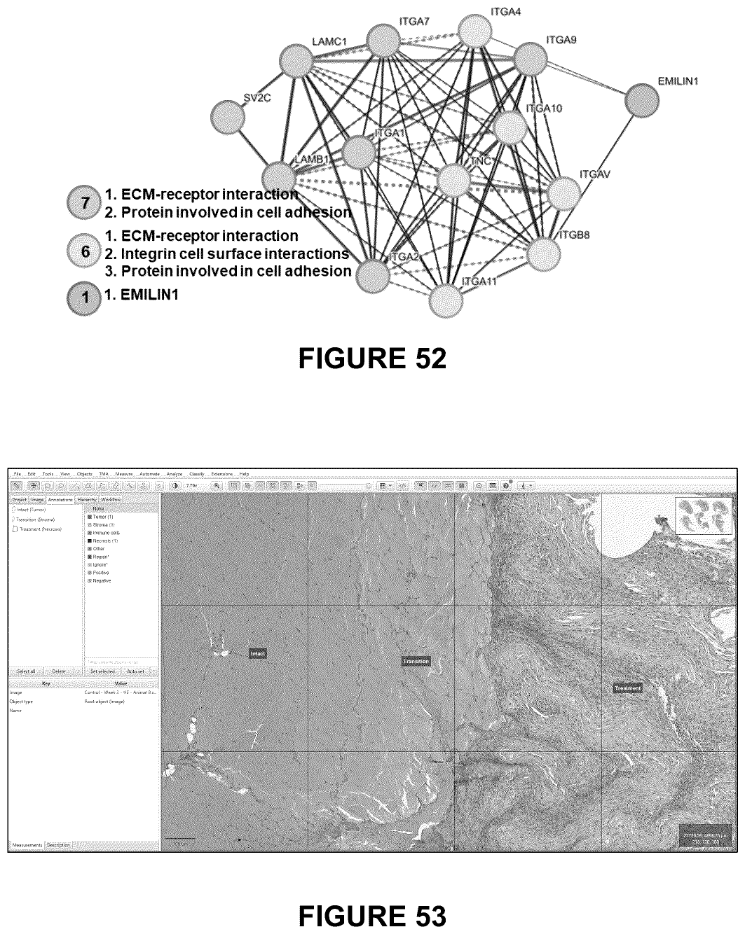

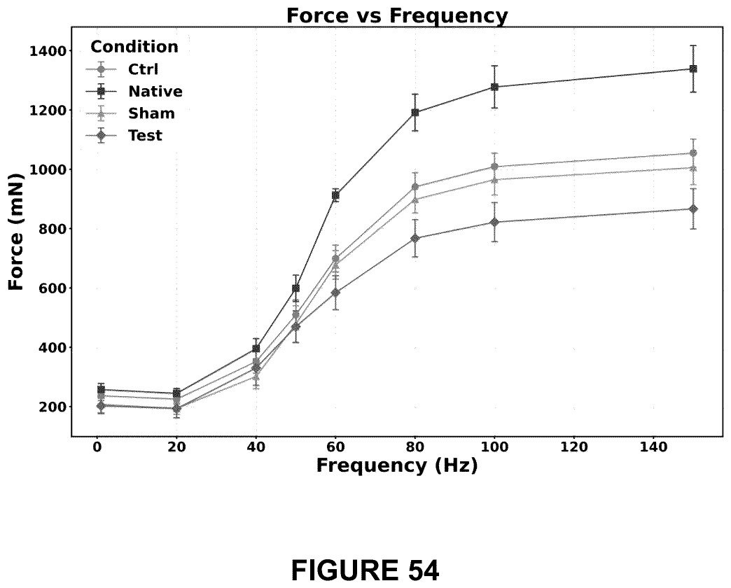

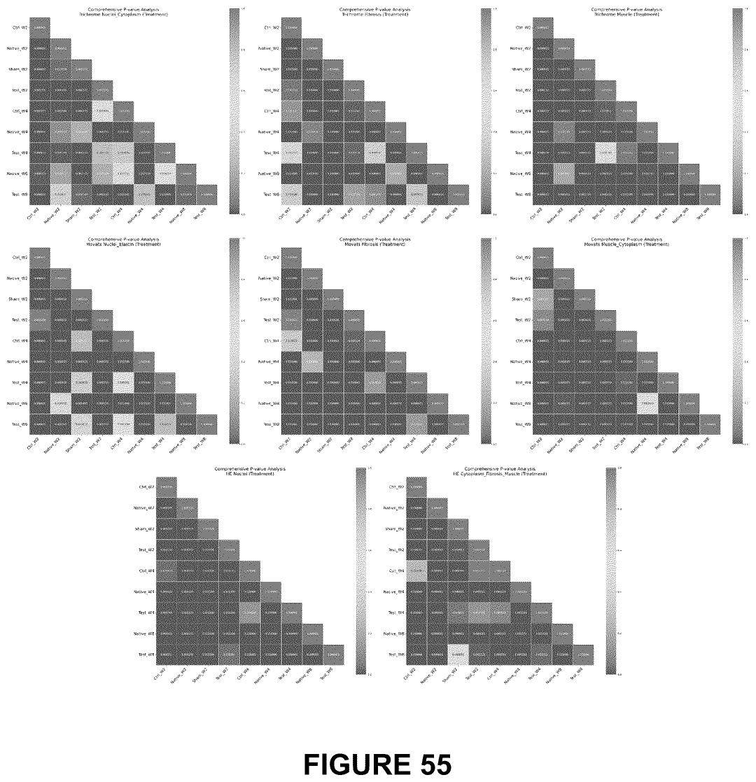

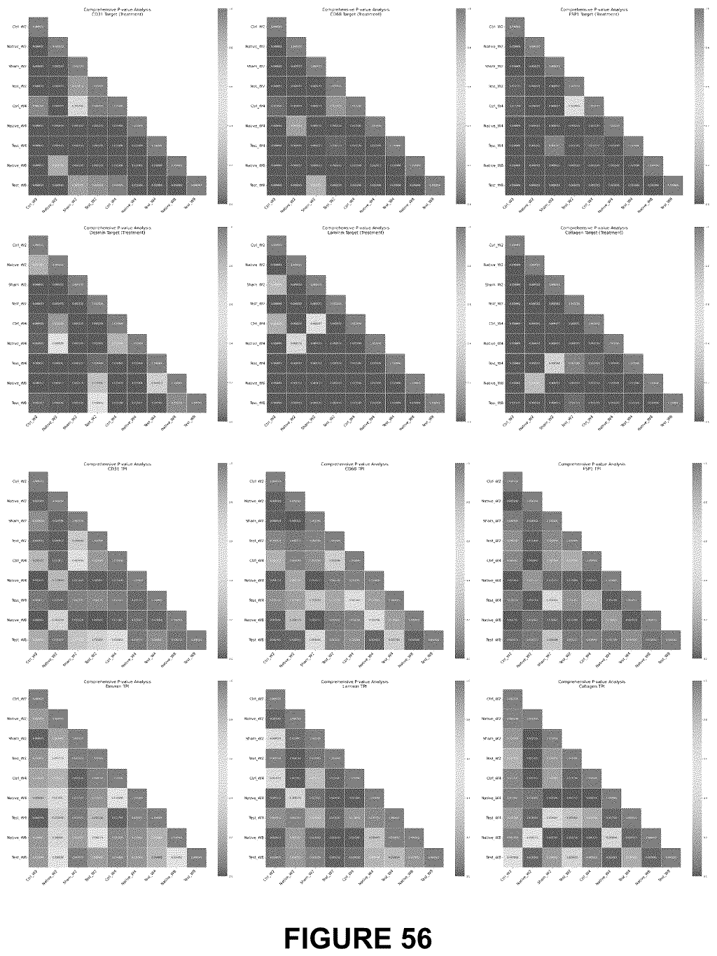

The rich and varied composition of ECM proteins within the cell sheets was revealed through proteomics analysis (FIG. 35a). The broad range of ECM-related proteins were identified via gene ontology and network analysis showed the extensive interactions among the proteins within the ECM. The CSdECM could create a biomimetic platform to support more physiological cell behavior. In addition to the core ECM components identified, further exploration of the proteomic data highlighted an array of additional extracellular space components (FIG. 44). These elements potentially contribute to the extensive interplay between cells and their matrix, thereby facilitating a more conducive environment for cell adhesion and function. Some examples of these components include key regulatory molecules like galectin, various types of collagens (types V, XI, XXIV, XXVII among others), and annexins (A2, A5, A7).

The potential of using CSdECM to establish a rich and in vivo-like surface treatment for 2D culture systems was demonstrated (FIG. 35b). CSdECM derived from SKMCs was used as a coating for both fresh SKMCs and lung A549 cells. In both instances, cells exhibited faster proliferation on non-tissue culture wells treated with CSdECM compared to tissue culture-treated wells without any CSdECM treatment. This was contrasted by both cell types showing poor attachment, spreading, and growth on untreated non-tissue culture wells. In the case of SKMCs, these cells also exhibited more elongated and differentiated morphologies, potentially due to the tissue-relevant ECM composition that may expedite cell differentiation. CSdECM offers flexibility in its usage, either as a scaffold or solution. Moreover, CSdECM allows for precise control over the ECM composition and may contain more mature ECM molecules, given that it can be remodeled by cells under physiologically relevant conditions. The ECM derived from each cell sheet, formed in 3×3 cm culture devices, was used to create a 500 μL solution of CSdECM. With a high dilution factor (50× used here), it can cover much larger surface areas, thereby significantly reducing the cost of in vitro models requiring such treatments. This makes it a more affordable alternative to other products such as animal-derived collagen and Matrigel, and even recombinant vitronectin, fibronectin, and laminin.

The individual models developed in this study, based on the cell sheet engineering concept, not only offer the potential to be more accurate compared to other models, but they can also be fluidically interconnected to gain a more systemic understanding of the human body as a whole (FIGS. 22, 24 and 25). Add-ons were designed and assembled on culture devices to form chambers around each of the tissues that could be connected to each other, thereby extending the capabilities of these cell sheet-based tissue constructs.

The platform's performance and widespread adoption can be further enhanced by incorporating new features, such as sensing and tracking components, and mechanisms for creating a dynamic microenvironment. For instance, a vision system for both bright field and fluorescent microscopy could track the forces exerted by tissue constructs on the flexible pillars, effectively measuring tissue-generated forces. Additionally, the pillars could be designed to deliver electrical stimulation to cells, while the flexible culture device could be used to mechanically stimulate the constructs. The system could also facilitate the creation of constructs with different cell types, via the patterning of cells in predefined configurations on each cell sheet.

### Example 8: Magnetic Pillar for Mechanical Stimulation and/or Mechanical Testing

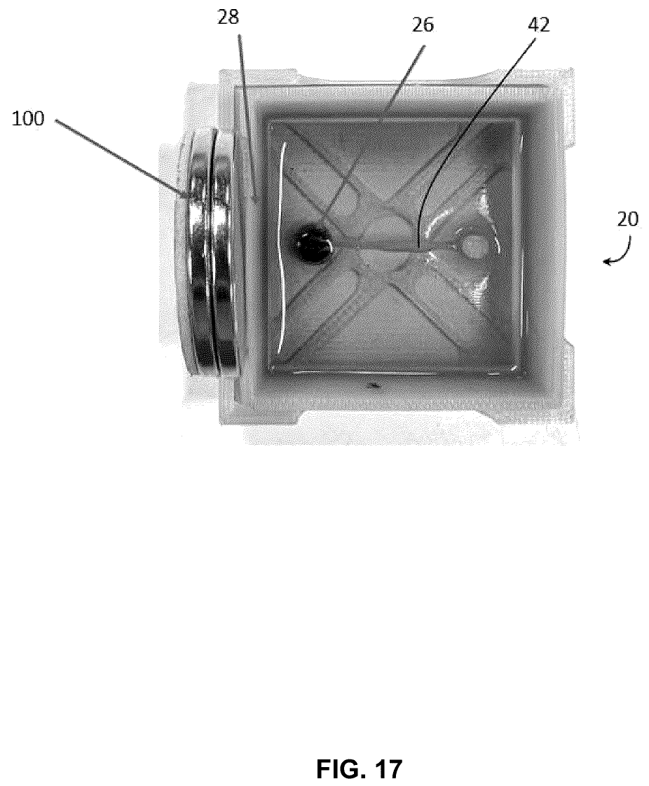

In an exemplary device 20 shown in FIG. 17, iron oxide magnetite (Fe3O4) microparticles that are 30 μm in size were used with a ratio of 250 μg of particles per mL of Ecoflex 00-30 resin. After the cell construct 42 was formed, a magnet 100 was assembled with the device 20 and the pillar 26 was deflected towards the magnet 100. By way of calculations or experiments with fibers of known diameter and modulus of elasticity, a relationship between pillar deflection 26 and tensile force applied to the pillar 26 can be determined. The deflection of the pillar 26 in response to the magnet 100 can be used to estimate the mechanical properties of the cell construct 42. The magnet 100 can also apply static mechanical stimulation to the cell construct 42.

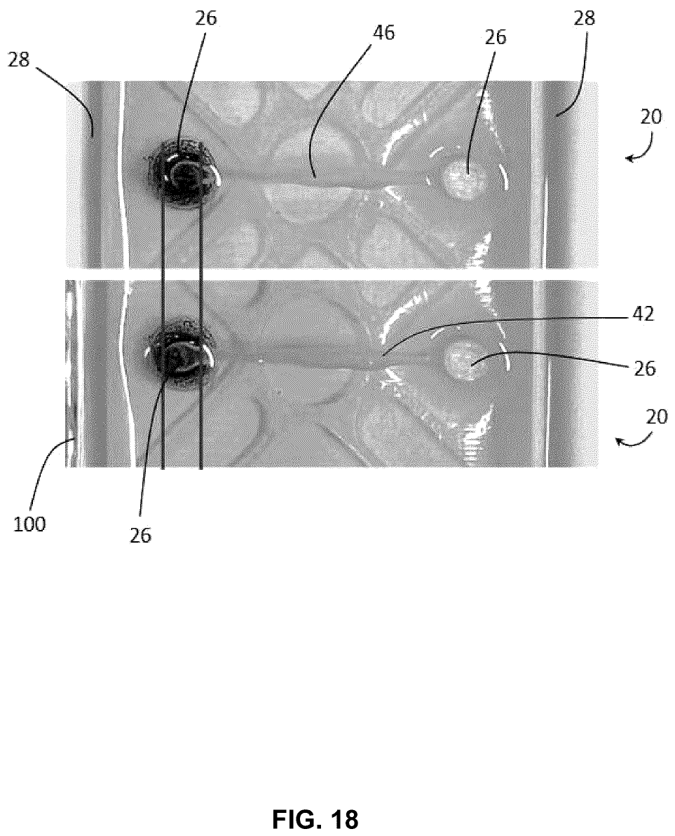

Referring to FIG. 18, the magnet 100 can be alternatively applied to the device 20 and removed. In the example shown, the magnet 100 is moved back and forth (left and right) to alternatively be separated from the wall 28 of the device or rest against the wall 28. The pillar 26 moves away from the wall 28 when the magnet 100 is removed and towards the wall 28 when the magnet 100 is replaced. The magnet 100 is used to provide dynamic or periodic mechanical stimulation to the cell construct 42.

### Example 9—Remodeling a Construct Around a Form

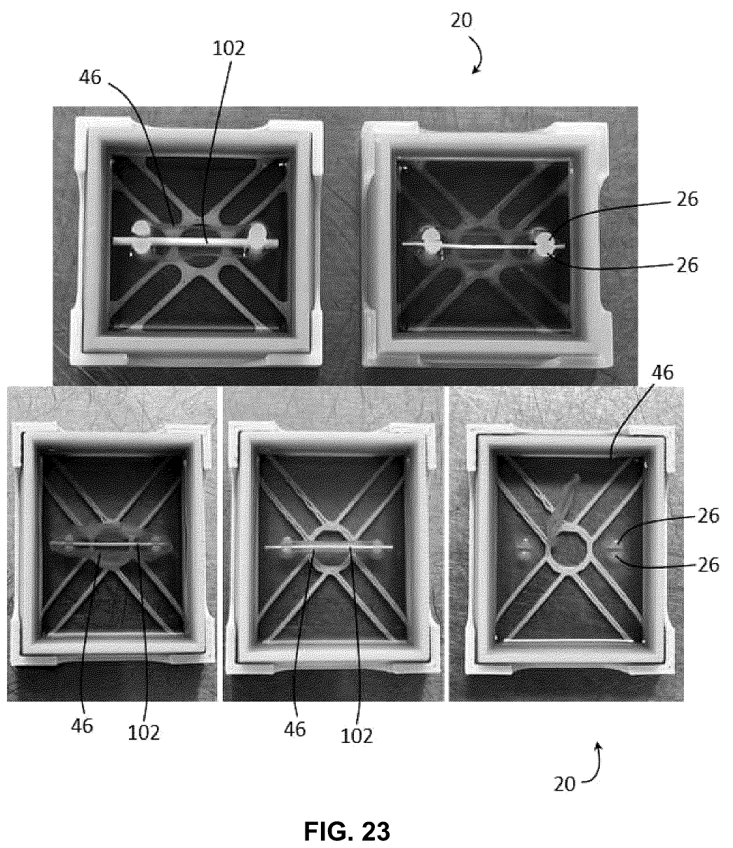

FIG. 23 shows an example in which a hollow fiber 102 is used to provide a cylindrical form. The cell culture devices 20 is modified to hold the hollow fiber 102 between two pillars 26. In the examples shown, each single pillar 26 as shown for example in FIG. 2 is replaced with two pillars 26 positioned a small distance from each other. Each end of the hollow fiber 102 rests in the spaces between two of the pillars 26. The hollow fiber 102 is thereby held above the upper surface 22 of the device 20. Alternatively, a single pillar 26 may have a notch, groove or other feature molded into the cap 34 to support an end of the hollow fiber 102.

The hollow fiber 102 can be placed on the pillars 26 before or after the cells are grown on the upper surface 22 of the membrane 24, but preferably before delamination of a cell sheet from the membrane 24. The outer diameter of the hollow fiber 102 can be selected based on the desired inner diameter of a tubular cell construct 46. Upon delamination and shrinkage of a cell sheet, a tubular cell construct 46 forms around the hollow fiber 102. After enough time is allowed for remodeling of the construct 46, the hollow fiber 102 can be removed and the tubular cell construct 46 does not collapse. Depending on factors such as the thickness and height of the pillars 26, a cell sheet can surround the hollow fiber 102 to form a full tubular construct 46 or a cell sheet can cover only the bottom of the hollow fiber 102 to form a partially tubular construct 46. Such constructs 46 can be used in applications such as blood vessel formation or as vascular grafts.

### Example 10—Anchored Cell Sheet Engineering for Drug and Cell Delivery

The technique of anchored cell sheet engineering as described herein may be expanded for use in drug, protein or cell delivery systems. In an exemplary system and method, remodeled cell sheets or fibers are produced from various cell types using an anchoring system. Once these sheets or fibers are fully formed, they undergo a decellularization process to remove cellular components while preserving the native extracellular matrix (ECM) structure. This decellularized ECM serves as a robust scaffold with several advantageous properties for drug, protein, or cell delivery applications.

### Loading and Release Mechanisms

The decellularized sheets or fibers can absorb or bind various therapeutic payloads, including small molecules, large proteins, antibodies, or cells, for example stem cells. Optionally, the decellularized sheets or fibers may be engineered or modified to modify the loading or release characteristics of the decellularized sheets or fibers in relation to a particular payload. Two exemplary techniques are optionally used for loading these payloads:

Physical Absorption: The porous and fibrous nature of the ECM allows it to act like a sponge, soaking up the payload through capillary action and physical absorption. This method is particularly useful for hydrophilic drugs or molecules that can easily penetrate the ECM structure.

Chemical Bonding: For a more targeted and stable loading, chemical modifications can be employed to facilitate the binding of the payload to the ECM. Covalent or ionic bonds may be utilized to attach specific molecules, such as growth factors or antibodies, to the decellularized sheet or fiber. In some examples, this approach not only enhances the stability of the payload but optionally allows for a more controlled release profile.

### Controlled Release and Immune Modulation

Once the payload has been absorbed or chemically bound to the anchored cell sheet or fiber, the construct can be implanted at the desired site in a patient body. The unique composition of the ECM facilitates a controlled and gradual release of the therapeutic agents. This controlled release mechanism helps in maintaining a sustained therapeutic effect, reducing the need for repeated administrations and minimizing systemic side effects.

The ECM's natural composition can also modulate the immune response in the host tissue. Upon implantation, the ECM scaffold interacts with the surrounding environment, potentially inducing an immune response that is favorable for tissue regeneration. This immune activation may help promote proper vascularization in the area, which further aids in the effective distribution of the payload. The presence of blood vessels is crucial for delivering the payload to the targeted site efficiently and can also enhance tissue integration and healing.

### Applications in Regenerative Medicine and Therapeutic Delivery

The versatility of this approach allows it to be applied in several biomedical fields, including:

Drug Delivery Systems: The anchored cell sheet-derived sheets or fibers can be used as a vehicle to deliver anti-inflammatory drugs, chemotherapeutic agents, antibiotics, or other small molecules in a localized and controlled manner. This can be especially beneficial in treating chronic wounds, cancer, or localized infections.

Large Protein and Antibody Delivery: Large proteins, growth factors, or monoclonal antibodies can be loaded onto the decellularized ECM fibers to support wound healing, promote tissue growth, or target specific pathogens. The controlled release mechanism ensures that these sensitive biomolecules remain active over an extended period.

Stem Cell Therapy: The ECM's natural properties make it an excellent carrier for stem cells or progenitor cells, providing both a structural matrix and biochemical signals that support cell attachment, survival, and differentiation. This application can be highly effective in regenerative therapies for damaged tissues or organs.

Vaccination and Immune Modulation: By loading antigens or immune-modulatory molecules onto the ECM sheets or fibers, this approach can serve as a novel vaccination strategy, either in relation to infectious disease or oncology. It could stimulate both the innate and adaptive immune responses, enhancing the body's defense mechanisms against infections or cancer.

Chronic Disease Management: Controlled release of anti-inflammatory drugs or growth factors for managing chronic diseases such as arthritis or neurodegenerative conditions.

Cancer Therapy: Targeted delivery of chemotherapeutic agents, immunotherapeutic antibodies, or small interfering RNAs (siRNAs) to solid tumors, using the ECM's ability to provide localized and sustained release.

Wound Healing: ECM fibers loaded with antibiotics or growth factors to promote healing and prevent infection in chronic wounds or diabetic ulcers.

Stem Cell Therapy: Delivery of stem cells or supporting factors such as cytokines, using the decellularized ECM as a protective environment to support stem cell survival and differentiation after implantation.

Regenerative Medicine: Using the ECM to deliver tissue-specific growth factors, promoting the regeneration of complex tissues such as cartilage, bone, or muscle. This approach can be particularly useful in applications like volumetric muscle loss (VML) or bone regeneration.

### Potential Advantages Over Conventional Delivery Methods

Protection of Payload: The ECM acts as a protective barrier that shields the therapeutic agents from rapid degradation, increasing their half-life and effectiveness at the target site. In some embodiments, adjusting the chemical structure of the ECM can also adjust the degradation of the ECM, which in turn adjusts a release pattern of the therapeutic agent.

Biocompatibility: As the ECM is derived from biological tissues, it exhibits excellent biocompatibility and reduces the risk of adverse immune reactions.

Versatility in Use: The ability to customize the loading technique and payload type makes this system adaptable for a wide range of clinical applications, from drug delivery to regenerative medicine.

### Example 11—Implant to Treat Volumetric Muscle Loss

The following example includes material derived from an article posted on the bioRxiv preprint server as Muscle-Specific ECM Fibers Made with Anchored Cell Sheet Engineering Support Tissue Regeneration in Rat Models of Volumetric Muscle Loss on Dec. 17, 2024 available at https://doi.org/10.1101/2024.12.15.628541 which is incorporated by reference herein including all of the data, Python scripts and supplementary information that was made available with it. The article describes an in vitro made, structured muscle specific ECM fiber used to treat volumetric muscle loss in a rat model through stages of integration, healing, vascularization and myogenesis.

### Abstract

Volumetric muscle loss (VML) represents a critical unmet need in regenerative medicine, with no established standard of care. This study introduces a novel therapeutic strategy using tissue-specific skeletal muscle extracellular matrix (ECM) fibers fabricated using scaffold-free anchored cell sheet engineering technology. These fibers replicate the native ECM composition and microarchitecture of skeletal muscle, incorporating essential structural and basement membrane proteins. In a rat VML model, engineered ECM fibers demonstrated a promising regenerative capacity compared to commercial porcine-derived small intestine submucosa (SIS) ECM. Over an 8-week period, the engineered fibers preserved muscle volume and weight, regulated inflammatory and fibrotic responses, and promoted vascularization. In contrast, SIS was rapidly degraded by week 4 and associated with excessive fibrotic response. Force recovery in the muscles treated with engineered ECM fibers was lower at the 8-week time point (77% compared to 91% in the control group), but histological and immunohistochemical analyses revealed newly formed, dispersed muscle fibers exclusively within the repaired muscle tissue treated with engineered ECM fibers. Importantly, only in cases where engineered ECM fibers were used, muscle weight was preserved, resulting in similar normalized force-to-weight recovery across all groups (87% in the test group vs. 88% in the control group). The histological analyses further demonstrated ongoing tissue remodeling, indicative of sustained regeneration, in contrast to the premature fibrotic healing observed in the other groups. A novel quantitative image analysis workflow using a custom Python script, enabled objective assessment of spatial tissue heterogeneity through histology and immunohistochemistry images, setting a new standard for tissue regeneration analysis. These findings establish engineered tissue-specific ECM fibers as a transformative approach for VML treatment and lay the groundwork for translation to clinical applications.

### Introduction

Skeletal muscle accounts for approximately 40% of human body mass and is characterized by its highly organized structure and remarkable regenerative capacity. This regenerative potential is primarily driven by the activation of resident muscle stem cells, known as satellite cells, and their interactions with the extracellular matrix (ECM), a complex network of proteins and proteoglycans that comprises up to 10% of muscle mass [1]. Muscle regeneration is a tightly regulated process involving intricate cellular and molecular mechanisms. Following injury, matrix metalloproteinases degrade components of the basal lamina, releasing signaling molecules that activate satellite cells. These cells are located between the basal lamina and the apical sarcolemma of myofibers, supported by key ECM components such as laminin and collagen type IV [2, 3]. Activated satellite cells contribute to ECM remodeling by upregulating fibronectin expression and secreting matrix metalloproteinases, particularly MMP-2 and MMP-9. However, the ECM itself is indispensable for facilitating the proper response of satellite cells to these signals [4]. Without intact cell-ECM interactions, even high levels of regulatory factors such as myogenin fail to promote effective muscle differentiation [5]. Moreover, in vitro studies demonstrate that satellite cells lose their mitogenic and myogenic potential when removed from their muscle-specific niche [6]. The mechanical properties of the ECM further influence satellite cell behavior and are critical for effective muscle repair, highlighting the importance of biomechanical integrity in the regenerative process [7]. While resident fibroblasts, though limited in number, are the primary contributors to ECM production and assembly, muscle cells also play an active role in ECM remodeling by secreting specific ECM components [8]. Additionally, the immune response is pivotal to muscle repair, particularly in the early stages of injury. Immune cells, such as macrophages, are recruited to the injury site to clear debris, release pro-regenerative cytokines, and support ECM remodeling, thereby facilitating successful regeneration [9, 10].

When muscle damage exceeds the tissue's regenerative capacity, this innate repair mechanism becomes overwhelmed, leading to volumetric muscle loss (VML) and resulting in permanent functional deficits. Dysregulated or chronic inflammation, often observed in severe VML, exacerbates the hindered muscle regeneration and leads to fibrosis [11, 12]. Despite advances, current clinical approaches to VML treatment face significant limitations. The gold standard, autologous muscle transfer, causes donor site morbidity and is limited to tissue availability. Similarly, cell-based therapies have struggled due to low survival and engraftment rates, limited donor cell availability, ex vivo-induced cellular changes, and the risk of teratoma formation with pluripotent stem cells. Acellular scaffolds made of synthetic biomaterials also face challenges due to insufficient cell recognition. Scaffolds composed of individual ECM components like collagen and laminin have shown some potential, but their efficacy remains limited [13-15].

ECM-based acellular products, usually from animal sources but free of antigens and major histocompatibility complexes, have emerged as promising alternatives [16]. Various tissue sources, including skeletal muscle [17, 18], dermis [19], and porcine or bovine bladder [20, 21] and small intestinal submucosa [22, 23], have been explored. However, these scaffolds often lack the ultrastructure and composition specific to muscle tissue, resulting in limited regeneration in VML cases [24, 25]. Recent innovations in more advanced ECM-based therapies for VML aim to address these shortcomings. Hybrid scaffolds that combine synthetic and natural materials are being developed to enhance both mechanical properties and bioactivity [26, 27]. Additionally, techniques such as 3D printing have shown promise in replicating the anisotropic architecture of muscle tissue, which is critical for functional regeneration [28, 29]. The effectiveness of these approaches needs to be seen but overall, acellular therapeutic strategies that promote endogenous cell recruitment represent a promising path forward, offering greater potential for clinical translation. These strategies must replicate the native muscle ECM environment, considering both compositional and structural elements to support tissue regeneration and restore functional recovery in VML [30].

In this study, an Anchored Cell Sheet Engineering platform (Shahin-Shamsabadi, A. and J. Cappuccitti, Anchored Cell Sheet Engineering: A Novel Scaffold-Free Platform for in vitro Modeling. Advanced Functional Materials, 2024. 34(13): p. 2308552, incorporated by reference herein) is utilized to generate completely scaffold- and biomaterial-free, fully formed, and functioning muscle fibers from primary myoblasts. Following decellularization, these fibers served as tissue-specific ECM scaffolds for VML treatment in rat models. Through comprehensive characterization, including proteomics, histological and immunohistochemical analysis, and scanning electron microscopy (SEM), it was demonstrated that these engineered ECM fibers possessed not only appropriate biomechanical properties but also exhibited mature, in vivo-like ECM composition and microstructure. To evaluate their regenerative potential, an 8-week study in a rat VML model was conducted, employing multiple assessment methods including histological, immunohistochemical, and functional analyses at various time points. Additionally, a novel image analysis quantification workflow was developed that provided unique insights into the presence and behavior of different cell populations, their contributions to immune and fibrotic responses, and their roles in tissue remodeling and regeneration.

### Results

Tissue-specific skeletal muscle ECM fibers with proper mechanical and microstructural properties, created using a scaffold- and biomaterial-free biofabrication platform called Anchored Cell Sheet Engineering, were used to treat VML cases in rat models. The previously published technique (Shahin-Shamsabadi, A. and J. Cappuccitti, Anchored Cell Sheet Engineering: A Novel Scaffold-Free Platform for in vitro Modeling. Advanced Functional Materials, 2024. 34(13): p. 2308552) (FIG. 1a) was used to biofabricate fully formed and functional skeletal muscle fibers. The process began by growing multiple layers of primary myoblasts on a patterned PDMS membrane and inducing their differentiation and ECM production over a 14-day period, after which cells and their ECM were easily scraped off the membrane as a coherent sheet. The anchorless sheet recognized the flexible pillars made out of Ecoflex 00-30 and utilized them as new anchors to remodel into muscle fibers. This remodeling further enhanced cellular phenotype and ECM content of the fiber, with full maturation achieved by day 18. The muscle fibers were then decellularized and treated with DNase solution to remove cell-free DNA before being used for in vivo regeneration of skeletal muscle. The proper ECM composition and microstructure of these fibers were previously demonstrated and are shown here through immunohistochemistry (IHC) and SEM characterization (FIG. 1a) as well as proteomics analysis (Supplementary Table 1 and 2, Supplementary FIGS. 1 and 2).

Proteomic analysis revealed the presence of crucial ECM as well as cell adhesion components known to be essential for skeletal muscle structure and regeneration. Notable proteins included various collagen types (types I, IV, V, and VI), which provide structural support and mechanical stability. The presence of basement membrane proteins, such as laminins and nidogen, was also confirmed. Additionally, matricellular proteins, including SPARC, thrombospondins (THBS1, THBS2), and tenascin-C, that are known to modulate cell-ECM interactions and tissue remodeling were detected. The identification of lysyl oxidases and matrix metalloproteinase-2 indicated the preservation of proteins involved in ECM crosslinking and remodeling. Proper microstructure of the fibers was assessed using SEM while proper distribution of ECM components was shown using IHC for laminin. Based on this characterization, the preservation of crucial muscle ECM components and architecture, known to be important for guiding cells in regeneration of muscle in vivo, was confirmed and the fibers were used for treating VML.

The 20% VML injuries were surgically created in the Tibialis Anterior (TA) muscle of the left leg in Sprague-Dawley rats by removing 20% of the muscle tissue by weight, 1 cm from its proximal origin. Five acellular ECM fibers (test article) were used to fill the defect site, while commercially available decellularized porcine-derived small intestine submucosa (SIS from Cook® Biotech, control article) was cut to size and used as a control. Both articles were sutured in place. In sham group, the defect was left untreated. The TA muscle epimysium and fascia were then closed. The surgical procedure is illustrated in FIG. 1b. Animals were euthanized at 2-, 4-, and 8-weeks post-surgery (group sizes detailed in Supplementary Table 3). TA muscles were harvested from both legs, with the contralateral limb serving as the native control. Samples were weighed and fixed for histological and IHC analyses.

The developments in the damaged muscle tissue were tracked across the three experimental groups using an image processing workflow that assessed the cellular and structural changes, particularly in the actively remodeling site. The workflow began with histology and IHC of tissue samples collected at different time points, followed by whole slide imaging (WSI). WSI files were processed using QuPath® software to manually detect the active site, and for each slide, a high-resolution SVG file was exported containing the contour line defining the region of interest (ROI) and grid lines (500 μm apart). A custom Python script was then developed to detect the three layers (original image, ROI contour, and grids) in each SVG file. The script masked each original image by its contour to isolate the ROI and tiled it into smaller pieces of defined sizes using the grid lines (500 μm apart) (FIG. 2a). For the image analysis purposes, only the middle part of each TA muscle containing the largest cross-section (muscle belly) was utilized. When tile-level data from all biological replicates were used, the assessment covered both inter-animal differences and tissue variability within each animal. For tile-level analysis, a mixed-effect statistical model was implemented to account for differences between tiles belonging to different animals. To analyze only the differences between animals, slide-level data were used, calculated as the mean value of tiles per slide. For slide-level analysis, a weighted t-test was used to account for the standard deviation inherent to each slide.

The Python script segmented each image based on staining-specific color definitions. Predefined color clusters were established for each staining, with individual pixels in each tile assigned to the closest matching cluster. Masson's Trichrome and Movat's Pentachrome staining were segmented into four categories (“Nuclei/Cytoplasm”, “Fibrosis”, “Muscle”; “Other” for Masson's Trichrome and “Nuclei/Elastic Fiber”, “Fibrosis”, “Muscle/Cytoplasm”, “Other” for Movat's Pentachrome), while Hematoxylin and Eosin (H&E) and IHC were divided into three segments (“Nuclei”, “Cytoplasm/Fibrosis/Muscle”, and “Other” for H&E; “Nuclei”, “Target”, and “Other” for IHC) (FIGS. 2b and c). The “Other” segment in all cases contained weakly stained regions. In H&E stained tiles, nuclei appeared dark purple, while muscle, fibrotic tissue, and cell cytoplasm showed various shades of pink and red, making this stain particularly effective for detecting nuclei. Masson's Trichrome stained nuclei and cytoplasm light red and muscle dark red, making these components difficult to differentiate reliably. However, its unique blue-to-green staining of fibrotic tissue enabled specific detection of fibrosis independent of composition. Movat's Pentachrome revealed fibrotic tissue in yellow to light brown, nuclei and elastic fibers in dark brown to black, and muscle and cytoplasm in dark red. In IHC, target antigens appeared light brown and nuclei purple, though intense target staining occasionally masked the nuclei, limiting its utility to tracking nuclei content properly. For quantification, the area coverage percentage was calculated for each segment within individual tiles. After segmentation was performed, random tiles from different staining types were visually assessed to validate the automated segmentation approach. It should be noted that in the current study only the actively remodeling part of the treatment site was considered for quantification as it could be clearly identified with high confidence. Adjacent to this area was the “transition zone” (Supplementary FIG. 3) with a combination of native muscle tissue, highly fibrotic tissue as well as newly formed myofibers that couldn't be clearly assigned to intact muscle or treatment area.

The initial assessment of muscle regeneration focused on quantifying the actively remodeling surface area through WSI histology images, measuring total muscle weights across the three time points, and evaluating electrical activity and force generation capacity at the 8-week point in both injured and contralateral muscles. Peripheral tissues including fascia, epimysium, and perimuscular adipose tissue were excluded from the analysis during area analysis, unless these components were integrated into the actively remodeling site. The surface area of the active site was measured using grid lines as reference points by the Python script (FIG. 3 a and b). The active surface area in the test group demonstrated gradual reduction over time, while the control group exhibited accelerated shrinkage with minimal active site by week 8. The sham group showed an even more rapid resolution, with no detectable active site by week 4. These observations were corroborated by muscle weight measurements at week 8, where both sham and control groups displayed significantly lower muscle weights compared to contralateral muscle, while no significant difference was observed between test and native groups (FIG. 3c). These findings indicated that regeneration had ceased in both sham and control groups by week 8, with no further improvements in muscle regeneration, suggesting that the SIS product had failed to promote VML healing in rats.

Functional assessment was performed through in vivo measurement of dorsiflexor muscle group contractions induced by percutaneous electrical stimulation of the peroneal nerve. Optimal isometric twitch torque was determined, and peak isometric force was measured in anesthetized rats at the 8-week time point. While sham and control groups demonstrated 91-92% force recovery compared to contralateral legs, the test group showed approximately 77% recovery (FIG. 3d). The full force measurements at different frequencies are included as Supplementary FIG. 4. The lower force generation in the test group was potentially attributed to ongoing active regeneration at the implantation site, suggesting either incomplete force generation capacity or impaired force transmission through the actively remodeling tissue. To determine whether the continued remodeling indicated progressive and slower regeneration or either delayed complete resolution of the test product with no regeneration, or even chronic inflammation, detailed analyses of the cellular response were conducted, as described in subsequent sections.

The nature of cell types populating the acellular ECM fibers in the test group, SIS control, and untreated injury site of the sham, as well as the extent of cellular responses were investigated using histological analysis and IHC for various targets (FIG. 4), and the quantification workflow, explained in the previous section, was applied to all samples. Histological analysis included H&E, Masson's Trichrome, and Movat's Pentachrome (FIG. 5). In all cases, native muscle tissue from the contralateral leg served as the reference point. These values represented the area coverage of each segment in the WSI images/tiles, excluding the white background. In order to evaluate differences between different parts of each large defect size, tiling was performed to break down each sample to smaller units for analysis. For the statistical analysis, a mixed-effect model was used to account for the presence of multiple tiles from the same animal, as well as for the multiple animals as separate biological replicates.

Nuclei content analysis using the nuclei segment from H&E staining, which quantified the total number of cells within the active injury site, revealed significantly higher cell counts in both the control and test groups compared to native tissue. In contrast, the cell count in the sham group was much lower, remaining comparable to the native group at week 2. This observation indicated that both the test and control articles were successfully populated by host cells; however, nuclei-free regions deeper within the test group (FIG. 4, indicated by a red star) were identified and further supported by the longer whiskers observed in the H&E nuclei analysis (Figure Sa). At week 4, both the test and control groups showed a slight increase in cell numbers, but the test group exhibited a significant decrease by week 8.

Differences between the test and control groups, when compared to both native and sham groups in the Nuclei and Elastic Fiber segment of Movat's Pentachrome (FIG. 5b), were more pronounced than those observed in the Nuclei segment of H&E, suggesting a higher elastic fiber content. This finding may indicate greater ECM deposition as early as week 2, though the implanted articles might have contributed to this elastic fiber content. By week 4, these values had decreased, and by week 8, the test group resembled native tissue. The fibrosis segment of Masson's Trichrome, which included elastic fibers, revealed significantly higher fibrotic tissue deposition in all groups compared to native tissue (Figure Sa), with the sham group showing the highest values. In the test group, these fibrosis levels remained consistent across all three time points.

Further analysis of the muscle regeneration process was performed through IHC evaluations of all groups, at both tile- and slide-levels (FIG. 6). Immune response and presence of fibrotic cells were assessed using CD68 and FSP1 targets, identifying macrophages and fibroblasts respectively. Vascularization, important for tissue regeneration, was assessed using CD31, while the onset of muscle regeneration was assessed using Desmin as an early marker of muscle differentiation. Laminin content, an important skeletal muscle ECM component involved in guiding skeletal muscle regeneration, and collagen I, both an important component of skeletal muscle integrity and a major indicator of fibrosis, were also assessed.

When considering tile-level data (FIG. 6a), all three groups (test, control, and sham) exhibited elevated levels of CD31-positive cells compared to native muscle. In the control group, these levels remained constant at week 4, whereas the test group showed a spike at week 4, followed by a decrease at week 8, though still remaining higher than native tissue. The sham group displayed a higher presence of immune cells (CD68-positive cells) compared to native muscle; however, its levels were significantly lower than those observed in the test and control groups. In the test and control groups, CD68-positive cell levels remained stable, but the test group showed a decrease by week 8. Fibrotic cell levels (FSP1-positive cells) were significantly elevated in all three groups relative to native tissue, but in the test group, these levels gradually decreased over time, reaching lower levels by week 8. While Desmin levels in the control group remained similar to those of native tissue, suggesting appropriate muscle progenitor recruitment, proper muscle fiber formation was not observed. In contrast, the test group exhibited lower Desmin levels, yet multiple newly formed, isolated, and scattered muscle fibers were detected (FIG. 7). At week 4, the control group displayed a distinct boundary between the implanted SIS and native muscle tissue, whereas the test group showed a transition zone between the native muscle and the ECM fiber implant, characterized by both newly formed myofibers and actively remodeling fibrotic tissue.

All three groups exhibited significantly higher collagen I content compared to the native tissue. While these levels remained steady between weeks 2 and 4, the test group showed a slight decline by week 8. Given the fibrotic tissue content identified in Masson's Trichrome staining, this collagen likely originated from both the onset of fibrosis and the composition of the original implanted scaffolds. Additionally, all three groups displayed elevated levels of laminin, a key glycoprotein of the ECM basement membrane. These abnormally high laminin levels may also indicate increased fibrosis. While laminin levels remained high in the control group at week 4, they progressively decreased in the test group from week 2 to week 8, approaching native tissue levels. This decline suggested effective remodeling toward a less fibrotic state in the test group.

Overall, the results indicated that the initial response in all three groups was predominantly fibrotic and inflammatory. In the sham group, fewer cells were present at week 2, as expected due to the absence of any 3D scaffolding support. The control group exhibited sustained high levels of immune activity, fibrosis, and ECM content across the first two time points; however, the lack of both new myofiber formation and recovery of tissue weight suggested that this heightened cellular activity was detrimental and counterproductive to the muscle regeneration process. In contrast, only the test group maintained muscle tissue volume and weight while displaying more moderate cellular responses, with both immune and fibrotic activities decreasing over time. This group also demonstrated a gradual remodeling of the ECM fibers and a progressive reduction in fibrotic ECM content. Proper vascularization, essential for natural healing, was observed in the test group, along with recruitment of Desmin-positive cells at levels comparable to native tissue. However, the formation of only a few scattered myofibers suggested a potential delay in the regeneration and maturation of fully developed muscle tissue, possibly requiring immune and fibrotic responses to subside further. These findings underscore the importance of longer-term studies to capture the full regenerative potential of the engineered ECM fibers, supported by the preservation of muscle volume and weight in the test group.

A unitless index, termed the Target Prevalence Index (TPI), was defined for IHC targets to complement the area percentage comparisons presented earlier (FIG. 6b). This index was calculated using slide-level data (mean values across all tiles for each animal) rather than tile-level data to facilitate a clearer comparison of overall changes across different conditions over time. To normalize target expression relative to total cell count, linear regression was employed, with the percentage of nuclei area (from H&E staining) as the independent variable and the percentage of target-positive area (from IHC staining) as the dependent variable. This normalization method was chosen over simpler approaches, such as directly dividing values, to avoid compounding uncertainties (standard deviations) from the two measurements, which could artificially inflate the variability of the final index values. The final TPI values, derived from regression residuals centered around the original target expression means, reflected the deviation of actual target expression from expected values based on cell density. This allowed for an accurate quantification of target prevalence while accounting for variations in cell density. The approach effectively normalized IHC data for both cellular (CD31, CD68, FSP1, and Desmin) and extracellular (Laminin and Collagen) targets.

The slide-level TPI enabled normalization of each target's expression relative to total cell count, facilitating more meaningful comparisons across conditions. While the TPI values exhibited trends similar to those observed in direct area comparisons, the distinctions between conditions over time became more pronounced. In the test group, TPI analysis revealed a sharp decline in CD68- and FSP1-positive cells, as well as Laminin deposition, though all three remained elevated compared to native muscle, reflecting an active but rapidly subsiding fibrotic and inflammatory response. Desmin TPI in the test group was initially lower than in native tissue, experiencing a slight decrease at week 4 before increasing by week 8, approaching native tissue levels. Notably, the control group displayed higher Desmin TPI values than both the test group and native tissue but lacked any evidence of new myofiber formation (FIG. 7). During the first two weeks, the control group also exhibited elevated CD68 and FSP1 levels compared to the test group, accompanied by a marked increase in collagen deposition. These findings, combined with lower CD31 TPI values, suggested a heightened fibrotic and inflammatory response with low vascularization, creating a less favorable environment for muscle regeneration and healing. In the sham group, TPI indices indicated a predominantly fibrotic response rather than a significant inflammatory one, further highlighting differences in the healing dynamics among the three groups.

### Discussion

Treatment of VML among other severe damages to skeletal muscle tissue poses a significant challenge in regenerative medicine due to the complexities of skeletal muscle structure and function. With no existing standard of care for VML, no tissue engineering and regenerative medicine approach has shown proper restoration of function in injured skeletal muscles yet [29]. Here, engineered tissue-specific skeletal muscle ECM fibers, fabricated using anchored cell sheet engineering, a scaffold-free biofabrication platform capable of recreating physiologically relevant ECM composition in vitro entirely by the cells, holds considerable promise as a therapeutic strategy for VML. This innovative method addresses several limitations of conventional ECM scaffolds, offering a new approach for muscle regeneration and providing a foundation for advancing VML treatment.

One of the most common treatments used for treating VML is animal-derived decellularized ECM from non-muscle tissues such as SIS and UBM. While these materials, among other acellular scaffolds, have shown efficacy in some tissue engineering applications, their capacity to support skeletal muscle regeneration has been limited [32, 33]. These animal-derived acellular ECM products often lack the mechanical robustness in such load-bearing tissues and their composition does not match the intricate skeletal muscle tissue ECM [24, 25]. It's been shown that use of animal-derived ECM in VML injury models yields insufficient muscle fiber regeneration as these scaffolds are often remodeled rapidly at the injury site and only lead to high levels of fibrotic tissue formation, inhibiting any chances of muscle regeneration [32, 34]. In cases of more complex musculoskeletal trauma, the use of animal-derived ECMs such as SIS has been shown to impair healing of the adjacent bone as well.

It is known that tissue specific microenvironments, including proper biochemical and biophysical properties, are necessary for guiding cellular behavior to promote tissue organization. This is key for effective tissue engineering and regenerative medicine of skeletal muscle tissue [35, 36]. Therefore, it is reasonable to speculate that matching the acellular ECM composition and microstructure to that of skeletal muscle tissue is of paramount importance for guiding host cells toward its regeneration. Proteomic analysis of the engineered ECM fibers in this study revealed that it closely mimicked native skeletal muscle ECM, including key structural proteins (collagen types I, IV, V, and VI), basement membrane proteins (laminins and nidogen), and matricellular proteins (SPARC and thrombospondins) (Supplementary Table 1 and 2, Supplementary FIGS. 1 and 2). IHC for laminin showed proper distribution of this key ECM protein in guiding regeneration of muscle (FIG. 1a). This is particularly important as, during myogenesis, Laminin enhances myoblasts proliferation and migration and later their alignment before fusion [37].

SEM images also showed that proper alignment and directionality of muscle ECM as well as proper microstructure including its porosity was recreated (FIG. 1a), all known to be of utmost importance in guiding cellular behavior during muscle healing and regeneration. The fiber-like form factor of the engineered ECM fibers in this study, with their tunable lengths, provides greater flexibility to match local defect shapes. Multiple fibers of different lengths can be implanted at various locations within the muscle to better align with muscle orientation requirements. This advantage is particularly important since mechanical manipulations (such as folding and securing) of sheet-like products, including animal-derived ECMs, can disrupt their integrity and compromise their biological activity when fitted to the complex geometry of VML injuries, potentially leading to more variable functional outcomes.

The use of decellularized animal-derived ECM scaffolds for treating VML has been hindered by several other factors related to the decellularization process itself. While decellularization aims to remove cellular components, residual cell-free DNA often remains in the scaffolds, potentially triggering inflammatory responses and complicating the regenerative process. Moreover, the strong detergents required to effectively decellularize thick and dense animal tissues can leave behind residues that are toxic and induce foreign body responses. These detergent residues have been shown to increase inflammatory markers like IL-1p and impede cell infiltration in animal models. The harsh decellularization methods necessary for complete cell removal can also damage the ECM itself, altering its composition, structure, and bioactivity [38-40]. These have the potential to compromise the ECM's ability to provide appropriate biochemical and biophysical cues and can impede cells natural processes essential for muscle regeneration in VML cases. It was shown that co-delivery of decellularized UBM with autologous minced muscle even negatively affected the regenerative capacity of the autografts in a rodent model of VML [41]. Meanwhile, the in vitro engineered ECM fibers in the current study, while thinner and not as dense as animal derived tissues, were decellularized individually using less harsh detergents, showing below toxic levels of both detergents and cell-free DNA (data not shown) contributing to the less severe inflammatory and fibrotic responses observed in the rat models (FIG. 5-7).

The effectiveness of the engineered ECM fibers in treating VML was assessed through histological and IHC evaluations, comparing them against a commercial SIS product as the control and a sham group with no intervention. The quantitative analysis of these images presented unique challenges that required development of a novel workflow. Traditional approaches often lack standardization and can be subject to observer bias. The methodology presented here, combining QuPath software for ROI detection with custom Python scripts for automated segmentation based on staining-specific color definitions, offered a more objective and reproducible approach to tissue analysis. The systematic tiling of samples and careful consideration of segment categories for different stain types enabled consistent quantification and proper insight extraction across all samples. Importantly, the analysis incorporated proper statistical analysis in each case, for example a mixed-effects statistical model to account for both inter-animal variability (differences between animals) and intra-animal variability (differences between different segments of the treatment area in each animal) when tile level data was used. This hierarchical approach to data analysis provided a more accurate representation of biological variation and strengthened the statistical validity of the findings compared to traditional methods that might overlook such nested structures in the data. The combination of standardized image processing and robust statistical analysis provided a framework that could be valuable for future studies in the field, particularly when dealing with complex tissue regeneration processes that exhibit high biological variability. This workflow, optimized for analyzing the spatial heterogeneity of muscle regeneration in VML models, can also be implemented in other in vitro and in vivo applications.

The temporal analysis of cellular responses underscored the benefits of tissue-specific ECM in modulating the inflammatory and fibrotic processes critical to tissue remodeling. For image analysis, only the largest cross-sections, representing areas with the greatest mass transfer limitations, were evaluated. Both animal-derived ECM and in vitro-engineered ECM fibers demonstrated proper integration with host tissue; however, some subjects in the control group exhibited fat deposition at the interface between the muscle tissue and the implanted material (FIG. 4). Such fibro-fatty tissue replacements are commonly observed in cases of repeated acute injuries, extensive volumetric muscle loss, or chronic muscle damage resulting from genetic defects, where complete muscle repair is not achievable [9]. In the test group, integration was more robust, as evidenced by the presence of a transition zone containing both newly formed myofibers and an actively remodeling implant site. In contrast, the control group exhibited a distinct boundary separating the native muscle from the implanted material (FIG. 7).

Both the engineered ECM fibers and the SIS control elicited an initial immune response characterized by CD68-positive cells. However, the engineered ECM fibers demonstrated a controlled reduction in inflammation by week 8 (FIGS. 5 and 6). Acute immune responses triggering excessive inflammation have been shown to hinder repair and regeneration [42-44]. The high immune activity observed in the control group may explain the complete degradation of the SIS by week 4, whereas the engineered ECM fibers persisted beyond 8 weeks, supported by a more moderate immune response. The vascularization dynamics further underscored the therapeutic potential of the engineered ECM fibers. Elevated levels of CD31-positive cells (FIGS. 5 and 6), along with evidence of lumen formation (FIG. 4), suggested a well-regulated angiogenic process, in stark contrast to the erratic vascularization patterns often seen with non-specific ECM materials [36, 45]. The fibrotic response, assessed through FSP1-positive cell counts, was also better controlled in the engineered ECM group, with levels decreasing over time. While early fibrotic tissue deposition is essential for maintaining tissue integrity in large injuries, persistent fibrosis, often a drawback of non-specific ECM scaffolds, can impair regeneration. This was mitigated in the engineered ECM group, likely due to the muscle-specific ECM's tailored biomechanical and biochemical properties. These findings highlight a balanced tissue remodeling process conducive to functional regeneration [26, 46]. The introduction of the TPI index, a normalized and unitless metric, further streamlined the comparison of various IHC targets, enhancing the robustness of the analysis.

Force recovery in the engineered ECM group was slightly lower (77%) than in the control group (91%), but this discrepancy may reflect active muscle regeneration rather than premature fibrosis. This hypothesis was supported by the formation of scattered new myofibers within a transition zone, indicative of ongoing integration with native tissue (FIG. 7). Rapid force recovery observed in animal-derived acellular ECM scaffolds has been associated with non-functional fibrotic tissue rather than true muscle regeneration in both mice and humans. For example, in a rodent VML model using porcine UBM, significantly fewer myosin-positive fibers were formed compared to autografts, despite higher initial force recovery. Human clinical studies have also revealed the complexity of force recovery in VML. In a trial involving 13 patients with varying muscle injuries, implantation of porcine SIS ECM resulted in an average strength improvement of 37.3% at six months. However, the study noted that these strength gains did not directly correlate with muscle regeneration [36, 47]. It's been also shown that animal-derived ECM scaffolds can only facilitate new myofiber formation when combined with muscle stem cells [48]. In contrast, the engineered ECM group in this study preserved muscle volume and weight and showed new myofiber formation suggesting a regenerative process that balanced early remodeling with long-term functional outcomes. The regenerative capacity of the engineered ECM fibers can further be improved by addition of drug/gene-releasing mechanisms [49, 50].

To fully validate these findings, further studies are needed to optimize the clinical applicability of this approach and assess its long-term effects. While this study's eight-week observation period provided valuable insights, it also highlighted the need for extended evaluations to capture the full regenerative timeline. The delayed presence of Desmin-positive cells, forming only scattered myofibers, points to the possibility of a more gradual regenerative process. Despite these limitations, the findings establish a strong foundation for the use of engineered tissue-specific ECM fibers in VML treatment. The observed balance of immune modulation, vascularization, muscle volume preservation, and weight maintenance reflected a physiologically relevant regenerative process, distinguishing this approach from traditional non-specific ECM scaffolds. With further refinement and long-term studies, this scaffold-free biofabrication technique has the potential to revolutionize VML treatment and improve outcomes for patients facing this challenging condition.

### Methods

### Biofabrication of Skeletal Muscle-Specific ECM Fibers

Acellular skeletal muscle-specific ECM fibers were fabricated using a previously published technique called anchored cell sheet engineering (Shahin-Shamsabadi, A. and J. Cappuccitti, Anchored Cell Sheet Engineering: A Novel Scaffold-Free Platform for in vitro Modeling. Advanced Functional Materials, 2024. 34(13): p. 2308552). Briefly, custom culture devices were fabricated using multiple resin types, with polydimethylsiloxane (PDMS) (Dow, SYLGARD™ 184) serving as the base membrane for two-dimensional (2D) cell culture, Ecoflex™ 00-30 (SMOOTH-ON) used for the pillars, and Ecoflex™ 00-50 (SMOOTH-ON) used for device walls. Master molds for these devices were made using fused deposition modeling (FDM) 3D printing with acrylonitrile butadiene styrene (ABS) filament. The surface patterns from the 3D-printed molds were replicated onto the PDMS membrane, facilitating cell alignment and enhancing ECM production. The PDMS membranes were treated with tannic acid to improve cell attachment. Primary rabbit skeletal muscle cells (Sigma Aldrich, RB150-05) were cultured on the PDMS culture devices over 18 days, during which cells were added four times (on days 1, 4, 7, and 10) to create a multi-layer cell culture system. The growth medium (Sigma Aldrich, RB151-500) was replaced with differentiation medium (Sigma Aldrich, 151 D-250) on day 3, which was refreshed every other day. The differentiation medium was supplemented with 100 μg/mL 2-Phospho-L-ascorbic acid trisodium (Sigma Aldrich, 49752) to stimulate ECM production. On day 14, the multi-layered cell constructs were gently scraped from the edges of the culture device using a sterile cell scraper, facilitating the formation of cell sheets. These sheets were then carefully detached from the membrane and anchored between the two pillars, transitioning to a three-dimensional (3D) culture system. Over the next 4 days, the anchored cell sheets underwent remodeling, resulting in the formation of mature, aligned muscle fibers, rich in mature muscle-specific ECM.

The mature fibers were subsequently decellularized using a detergent solution containing 0.2% v/v Triton X-100 (Sigma Aldrich, 93443) in Hank's Balanced Salt Solution (HBSS) (Sigma Aldrich, H9269). The decellularization process was carried out over 4 days at 4° C., with the detergent solution being refreshed once at the midpoint of the process. Following decellularization, the acellular ECM fibers were thoroughly washed multiple times with phosphate-buffered saline (PBS) (Sigma Aldrich, P4474) to remove any detergent residues. The ECM fibers were then treated with a 1:1000 dilution DNase solution (Thermo Scientific™, FEREN0521) at room temperature for 1 hour to eliminate any remaining DNA. After DNase treatment, several more additional washing steps with PBS was performed. All procedures were performed inside a biosafety cabinet to maintain sterility. Finally, the decellularized ECM fibers were submerged in PBS containing 1% penicillin-streptomycin, frozen at −80° C., and maintained under these conditions until required for surgery.

### Animal Study Ethics Approval

Animal experiments were conducted by Labcorp (Bedford, USA), an independent contract research organization. Following initial safety assessments, the full study protocol was reviewed and approved by the Institutional Animal Care and Use Committee (IACUC) (protocol number: 2024-NR-09). All animal procedures were performed in accordance with the NIH Guide for the Care and Use of Laboratory Animals. Animals were housed in a temperature-controlled environment (22±2° C.) with a 12-hour light/dark cycle and provided ad libitum access to standard rat chow and water. All efforts were made to minimize animal suffering and reduce the number of animals used.

### Surgical Model and Experimental Design

Sprague-Dawley rats received appropriate doses of analgesia before anesthesia. Each rat's left pelvic limb underwent aseptic preparation and draping. A lateral incision was created from the knee to ankle level, and the tibialis anterior (TA) muscle was exposed through blunt dissection. The TA fascia was incised and carefully separated from the muscle belly. Using a scalpel blade, the central 10-15 mm portion of the TA muscle was marked at its proximal and distal boundaries. Approximately 20% of the muscle mass was removed by sharp dissection, performed by holding the proximal end of the incised tissue and reflecting it distally while cutting the base with a scalpel. The excised muscle tissue was weighed to confirm the percentage of TA muscle removed. The defect was filled with five engineered ECM fibers (test article), which were secured to the remaining TA muscle using polypropylene suture at the corners and margins of the implant. For the control group, porcine SIS ECM (Cook Myotech™, G12581) was trimmed to match the defect size and sutured following the same technique. The sham group received no treatment in the defect area. The sutures, which incorporated the muscle epimysium to ensure better retention, also served as markers for the defect-implant interface during harvest. The TA muscle fascia was sutured using 5-0 Vicryl in a simple interrupted pattern, followed by skin closure using 5-0 Prolene in the same pattern. During recovery, a compression bandage was applied to the leg for 5-10 minutes. At 2-, 4-, and 8-weeks post-surgery, animals were weighed and euthanized via CO2 inhalation. TA muscles were harvested from both legs, with the contralateral leg serving as an internal control. After weighing, the dissected muscles were placed in 10% neutral buffered formalin and kept at room temperature for further analyses. (details of animals numbers per group per time point in Supplementary Table 3)

### Histological and Immunohistochemical Analyses

The fixed tissue samples underwent dehydration through a series of graded ethanol solutions, followed by xylene clearing and paraffin embedding. Using a rotary microtome, 5 μm thick sections were prepared and mounted onto positively charged glass slides. The samples were processed following standard protocols for Hematoxylin and Eosin (H&E), Masson's trichrome, and Movat's Pentachrome staining. For immunohistochemistry (IHC) procedures against CD31, CD68, FSP1, Desmin, Laminin, and Collagen type I, the sections were first deparaffinized using xylene and rehydrated through decreasing concentrations of ethanol. Antigen retrieval was conducted using citrate buffer (pH 6.0) in a pressure cooker for 20 minutes, followed by a 10-minute treatment with 0.3% hydrogen peroxide to neutralize endogenous peroxidase activity. The sections underwent blocking with 10% donkey serum in PBS for 1 hour at room temperature. Primary antibody application was performed overnight in a humidified chamber at 4° C. Following PBS washing, the sections were treated with peroxidase conjugated donkey anti mouse secondary antibody (Jackson ImmunoResearch, 715-035-150) or peroxidase conjugated donkey anti rabbit secondary antibody (Jackson ImmunoResearch, 711-035-152). The complete antibody list is provided in Supplementary Table 4. DAB chromogen (Vector Laboratories) was used to visualize the immunoreaction. After hematoxylin counterstaining, the sections were dehydrated, cleared, and sealed with permanent mounting medium. All stained slides were digitally scanned at 40× magnification using a Leica Aperio AT2 scanner (Leica Biosystems, Buffalo Grove, IL). Image analysis and quantification were performed using Aperio ImageScope software (version 12.4, Leica Biosystems).

### Image Analysis and Quantification

Whole slide images (WSIs) of stained tissue sections were analyzed using a systematic, multi-step computational approach. Initially, regions of interest (ROIs) comprising only the actively remodeling injury/treatment sites were identified in QuPath software using semi-automated boundary detection based on tissue morphology and architecture and were defined using a contour line. Reference grid lines were overlaid at 500 μm intervals for standardization of spatial measurements across samples. These three layers were then saved in SVG format to preserve imaging details. A custom image processing pipeline was developed and implemented in Python to analyze these WSIs. The code first detected the three layers from each SVG file and saved them individually as high-resolution (300 dpi) PNG files. It then isolated each ROI from the entire slide using binary masks created from the contour line, excluding surrounding regions. The masked regions were then subdivided into analysis tiles using the reference grid lines detected through adaptive thresholding and Hough transform techniques. Each tile maintained experimental traceability through a comprehensive naming convention incorporating treatment condition (Test, Sham, or Control), time point (Week 2, 4, or 8), staining type, and animal number. Empty tiles were excluded from analysis. In the next phase of the process, tissue components defined by the staining type were quantified using a color-based segmentation approach specific to each staining type. For histological stains, H&E (segments: “Nuclei” dark purple, “Cytoplasm/Fibrosis/Muscle” pink/red, “Other” weakly stained), Masson's Trichrome (segments: “Nuclei/Cytoplasm” light red, “Fibrosis” blue/green, “Muscle” dark red, “Other” weakly stained), and Movat's Pentachrome (segments: “Nuclei/Elastic Fiber” dark brown/black, “Fibrosis” yellow/light brown, “Muscle/Cytoplasm” dark red, “Other” weakly stained) staining, tissue components were categorized based on their characteristic colors. For IHC targets (segments: “Nuclei” purple, “Target” brown, “Other” weakly stained), the analysis distinguished between DAB-positive regions (brown) and negative regions (purple counterstain). Segmentation was performed using a nearest-neighbor classification in RGB color space, with predefined color clusters established through comprehensive analysis of representative images. The area coverage for the stained part of each tile was measured, and the values were recorded for each condition at each time point. The distribution of tissue components across conditions and time points was visualized using box plots with median (horizontal line), interquartile range (box), and min/max values (whiskers) for each anatomical location. For statistical analysis, a mixed-effects model was employed to account for both biological replicates (different animals) and technical replicates (multiple tiles per slide from each animal). This was then followed by a pairwise t-tests with unequal variance assumption (Welch's t-test) between groups at different timepoint. P-values were adjusted for multiple comparisons using the Bonferroni correction method and values less than 0.05 were considered statistically significant.

To provide a more meaningful temporal comparison between different conditions an index called Target Prevalence Index (TPI) was defined for IHC targets by fitting a linear regression between the percentage of nuclei area from H&E staining as the independent variable and the percentage of IHC target-positive area as the dependent variable. The TPI values, derived from regression residuals normalized to the original target expression means, provided a unitless measure of target abundance independent of local cell density. TPI values were presented using the same box plots. For calculating TPI values, slide level data (mean of all tile values for each slide) were used with a weighted two-sample t-tests with Bonferroni correction implemented for statistical analysis purposes to account for standard deviations calculated from tile level data.

### Neuromuscular Function Assessment

Muscle performance was measured in vivo at week 8 with a 305C muscle lever system (Aurora Scientific Inc., Aurora, CAN). Rats were anesthetized with isoflurane (4-5% induction, 2% maintenance) and placed on a thermostatically controlled platform at 37° C. The knee was isolated using a 25G needle inserted through the proximal tibia and secured to a stabilization device, with the foot firmly fixed to a footplate on the motor shaft. Contractions of dorsiflexor muscle group were elicited by percutaneous electrical stimulation of the peroneal nerve and optimal isometric twitch torque determined by measuring responses at 5° increments throughout ankle range of motion. A series of stimulations were then performed at increasing frequency of stimulation (10-150 Hz, 0.2 ms pulse width, 500 ms train duration) and maximal peak isometric force determined at each frequency. Maximum force values were visualized using box plots, and force-frequency relationships were plotted for each experimental group. The method follows the protocol provided in reference [51]. Statistical analysis was performed using one-way ANOVA followed by pairwise t-tests with unequal variance assumption (Welch's t-test) to compare differences between groups. P-values less than 0.05 were considered statistically significant.

### Conclusions

This study established engineered tissue-specific ECM fibers, fabricated in vitro using a completely scaffold- and biomaterial-free platform, as a promising therapeutic strategy for VML, offering significant advantages over traditional non-specific and often animal derived ECM scaffolds. The fibers demonstrated the ability to preserve muscle volume and weight while promoting controlled immune and fibrotic responses and vascularization. Unlike conventional animal-derived ECM scaffolds, which are prone to rapid degradation and excessive and chronic inflammation and fibrosis, the engineered fibers exhibited a tailored regenerative process with progressively reduced fibrotic tissue deposition and sustained tissue remodeling. The use of a gentler and more effective decellularization process preserved the biochemical and structural integrity of the ECM, minimizing inflammatory complications commonly associated with traditional animal-derived ECMs. Although the use of engineered ECM fibers led to the formation of scattered myofibers, force recovery was slightly lower than in the control group. Histological and immunohistochemical findings suggested that this discrepancy reflects active regeneration rather than impaired healing, highlighting the potential for sustained long-term functional recovery. Furthermore, the novel image analysis workflow developed in this study offered a reproducible and objective framework for assessing spatial tissue heterogeneity, advancing the field of regenerative medicine in general. While the eight-week timeline provided valuable insights, extending the observation period is critical to fully elucidate the regenerative potential of these engineered fibers. With continued refinement and longer-term studies, this scaffold-free biofabrication approach holds the potential to transform VML treatment, paving the way for improved outcomes in patients with severe muscle injuries.

### Supplementary Information

SUPPLEMENTARY TABLE 1
List of identified extracellular matrix proteins
identified in acellular fibers using proteomics analysis. The list is generated using the
STRING database.
Detected proteins

| | |
|---|---|
| SERPINF1 | Serpin family F member 1; Belongs to the serpin family. |
| | (418 aa) |
| COL6A2 | Uncharacterized protein. (425 aa) |
| LOXL1 | Lysyl oxidase like 1. (535 aa) |
| VCAN | Versican. (3473 aa) |
| CCN2 | Cellular communication network factor 2. (437 aa) |
| GLG1 | Golgi glycoprotein 1. (1225 aa) |
| THBS1 | Thrombospondin 1. (1171 aa) |
| FBN1 | Fibrillin 1. (2831 aa) |
| LUM | Lumican. (338 aa) |
| EMILIN1 | Elastin microfibril interfacer 1. (982 aa) |
| COL6A3 | Uncharacterized protein. (562 aa) |
| FBN2 | Fibrillin 2. (2912 aa) |
| ECM2 | Extracellular matrix protein 2. (691 aa) |
| TIMP3 | Metalloproteinase inhibitor 3; Complexes with |
| | metalloproteinases (such as collagenases) and irreversibly |
| | inactivates them by binding to their catalytic zinc cofactor. |
| | May form part of a tissue-specific acute response to |
| | remodeling stimuli (By similarity). (211 aa) |
| LTBP1 | Latent transforming growth factor beta binding protein 1. |
| | (1705 aa) |
| LAMA4 | Laminin subunit alpha 4. (1732 aa) |
| PRELP | Proline and arginine rich end leucine rich repeat protein. |
| | (380 aa) |
| SPARC | SPARC; Appears to regulate cell growth through interactions |
| | with the extracellular matrix and cytokines. Binds calcium |
| | and copper, several types of collagen, albumin, |
| | thrombospondin, PDGF and cell membranes. There are two |
| | calcium binding sites; an acidic domain that binds 5 to 8 |
| | Ca(2+) with a low affinity and an EF-hand loop that binds a |
| | Ca(2+) ion with a high affinity (By similarity); Belongs to the |
| | SPARC family. (315 aa) |
| COL1A2 | Collagen alpha-2(1) chain; Type I collagen is a member of |
| | group I collagen (fibrillar forming collagen); Belongs to the |
| | fibrillar collagen family. (1364 aa) |
| LAMC1 | Laminin subunit gamma 1. (998 aa) |
| COL4A1 | Collagen type IV alpha 1 chain. (1637 aa) |
| COL4A2 | Collagen type IV alpha 2 chain. (1564 aa) |
| HTRA1 | HtrA serine peptidase 1. (338 aa) |
| LAMB1 | Laminin subunit beta 1. (1736 aa) |
| COL5A2 | Collagen type V alpha 2 chain. (1502 aa) |
| MGP | Matrix Gla protein; Associates with the organic matrix of |
| | bone and cartilage. Thought to act as an inhibitor of bone |
| | formation. (103 aa) |
| HSD17B12 | Hydroxysteroid 17-beta dehydrogenase 12; Belongs to the |
| | short-chain dehydrogenases/reductases (SDR) family. (312 |
| | aa) |
| HSPG2 | Heparan sulfate proteoglycan 2. (4207 aa) |
| G1TVW1_RABIT | Fe20G dioxygenase domain-containing protein. (273 aa) |
| TNC | Tenascin C. (2384 aa) |
| PXDN | Peroxidasin. (1411 aa) |
| THBS2 | Thrombospondin 2. (1170 aa) |
| ASPN | Asporin. (373 aa) |
| BGN | Biglycan; May be involved in collagen fiber assembly. (394 |
| | aa) |
| CRISPLD2 | Cysteine rich secretory protein LCCL domain containing 2. |
| | (471 aa) |
| FBLN5 | Fibulin 5. (453 aa) |
| COL5A1 | Fibrillar collagen NC1 domain-containing protein. (383 aa) |
| MMP2 | 72 kDa type IV collagenase; Ubiquitinous metalloproteinase |
| | that is involved in diverse functions such as remodeling of |
| | the vasculature, angiogenesis, tissue repair, tumor invasion, |
| | inflammation, and atherosclerotic plaque rupture. As well as |
| | degrading extracellular matrix proteins, can also act on |
| | several nonmatrix proteins such as big endothelial 1 and |
| | beta- type CGRP promoting vasoconstriction. Also cleaves |
| | KISS at a Gly-\|-Leu bond. Appears to have a role in |
| | myocardial cell death pathways. Contributes to myocardial |
| | oxidative stress by regulating the activity of GSK3beta. |
| | Cleaves GSK3 [ ... ] (710 aa) |
| GPC6 | Glypican 6; Cell surface proteoglycan that bears heparan |
| | sulfate. Belongs to the glypican family. (555 aa) |
| ENSOCUP00000037812 | Uncharacterized protein. (1162 aa) |
| NID1 | Uncharacterized protein. (1000 aa) |
| LOX | Lysyl oxidase. (419 aa) |
| ENSOCUP00000044803 | Uncharacterized protein. (811 aa) |
| ENSOCUP00000047220 | Uncharacterized protein. (573 aa) |

Predicted functional proteins

| | |
|---|---|
| COL3A1 | Collagen type III alpha 1 chain. |
| POSTN | Periostin. |
| DCN | Decorin; May affect the rate of fibrils formation; Belongs to |
| | the small leucine-rich proteoglycan (SLRP) family. SLRP |
| | class I subfamily. |
| COL1A1 | Collagen alpha-1(I) chain. |
| ENSOCUP00000040886 | Uncharacterized protein. |
| FN1 | Fibronectin; Fibronectins bind cell surfaces and various |
| | compounds including collagen, fibrin, heparin, DNA, and |
| | actin. Fibronectins are involved in cell adhesion, cell motility, |
| | opsonization, wound healing, and maintenance of cell |
| | shape. Involved in osteoblast compaction through the |
| | fibronectin fibrillogenesis cell-mediated matrix assembly |
| | process, essential for osteoblast mineralization. Participates |
| | in the regulation of type I collagen deposition by osteoblasts |
| | (By similarity). |
| DPT | Dermatopontin. |
| COL4A3 | Collagen type IV alpha 3 chain. |
| COL12A1 | Collagen alpha-1(XII) chain; Type XII collagen interacts with |
| | type I collagen-containing fibrils, the COL1 domain could be |
| | associated with the surface of the fibrils, and the COL2 and |
| | NC3 domains may be localized in the perifibrillar matrix; |
| | Belongs to the fibril-associated collagens with interrupted |
| | helices (FACIT) family. |
| COL15A1 | Collagen type XV alpha 1 chain. |

SUPPLEMENTARY TABLE 2
List of identified cell-extracellular matrix adhesion
proteins identified in acellular fibers using proteomics analysis.
The list is generated using the STRING database.
Detected proteins

| | |
|---|---|
| EMILIN1 | Elastin microfibril interfacer 1. (982 aa) |
| LAMC1 | Laminin subunit gamma 1. (998 aa) |
| LAMB1 | Laminin subunit beta 1. (1736 aa) |
| TNC | Tenascin C. (2384 aa) |

Predicted functional proteins

| | |
|---|---|
| ITGA9 | Integrin_alpha2 domain-containing protein; Belongs to the integrin |
| | alpha chain family. |
| ITGA10 | Integrin subunit alpha 10; Belongs to the integrin alpha chain |
| | family. |
| ITGA7 | Integrin subunit alpha 7; Belongs to the integrin alpha chain family. |
| ITGA1 | Integrin subunit alpha 1; Belongs to the integrin alpha chain family. |
| ITGA11 | Integrin subunit alpha 11; Belongs to the integrin alpha chain |
| | family. |
| ITGA4 | Integrin subunit alpha 4; Belongs to the integrin alpha chain family. |
| ITGA2 | Integrin subunit alpha 2; Belongs to the integrin alpha chain family. |
| ITGB8 | Integrin beta-8; Integrin alpha-V:beta-8 (ITGAV:ITGB8) is a |
| | receptor for fibronectin (By similarity). It recognizes the sequence |
| | R-G-D in its ligands (By similarity). Integrin alpha-V:beta-6 |
| | (ITGAV:ITGB6) mediates R-G-D-dependent release of transforming |
| | growth factor beta-1 (TGF-beta- 1) from regulatory Latency- |
| | associated peptide (LAP), thereby playing a key role in TGF-beta-1 |
| | activation on the surface of activated regulatory T-cells (Tregs) (By |
| | similarity). Required during vasculogenesis (By similarity). |
| ITGAV | Integrin subunit alpha V; Belongs to the integrin alpha chain family. |
| SV2C | Synaptic vesicle glycoprotein 2C. |

SUPPLEMENTARY TABLE 3

| Number of animals for different conditions and time | | | |
|---|---|---|---|
| points. | | | |
| Time point | Test | Ctrl | Sham |
| Week 2 | 5 | 4 | 5 |
| Week 4 | 5 | 5 | 4 |
| Week 8 | 6 | 5 | 4 |

SUPPLEMENTARY TABLE 4
List of primary and secondary antibodies used for
immunohistochemistry.
Primary antibodies

| Target | Dilution | Source |
|---|---|---|
| Rabbit anti-Laminin | 1:500 | Abcam, ab11575 |
| Rabbit anti-Desmin | 1:300 | Abcam, ab32362 |
| Rabbit anti-Collagen I | 1:400 | Abcam, ab270993 |
| Rabbit anti-FSP1 | 1:200 | Sigma, ABF32 |
| Rabbit anti-CD31 | 1:200 | Abcam, ab182981 |
| Rabbit anti-CD68 | 1:100 | R&D Systems, MAB101141 |

Secondary antibodies

| Target | Source |
|---|---|
| Peroxidase conjugated donkey | Jackson ImmunoResearch, 715- |
| anti mouse antibody | 035-150 |
| Peroxidase conjugated donkey | Jackson ImmunoResearch, 715- |
| anti mouse antibody | 035-152 |

The examples provided above are intended to enhance and further enable the disclosure and not to limit the invention. Other embodiments of the invention may be made or used within the scope of the invention, which is defined by the following claims.

I/We claim:

1. A method for producing a cell construct comprising the steps of,
growing a cell sheet;
locating the cell sheet around one or more anchors; and,
culturing the cell sheet, whereby the cell sheet remodels to form a cell construct on the anchors.

2. The method of claim 1 comprising,
growing cells in one or more layers attached to a substrate; and,
releasing at least a portion of the one or more layers from the substrate, thereby forming the cell sheet.

3. The method of claim 2 wherein the anchors extend from the substrate and wherein an outer perimeter of the one or more layers attached to the substrate surrounds the plurality of anchors.

4. The method of claim 2 wherein the one or more layers of cells are grown attached to the substrate for at least one week and produce ECM while attached to the substrate.

5. The method of claim 1 wherein the substrate is hydrophilic and optionally grooved.

6. The method of claim 1 wherein the cell sheet has multiple layers.

7. The method of claim 1 performed essentially without adding exogenous ECM materials or hydrogels.

8. The method of claim 1 wherein the cell construct is a fiber.

9. The method of claim 1 wherein releasing the one or more layers comprises one or more of scraping the edges of the one or more layers away from the membrane and growing the layers to a thickness that spontaneously detaches from the substrate.

10. The method of claim 1 comprising decellularizing the construct.

11. The method of claim 10 comprising using the decellularized construct as a scaffold.

12. The method of claim 10 comprising implanting the decellularized construct, for example to treat volumetric muscle loss.

13. The method of claim 10 comprising re-cellularizing the decellularized construct.

14. The method of claim 1 wherein the cells are skeletal muscle cells of skeletal muscle progenitor cells.

15. The method of claim 1 wherein the cell construct comprises extra-cellular material (ECM) produced by the cells, the method further comprising decellularizing the cell construct to produce an ECM construct; and, loading a therapeutic agent into the ECM construct.

16. The method of claim 15 wherein the therapeutic agent comprises a drug, a protein or a cell.

17. The method of claim 15 comprising placing the ECM construct in a patient.

18. A scaffold consisting essentially of ECM produced by cells, optionally cells cultured according to claim 1, wherein the cells have been removed from the scaffold after producing the ECM.

19. The scaffold of claim 18 wherein the cells comprise skeletal muscle cells and the scaffold is in the form of a fiber.

20. The scaffold of claim 15 comprising a therapeutic agent loaded into the ECM scaffold.
